(19) United States
(12) Patent Application Publication
(10) Pub. No.: US 2021/0379066 A1
(43) Pub. Date: Dec. 9, 2021

(54) COMBINATIONS OF POSITIVE ALLOSTERIC MODULATORS AND NICOTINIC ACETYLCHOLINE RECEPTOR AGONISTS FOR TREATING OCULAR CONDITIONS

(71) Applicant: Oyster Point Pharma, Inc., Princeton, NJ (US)

(72) Inventor: Jeffrey Alan NAU, Pennington, NJ (US)

(21) Appl. No.: 17/144,548

(22) Filed: Jan. 8, 2021

Related U.S. Application Data
(63) Continuation of application No. PCT/US2019/041013, filed on Jul. 9, 2019.
(60) Provisional application No. 62/814,754, filed on Mar. 6, 2019.
(60) Provisional application No. 62/773,993, filed on Nov. 30, 2018.
(60) Provisional application No. 62/696,268, filed on Jul. 10, 2018.

(51) Int. Cl. A61K 31/506; A61K 9/00; A61K 31/565; A61K 31/7048; A61K 31/55; A61K 31/353; A61K 31/045; A61K 31/429; A61K 31/473; A61K 31/404; A61K 31/13; A61K 31/4439; A61K 31/4535; A61P 27/04

(52) U.S. Cl. CPC A61K 31/506; A61K 9/0043; A61K 31/565; A61K 31/7048; A61K 31/55; A61K 31/353; A61P 27/04; A61K 31/429; A61K 31/473; A61K 31/404; A61K 31/13; A61K 31/4439; A61K 31/4535; A61K 31/045

(57) ABSTRACT

Described herein are methods and pharmaceutical formulations for treating dry eye disease, increasing tear production, and reducing ocular discomfort.

### CROSS REFERENCE TO RELATED APPLICATIONS

This application is a continuation of International Patent Application No. PCT/US2019/041013, filed Jul. 9, 2019, which claims the benefit of U.S. Provisional Application Nos. 62/696,268, filed Jul. 10, 2018; 62/773,993, filed Nov. 30, 2018; and 62/814,754, filed Mar. 6, 2019, the contents of which are incorporated herein by reference in their entirety.

### TECHNICAL FIELD

The disclosure describes the use of a nicotinic acetylcholine receptor agonist and a positive allosteric modulator in treating dry eye disease, ocular discomfort, and increasing tear production.

### BACKGROUND

Dry eye disease is a multifactorial, age-related disorder of the ocular surface resulting in severe pain, visual impairment, tear film hyperosmolarity and instability, inflammation, and corneal wounding. Dry eye disease may be characterized by a loss of homeostasis of the tear film. While the prevalence of dry eye disease is difficult to report due to varying definitions and diagnostic criteria, it is estimated that between 5% and 35% of the world's population over 50 years old suffers from this condition. In the United States (US), it is estimated that as many as 3.2 million women and 1.7 million men over the age of 50 have dry eye disease, with a projected 40% increase in the number of patients by 2030. Risk factors for dry eye disease include increasing age, certain medications and genetic diseases, increasing number of patients undergoing refractive surgeries that disrupt the cornea, and fluctuations in sex hormones (e.g., that may occur during or after menopause).

Dry eye disease is progressive in nature, and fundamentally results from insufficient tear coverage on the surface of the eye in combination with ocular surface inflammation. This poor tear coverage prevents healthy gas exchange and nutrient transport for the ocular surface, promotes cellular desiccation, and creates a poor refractive surface for vision. Poor tear coverage typically results from insufficient aqueous tear production from the lacrimal glands and/or excessive evaporation of aqueous tears resulting from dysfunction of the Meibomian glands. Low tear volume causes a hyperosmolar environment that induces an inflamed state of the ocular surface. This inflammatory response induces apoptosis of the surface cells which, in turn, prevents proper distribution of the tear film on the ocular surface. This initiates a cycle where more inflammation can ensue, causing more surface cell damage. Additionally, the neural control loop which controls reflex tear activation is disrupted because the sensory neurons on the surface of the eye are damaged. As a result, fewer tears are secreted and a second cycle develops which results in further progression of the disease. Patients with severe cases of dry eye disease are at risk for serious ocular health deficiencies such as corneal ulceration, and can experience a quality of life deficiency comparable to that of moderate-severe angina.

Treatment options for dry eye disease include artificial tear substitutes, ointments, gels, warm compresses, environmental modification, topical cyclosporine (Restasis®), lifitegrast (Xiidra®), and omega-3 fatty acid supplements. Unfortunately, none of these options provides substantial efficacy for a majority of the population and these options have poor patient compliance due to side effects, including stinging/burning and bad taste after instillation (dysgeusia).

### SUMMARY

Provided herein is a method of treating dry eye disease, increasing tear production, or reducing ocular discomfort in an individual in need thereof, comprising administering a first dose, and optionally one or more subsequent doses, of an effective amount of a nicotinic acetylcholine receptor (nAChR) agonist, or a pharmaceutically acceptable salt thereof, and a first dose, and optionally one or more subsequent doses, of an effective amount of a positive allosteric modulator (PAM), or a pharmaceutically acceptable salt thereof, into a nasal cavity of the individual in need thereof, wherein the nAChR agonist, or a pharmaceutically acceptable salt thereof, is varenicline, or a pharmaceutically acceptable salt thereof, or compound 1 having the structure

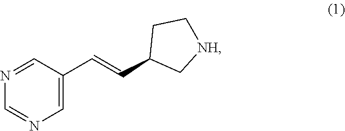 [SMILES: C(=C\[C@H]1CCNC1)/c1cncnc1]

or a pharmaceutically acceptable salt thereof, wherein the method results in the effective treatment of the individual in need thereof.

Provided herein is a compound for use in a method of treating dry eye disease, increasing tear production, or reducing ocular discomfort in an individual in need thereof, wherein the compound is a nAChR agonist, or a pharmaceutically acceptable salt thereof, wherein the method comprises administering a first dose, and optionally one or more subsequent doses, of an effective amount of a nAChR agonist, or a pharmaceutically acceptable salt thereof, a first dose, and optionally one or more subsequent doses, of an effective amount of a PAM, or a pharmaceutically acceptable salt thereof, into a nasal cavity of the individual in need thereof, wherein the nAChR agonist, or a pharmaceutically acceptable salt thereof, is varenicline, or a pharmaceutically acceptable salt thereof, or compound 1, or a pharmaceutically acceptable salt thereof. In some of the embodiments described herein, the method results in the effective treatment of the individual in need thereof.

Provided herein is a compound for use in a method of treating dry eye disease, increasing tear production, or reducing ocular discomfort in an individual in need thereof, wherein the compound is a PAM, or a pharmaceutically acceptable salt thereof, wherein the method comprises administering a first dose, and optionally one or more subsequent doses, of an effective amount of a nAChR agonist, or a pharmaceutically acceptable salt thereof, and a first dose, and optionally one or more subsequent doses, of an effective amount of a PAM, or a pharmaceutically acceptable salt thereof, into a nasal cavity of the individual in need thereof, and wherein the nAChR agonist, or a pharmaceutically acceptable salt thereof, is varenicline, or a pharmaceutically acceptable salt thereof, or compound 1, or a pharmaceutically acceptable salt thereof. In some of the embodiments described herein, the method results in the effective treatment of the individual in need thereof.

Provided herein is a combined preparation of: (i) a nAChR agonist, or a pharmaceutically acceptable salt thereof, and (ii) a PAM, or a pharmaceutically acceptable salt thereof, for simultaneous, separate or sequential use in a method of treating dry eye disease, increasing tear production, or reducing ocular discomfort in an individual in need thereof, wherein the nAChR agonist is varenicline, compound 1, or a pharmaceutically acceptable salt of either of the foregoing, and wherein the method comprises administering a first dose, and optionally one or more subsequent doses, of an effective amount of the nAChR agonist, or pharmaceutically acceptable salt thereof, and a first dose, and optionally one or more subsequent doses, of an effective amount of the PAM, or pharmaceutically acceptable salt thereof, into a nasal cavity of the individual in need thereof. In some of the embodiments described herein, the method results in the effective treatment of the individual in need thereof.

Provided herein is a pharmaceutical formulation for local administration into the nasal cavity of an individual, wherein the pharmaceutical formulation is for use in a method of treating dry eye disease, increasing tear production, or reducing ocular discomfort in an individual in need thereof, wherein the pharmaceutical formulation comprises a nAChR agonist, or a pharmaceutically acceptable salt thereof, and a PAM, or a pharmaceutically acceptable salt thereof, and wherein the nAChR agonist, or a pharmaceutically acceptable salt thereof, is varenicline, or a pharmaceutically acceptable salt thereof, or compound 1, or a pharmaceutically acceptable salt thereof. In some embodiments disclosed herein, the nAChR agonist, or a pharmaceutically acceptable salt thereof, is formulated for nasal administration.

Provided herein is the use of a nAChR agonist, or a pharmaceutically acceptable salt thereof, in the manufacture of a medicament, wherein the medicament is used in a method of treating dry eye disease, increasing tear production, or reducing ocular discomfort, wherein the method comprises administering a first dose, and optionally one or more subsequent doses, of an effective amount of a nAChR agonist, or a pharmaceutically acceptable salt thereof, and a first dose, and optionally one or more subsequent doses, of an effective amount of a PAM, or a pharmaceutically acceptable salt thereof, into a nasal cavity of the individual in need thereof, and wherein the nAChR agonist, or a pharmaceutically acceptable salt thereof, is varenicline, or a pharmaceutically acceptable salt thereof, or compound 1, or a pharmaceutically acceptable salt thereof.

Provided herein is the use of a PAM, or a pharmaceutically acceptable salt thereof, in the manufacture of a medicament, wherein the medicament is used in a method of treating dry eye disease, increasing tear production, or reducing ocular discomfort, wherein the method comprises administering a first dose, and optionally one or more subsequent doses, of an effective amount of a nAChR agonist, or a pharmaceutically acceptable salt thereof, and a first dose, and optionally one or more subsequent doses, of an effective amount of a PAM, or a pharmaceutically acceptable salt thereof, into a nasal cavity of the individual in need thereof, and wherein the nAChR agonist, or a pharmaceutically acceptable salt thereof, is varenicline, or a pharmaceutically acceptable salt thereof, or compound 1, or a pharmaceutically acceptable salt thereof.

Provided herein is a kit comprising a nAChR agonist, or a pharmaceutically acceptable salt thereof, and a PAM, or a pharmaceutically acceptable salt thereof, wherein the kit is used in a method of treating dry eye disease, increasing tear production, or reducing ocular discomfort, wherein the method comprises administering a first dose, and optionally one or more subsequent doses, of an effective amount of a nAChR agonist, or a pharmaceutically acceptable salt thereof, and a first dose, and optionally one or more subsequent doses, of an effective amount of a PAM, or a pharmaceutically acceptable salt thereof, into a nasal cavity of the individual in need thereof, and wherein the nAChR agonist, or a pharmaceutically acceptable salt thereof, is varenicline, or a pharmaceutically acceptable salt thereof, or compound 1, or a pharmaceutically acceptable salt thereof.

### BRIEF DESCRIPTION OF THE DRAWINGS

FIG. 1 shows an example of the visual analog scale used in the Eye Dryness test for individuals to record the severity of their symptoms.

FIG. 2 shows a grading diagram of the division of the corneal surface based on the NEI/Industry Workshop scale.

FIG. 3 shows an example of a questionnaire used in determining the OSDI score of an individual.

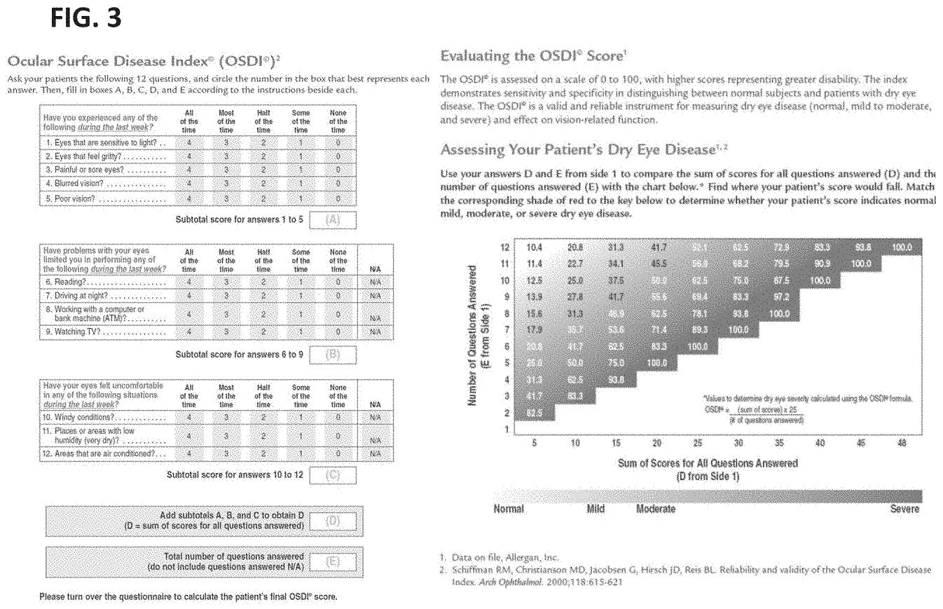

### DETAILED DESCRIPTION

The present disclosure provides for methods of improving or re-establishing tear film homeostasis. In some embodiments, the individual in need thereof lacks tear film homeostasis.

The present disclosure provides for methods of treating dry eye disease, increasing tear production, and reducing ocular discomfort, comprising administering to an individual in need thereof a positive allosteric modulator (PAM), or a pharmaceutically acceptable salt thereof, and a nAChR agonist, or a pharmaceutically acceptable salt thereof. The present disclosure also provides methods of treating dry eye disease, increasing tear production, and reducing ocular discomfort, comprising administering to an individual in need thereof a PAM, or a pharmaceutically acceptable salt thereof. In some embodiments, the ocular discomfort is associated with contact lens intolerance. In some embodiments, the individual in need thereof has discomfort wearing contact lenses.

A brief introduction and discussion of PAMs follows.

In biochemistry and pharmacology, an allosteric modulator (allo- from the Greek meaning “other”) is a substance which indirectly influences (modulates) the effects of a primary ligand that directly activates or deactivates the function of a target protein. Allosteric modulators bind to a site distinct from that of the orthosteric binding site. They stabilize a conformation of the protein structure that affects either the binding or the efficacy of the primary ligand. Pure modulators have no direct effect on the function of the protein target. The modulatory properties are interdependent with the ternary complex consisting of the target protein, the primary ligand and the modulator.

nAChRs are a class of pentameric ligand-gated ion channels that have high affinity and selectivity for both nicotine and acetylcholine (which resembles nicotine in its protonated form) and comprise combinations of alpha and beta subunits. Examples of nAChR subtypes include, but are not limited to, alpha3beta4, alpha4beta2, alpha3alpha5beta4, and alpha4alpha6beta2. An important nAChR receptor subtype involved in instigating the nasolacrimal reflex, for example, is the alpha4beta2 subtype located on the trigeminal nerve endings in the nasal mucosa.

Reference to an “alpha7” nAChR herein, refers to the homomeric alpha7 subtype, wherein the pentameric subunits of nAChR are composed entirely of alpha7 subunits. Thus, a nAChR agonist that binds and activates nAChR alpha7 is an agonist that binds and activates nAChR homomeric alpha7 receptor. In some embodiments described herein, the nAChR agonist is an alpha7 receptor agonist. In some embodiments described herein, the nAChR agonist is not an alpha7 receptor agonist. In some embodiments described herein, the nAChR agonist is not a full alpha7 receptor agonist. In some embodiments described herein, the nAChR is a full alpha7 receptor agonist.

Administration of a nAChR agonist may desensitize the receptor. Receptor desensitization results in reduced response to an agonist even at higher agonist concentrations, which further results in diminished efficacy of the treatment. For instance, short term desensitization of the nAChR receptor to an agonist may occur over a 24 hour period after administration of the agonist. The potential for receptor desensitization may potentially limit the dosing frequency over a period of time in order to preserve an effective response to the agonist. The methods described herein, provide that the efficacy of nAChR agonists can be surprisingly improved by combining a nAChR agonist with a nAChR PAM. Such combinations are highly efficient for improving the efficacy of a nAChR agonist for treatment of dry eye disease, ocular discomfort, or increasing tear production, when compared to administration of a nAChR agonist alone.

PAMs, as described herein, may bind to an allosteric site on the nAChR and positively allosterically modulate the receptor's activity in the presence of the physiological ligand, acetylcholine (ACh), or another nAChR agonist described herein, or and/or act alone as agonists or antagonists of the receptor through the allosteric binding site. In some embodiments, the PAMs do not act alone as agonists and/or antagonists of nAChR.

One example of measuring potentiation is disclosed in Example 1, which describes an assay evaluating nAChR agonists and PAMs in alpha4beta2 nAchRs expressed in Xenopus laevis (African clawed frog) oocytes.

Considerations for selecting a PAM include, but not limited to, toxicity, side effects from its use, and the ability to exist in a physical form that is stable and easily formulated. Another consideration is the selectivity of the PAM for a particular nAChR subtype. A PAM that is selective for a receptor comprising a particular peripheral nAChR subtype has a functional affinity to such a receptor to a higher degree compared to other peripheral nAChR subtype. Selectivity may be associated with at least a 5-fold affinity difference in EC50 value, at least a 10-fold affinity difference in EC50 value, at least a 20-fold affinity difference in EC50 value, or at least a 50-fold affinity difference in EC50 value.

PAMs may potentiate effects by enhancing the efficacy and or potency of agonists. For instance, an alpha4beta2 selective PAM may selectively enhance effects at, for example, alpha4beta2 peripheral nAChRs over other peripheral nAChR subtypes. Particular peripheral nAChR subtypes include, but are not limited to, alpha3beta4, alpha4beta2, alpha7, alpha3alpha5beta4, and alpha4alpha6beta2.

In some of the embodiments disclosed herein, the PAM may be selective for one or more peripheral nAChR subtypes while showing little affinity for other receptor subtypes. Selectivity for a particular type of receptor subtype may be beneficial if it causes fewer side effects in an individual than a non-selective PAM.

In some of the embodiments described herein, the efficacy of an alpha4beta2 nAChR agonist surprisingly may be improved by combining with an alpha4beta2 nAChR subtype selective PAM. Such combinations are highly efficient for improving the efficacy of an alpha4beta2 agonist for treatment of dry eye disease, ocular discomfort, or increasing tear production, when compared to administration of an alpha4beta2 receptor ligand alone.

In some of the embodiments described herein, the PAM, or a pharmaceutically acceptable salt thereof, is selective for at least one peripheral nAChR subtype selected from alpha3beta4, alpha4beta2, alpha7, and alpha3alpha5beta4. In some of the embodiments described herein, the PAM, or a pharmaceutically acceptable salt thereof, is selective for at least one peripheral nAChR subtype selected from alpha3beta4, alpha4beta2, alpha7, alpha3alpha5beta4, and alpha4alpha6beta2. In some of the embodiments described herein, the PAM, or a pharmaceutically acceptable salt thereof, is selective for at least one peripheral nAChR subtype selected from alpha3beta4, alpha4beta2, alpha3alpha5beta4, and alpha4alpha6beta2.

In some of the embodiments described herein, the PAM, or a pharmaceutically acceptable salt thereof, is selective for one peripheral nAChR subtype. In some of the embodiments described herein, the PAM, or a pharmaceutically acceptable salt thereof, is selective for two peripheral nAChR subtypes. In some of the embodiments described herein, the PAM, or a pharmaceutically acceptable salt thereof, is selective for alpha3beta4. In some of the embodiments described herein, the PAM, or a pharmaceutically acceptable salt thereof, is selective for alpha4beta2. In some of the embodiments described herein, the PAM, or a pharmaceutically acceptable salt thereof, is selective for alpha7. In some of the embodiments described herein, the PAM, or a pharmaceutically acceptable salt thereof, is selective for alpha3alpha5beta4. In some of the embodiments described herein, the PAM, or a pharmaceutically acceptable salt thereof, is selective for alpha4alpha6beta2.

In some of the embodiments described herein, the PAM, or a pharmaceutically acceptable salt thereof, is not selective for alpha7.

Br-PBTC may increase acetylcholine-evoked responses of receptor subtypes alpha2 and alpha4 nAChR and is a subtype selective PAM for these target receptors.

NS-9283 (3-[3-(3-Pyridinyl)-1,2,4-oxadiazol-5-yl]benzonitrile) is a PAM that is selective for alpha4beta2 nAChR.

In some of the embodiments described herein, the PAM alone or in combination with a nAChR agonist selectively binds to at least one of the peripheral nAChR subtype selected from alpha3beta4, alpha4beta2, alpha7, and alpha3alpha5beta4. In some of the embodiments described herein, the PAM alone or in combination with a nAChR agonist selectively binds to at least one peripheral nAChR subtype selected from alpha3beta4, alpha4beta2, alpha 7, alpha3alpha5beta4, and alpha4alpha6beta2. In some of the embodiments described herein, the PAM alone or in combination with a nAChR agonist selectively binds to at least one peripheral nAChR subtype selected from alpha3beta4, alpha4beta2, alpha3alpha5beta4, and alpha4alpha6beta2. In some of the embodiments described herein, the PAM alone or in combination with a nAChR agonist selectively binds to the peripheral nAChR subtype alpha3beta4. In some of the embodiments described herein, the PAM alone or in combination with a nAChR agonist selectively binds to the peripheral nAChR subtype alpha4beta2. In some of the embodiments described herein, the PAM alone or in combination with a nAChR agonist selectively binds to the peripheral nAChR subtype alpha7. In some of the embodiments described herein, the PAM alone or in combination with a nAChR agonist selectively binds to the peripheral nAChR subtype alpha3alpha5beta4. In some of the embodiments described herein, the PAM alone or in combination with a nAChR agonist selectively binds to the peripheral nAChR subtype alpha4alpha6beta2. In some of the embodiments described herein, the PAM alone or in combination with a nAChR agonist does not selectively bind to the peripheral nAChR subtype alpha7.

In some of the embodiments described herein, the PAM is selected from the group consisting of 17-beta-Estradiol, Br-PBTC, ivermectin, galantamine, genistein, 5-hydroxyindole, 4BP-TQS, A-86774, CCMI, levamisole, morantel, LY-2087101, mecamylamine, menthol, NS206, NS1738, NS9283, PNU-120596, RO5126946, TBS-345, dFBR, and HEPES, or a pharmaceutically acceptable salt of any of the foregoing. The chemical name represented by each of the code name is recited below, and may be used interchangeably herein.

| | |
|---|---|
| Br-PBTC | (R)-7-bromo-N-(piperidin-3-yl)benzo[b]thiophene-2-carboxamide |
| NS9283 | 3-[3-(3-Pyridinyl)-1,2,4-oxadiazol-5-yl]benzonitrile |
| 4BP-TQS | 4-(4-Bromophenyl)-3a,4,5,9b-tetrahydro-3H-cyclopenta[c]quinoline-8- |
| | sulfonamide |
| A-86774 | 4-[5-(4-Chlorophenyl)-2-methyl-3-(1-oxopropyl)-1H-pyrrol-1- |
| | yl]benzenesulfonamide |
| CCMI | [N-(4-Chlorophenyl)]-α-[(4-chlorophenyl)-aminomethylene]-3-methyl-5- |
| | isoxazoleacetamide |
| LY-2087101 | [2-[(4-Fluorophenyl)amino]-4-methyl-5-thiazolyl]-3-thienylmethanone |
| NS206 | 3-N-Benzyloxy-3-hydroxyimino-2-oxo-6,7,8,9-tetrahydro-1H- |
| | benzo[g]indole-5-sulfonamide |
| NS1738 | N-(5-Chloro-2-hydroxyphenyl)-N′-[2-chloro-5- |
| | (trifluoromethyl)phenyl]urea |
| PNU-120596 | N-(5-Chloro-2,4-dimethoxyphenyl)-N′-(5-methyl-3-isoxazolyl)-urea |
| R05126946 | (5-chloro-N-[(1S,3R)-2,2-dimethyl-3-(4-sulfamoyl-phenyl)-cyclopropyl]- |
| | 2-methoxy-benzamide) |
| TBS-345 | TBS-345, 4-(3-(4-bromophenyl)-5-phenyl-1H-1,2,4-triazol-1- |
| | yl)benzenesulfonamide |
| dFBR | 6-Bromo-2-(1,1-dimethyl-2-propenyl)-N-1H-indole-3-ethanamine |
| | hydrochloride |
| HEPES | 4-(2-Hydroxyethyl)piperazine-1-ethanesulfonic acid |

In some of the embodiments described herein, the PAM is (R)-7-bromo-N-(piperidin-3-yl)benzo[b]thiophene-2-carboxamide (Br-PBTC) or 3-[3-(3-Pyridinyl)-1,2,4-oxadiazol-5-yl]benzonitrile (NS9283), or a pharmaceutically acceptable salt of either of the foregoing.

In some of the embodiments described herein, the PAM is (R)-7-bromo-N-(piperidin-3-yl)benzo[b]thiophene-2-carboxamide (Br-PBTC), or a pharmaceutically acceptable salt thereof. In some of the embodiments described herein, the PAM is 3-[3-(3-Pyridinyl)-1,2,4-oxadiazol-5-yl]benzonitrile (NS9283), or a pharmaceutically acceptable salt thereof.

Nicotinic acetylcholine receptors may be referred to as nAChRs interchangeably throughout this disclosure. nAChR agonists bind to a particular receptor, activating the receptor to produce the associated biological response.

A nAChR agonist may be characterized as a full or partial agonist as determined by its ability to activate a given receptor to produce a response as compared to the response at that receptor for acetylcholine (ACh). In general, a nAChR agonist is a full agonist if it evokes a response upon binding to a given receptor that is equal or greater to that of ACh. A nAChR agonist is a partial agonist if it evokes a lower response upon binding to the receptor as compared to the response generated from ACh.

nAChR agonist response, from which receptor activation can be determined can, for example, be generated using an appropriate cell-based assay. Cells designed to express a particular nAChR receptor subtype and generate an electrical current response when bound to and activated by a nAChR agonist can be used to characterize the agonist profile of a compound and the amount of receptor activation thus determined. An example of a generic protocol is described below.

Cells that express a particular human nAChR subtype are first exposed to ACh. ACh binds and activates the receptor, thereby evoking a current. The concentration of the ACh is chosen to elicit the maximum response of the receptor (e.g., 1280 micromolar ACh). This current is recorded as the ACh response and serves as the 100% nAChR agonist response and to which responses to other nAChR agonists are compared. After washing, the cells are exposed to a nAChR agonist at various concentrations (e.g., 0.1, 0.3, 1, 3, 10, 30, 100, and 300 micromolar). The current evoked by exposure to the nAChR agonist is measured and recorded for each nAChR concentration. This nAChR agonist response data is then normalized to unity versus the maximal ACh evoked current and plotted as a function of the logarithm of the nAChR agonist concentration. The nAChR agonist response is then calculated as a percentage of the ACh response.

In some cases, the method to determine the relative agonist activity of nAChR agonist comprises conditions wherein the ACh response is evoked from a 1 or more millimolar ACh solution.

A nAChR agonist evoking a response equal to or greater than the maximum ACh response determined at the same receptor type is a full agonist. In some cases, a nAChR agonist evoking a response of less than 100% of the ACh response may still be characterized a full agonist, taking into account experimental variability. For example, variability between tests or measurement methods, and statistical error, may account for differences in the response results. In some cases, a nAChR agonist evoking 80% to 120% of the ACh response is considered a full agonist. In some cases, a nAChR agonist evoking 99% of the ACh response or greater is considered a full agonist. In some cases, a nAChR agonist evoking 95% of the ACh response or greater is considered a full agonist. In some cases, a nAChR agonist evoking 90% of the ACh response or greater is considered a full agonist. In some cases, a nAChR agonist evoking 85% of the ACh response or greater is considered a full agonist. In some cases, a nAChR agonist evoking 80% of the ACh response or greater is considered a full agonist.

Taking into account experimental variability, if the nAChR agonist evokes less than 100% of the ACh response, then generally the agonist is considered a partial agonist. In some cases, a nAChR agonist evoking less than 95% of the ACh response is considered a partial agonist. In some cases, a nAChR agonist evoking less than 90% of the ACh response is considered a partial agonist. In some cases, a nAChR agonist evoking less than 85% of the ACh response is considered a partial agonist. In some cases, a nAChR agonist evoking less than 80% of the ACh response is considered a partial agonist.

In some cases, a nAChR agonist evoking 5% to 95% of the ACh response is considered a partial agonist. In some cases, a nAChR agonist evoking 5% to 90% of the ACh response is considered a partial agonist. In some cases, a nAChR agonist evoking 5% to 85% of the ACh response is considered a partial agonist. In some cases, a nAChR agonist evoking 5% to 80% of the ACh response is considered a partial agonist.

In some cases, a nAChR agonist evoking 10% to 95% of the ACh response is considered a partial agonist. In some cases, a nAChR agonist evoking 10% to 90% of the ACh response is considered a partial agonist. In some cases, a nAChR agonist evoking 10% to 85% of the ACh response is considered a partial agonist. In some cases, a nAChR agonist evoking 10% to 80% of the ACh response is considered a partial agonist.

nAChR agonists that generate a low level of electrical activity at relatively high concentrations of agonist may be described as a weak partial agonist. In some cases, a nAChR agonist evoking 30% or less of the ACh response is considered a weak partial agonist. In some cases, a nAChR agonist evoking 25% or less of the ACh response is considered a weak partial agonist. In some cases, a nAChR agonist evoking 20% or less of the ACh response is considered a weak partial agonist. In some cases, the relatively high concentration of nAChR agonist is at least 100 micromolar. In some cases, the relatively high concentration of nAChR agonist is at least 200 micromolar. In some cases, the relatively high concentration of nAChR agonist is at least 300 micromolar or greater. For instance, a 300 micromolar concentration of nAChR agonist that evokes 25% of the maximal Ach-evoked current is considered a weak partial agonist.

In some embodiments, the nAChR agonist is a full agonist. In some embodiments, the nAChR agonist is a partial agonist. In some embodiments, the nAChR agonist is a weak partial agonist.

In some embodiments, the nAChR agonist, or a pharmaceutically acceptable salt thereof, is an agonist of at least one of the nAChR subtypes selected from alpha3beta4, alpha3alpha5beta4, alpha4beta2, and alpha4alpha6beta2. In some embodiments, the nAChR agonist, or a pharmaceutically acceptable salt thereof, is an agonist of at least two of the nAChR subtypes selected from alpha3beta4, alpha3alpha5beta4, alpha4beta2, and alpha4alpha6beta2. In some embodiments, the nAChR agonist, or a pharmaceutically acceptable salt thereof, is an agonist of at least three of the nAChR subtypes selected from alpha3beta4, alpha3alpha5beta4, alpha4beta2, and alpha4alpha6beta2. In some embodiments, the nAChR agonist, or a pharmaceutically acceptable salt thereof, is an agonist of nAChR subtype alpha3beta4. In some embodiments, the nAChR agonist, or a pharmaceutically acceptable salt thereof, is an agonist of nAChR subtype alpha3alpha5beta4. In some embodiments, the nAChR agonist, or a pharmaceutically acceptable salt thereof, is an agonist of nAChR subtype alpha4beta2. In some embodiments, the nAChR agonist, or a pharmaceutically acceptable salt thereof, is an agonist of nAChR subtype alpha4alpha6beta2. In some embodiments, the nAChR agonist, or a pharmaceutically acceptable salt thereof, is an agonist of nAChR subtype alpha7. In some embodiments, the nAChR agonist, or a pharmaceutically acceptable salt thereof, is not an agonist of nAChR subtype alpha7. In some embodiments, the nAChR agonist, or a pharmaceutically acceptable salt thereof, is not a full agonist of nAChR subtype alpha7. In some embodiments, the nAChR agonist is a full agonist of the aforementioned subtypes. In some embodiments, the nAChR agonist is a partial agonist of the aforementioned subtypes. In some embodiments, the nAChR agonist is a weak partial agonist of the aforementioned subtypes.

In some embodiments, the nAChR agonist, or a pharmaceutically acceptable salt thereof, selectively binds to at least one of the nAChR subtypes selected from alpha3beta4, alpha3alpha5beta4, alpha4beta2, and alpha4alpha6beta2. In some embodiments, the nAChR agonist, or a pharmaceutically acceptable salt thereof, selectively binds to nAChR subtype alpha3beta4. In some embodiments, the nAChR agonist, or a pharmaceutically acceptable salt thereof, selectively binds to nAChR subtype alpha3alpha5beta4. In some embodiments, the nAChR agonist, or a pharmaceutically acceptable salt thereof, selectively binds to nAChR subtype alpha4beta2. In some embodiments, the nAChR agonist, or a pharmaceutically acceptable salt thereof, selectively binds to nAChR subtype alpha4alpha6beta2. In some embodiments, the nAChR agonist, or a pharmaceutically acceptable salt thereof, selectively binds to nAChR subtype alpha7. In some embodiments, the nAChR agonist, or a pharmaceutically acceptable salt thereof, does not selectively bind to nAChR subtype alpha7.

The nAChR agonists contemplated in this disclosure include varenicline, a pharmaceutically acceptable salt thereof, and compound 1, or a pharmaceutically acceptable salt thereof. In some embodiments the nAChR agonist is not varenicline.

Varenicline is characterized as a full agonist of the nAChR subtype alpha7 and a partial agonist of subtypes alpha3beta4, alpha4beta2, alpha6beta2, alpha3alpha5beta4, and alpha4alpha6beta2. It has the potential to activate the trigeminal-parasympathetic pathway, stimulating the production of all three layers of tear film, and potentially serve as a novel candidate to improve the signs and symptoms of dry eye disease. In some of the embodiments described herein, the nAChR agonist is varenicline, or a pharmaceutically acceptable salt thereof. Pharmaceutically acceptable salts of varenicline include varenicline tartrate. Patent related information for varenicline may be found in U.S. Pat. Nos. 9,504,644, 9,504,645, 9,532,944, and 9,597,284.

Compound 1, as recited herein, refers to the structure:

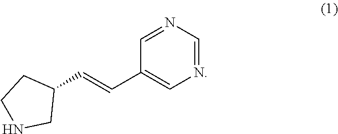 [SMILES: C(=C\[C@H]1CCNC1)/c1cncnc1]

An alternative structural representation of compound 1 is shown here:

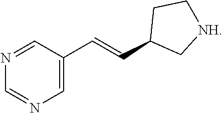 [SMILES: C(=C\[C@H]1CCNC1)/c1cncnc1]

Compound 1 may be also referred to by its chemical name. For instance, compound 1 is also referred to as (R)-5-((E)-2-pyrrolidin-3-ylvinyl)pyrimidine, or variations thereof including 5-{(E)-2-[(3R)-pyrrolidin-3-yl]vinyl}pyrimidine and (R,E)-5-((2-pyrrolidine-3-yl)vinyl)pyrimidine.

Compound 1 is a full agonist of nAChR subtypes alpha4beta2, alpha3beta4, alpha3alpha5beta4, and alpha4alpha6beta2. Compound 1 is a full agonist of nAChR subtypes alpha4beta2, and alpha3beta4.

Compound 1 is a partial agonist of subtype alpha3beta2.

Compound 1 is a weak partial agonist of subtype alpha7. In one example, a 300 micromolar concentration of compound 1 citrate evoked only 25% of the maximal ACh-evoked current.

In some of the embodiments described herein, the nAChR agonist may be compound 1, or a pharmaceutically acceptable salt thereof. Pharmaceutically acceptable salts of compound 1 include galactarate (e.g., hemi-galactarate dihydrate) and citrate (e.g., mono-citrate). Patent related information for compound 1 may be found in U.S. Pat. Nos. 7,098,331, 7,714,001, 8,063,068, 8,067,443, 8,604,191, 9,145,396, 9,981,949, 8,633,222, and PCT publication WO 2017/177024.

In some of the embodiments described herein, the nAChR agonist is (R)-5-((E)-2-pyrrolidin-3-ylvinyl)pyrimidine, or a pharmaceutically acceptable salt thereof. In some of the embodiments described herein, the nAChR agonist is (R)-5-((E)-2-pyrrolidin-3-ylvinyl)pyrimidine hemigalactarate dihydrate. In some of the embodiments described herein, the nAChR agonist is (R)-5-((E)-2-pyrrolidin-3-ylvinyl)pyrimidine mono-citrate.

In some embodiments described herein, the nAChR agonist, or a pharmaceutically acceptable salt thereof, is varenicline, or a pharmaceutically acceptable salt thereof, and the PAM is Br-PBTC, or a pharmaceutically acceptable salt thereof. In some embodiments described herein, the nAChR agonist, or a pharmaceutically acceptable salt thereof, is varenicline, or a pharmaceutically acceptable salt thereof, and the PAM is NS-9283, or a pharmaceutically acceptable salt thereof. In some embodiments described herein, the nAChR agonist, or a pharmaceutically acceptable salt thereof, is compound 1, or a pharmaceutically acceptable salt thereof, and the PAM is Br-PBTC, or a pharmaceutically acceptable salt thereof. In some embodiments described herein, the nAChR agonist, or a pharmaceutically acceptable salt thereof, is compound 1, or a pharmaceutically acceptable salt thereof, and the PAM is NS-9283, or a pharmaceutically acceptable salt thereof.

### Trigeminal-Parasympathetic Pathway

The characterization of the trigeminal parasympathetic pathway and its importance to methods described herein are briefly discussed here. The trigeminal nerve comprises the ophthalmic nerve, the maxillary nerve, and the mandibular nerve. The ophthalmic and maxillary branches of the trigeminal nerve form the afferent paths. The nasal mucosal epithelium and nociceptive nerves are lined with receptors that initiate the afferent limbs of reflexes within the nose. The efferent paths of the reflex proceed from the superior salivary nucleus along the facial nerve (intermedius) to the geniculate ganglion and from there through the greater superficial petrosal nerve via the sphenopalatine ganglion to the glands of the lacrimal functional unit (lacrimal glands, meibomian glands, and goblet cells). Healthy tear film composition is complex and consists of a superficial lipid layer, an intermediary mucin layer, and a deep mucin layer. Each of the components of these layers originates from different glands; the meibomian gland (lipid layer), the lacrimal gland (aqueous layer), and the goblet cells (mucin layer). Neuro-anatomical studies in animals illustrate that the trigeminal-parasympathetic pathway innervates each of these glands. The present disclosure provides methods comprising stimulating the trigeminal-parasympathetic pathway as a means to excrete the tear film components.

In some of the embodiments described herein, the method may comprise activating the nasolacrimal reflex. In some of the embodiments described herein, the method may comprise activating the trigeminal nerve. In some of the embodiments described herein, the method may comprise activating the anterior ethmoidal nerve.

In some of the embodiments described herein, the methods comprise improving tear film homeostasis. In some of the embodiments described herein, the methods comprise re-establishing tear film homeostasis. In some of the embodiments, the individual in need thereof lacks tear film homeostasis or has impaired tear film homeostasis.

### Indicators of Effective Treatment

A variety of tests are available to evaluate an individual's condition before, during, and after treatment with any of the methods disclosed herein. In some of the embodiments disclosed herein, the effective treatment of the individual is indicated by one or more of the tests selected from the group consisting of a) Eye Dryness score test on a visual analog scale, b) Schirmer's test, c) Corneal Fluorescein Staining test, and d) Ocular Surface Disease Index test. Tests to evaluate the signs and symptoms of an ocular condition may be administered under standardized or reproducible conditions in order to obtain an individual's test score. Conditions include exposing the individual to an environment artificially created to adversely challenge the individual or where the environment (temperature, humidity, air flow) is monitored and carefully controlled.

The next sections provide further details on the Eye Dryness Score test on a visual analog scale, Schirmer's test, Corneal Fluorescein Staining test, and Ocular Surface Disease Index test.

### Eye Dryness Score Test

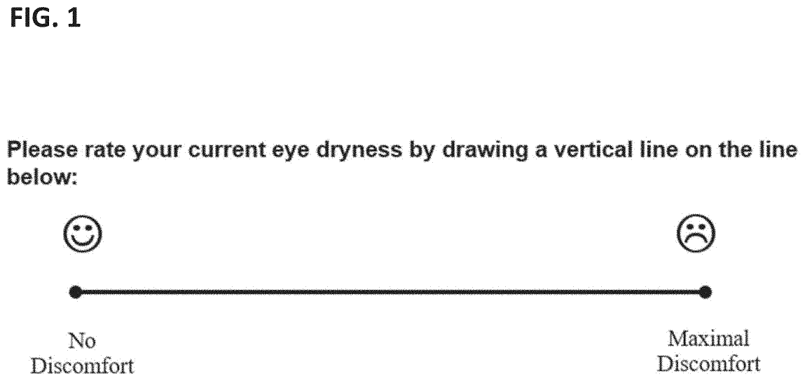

An Eye Dryness Score test on a visual analog scale may be used to evaluate dry eye disease, tearing levels, or ocular discomfort in an individual. The test involves the use of a visual analog scale and comprises a 100 mm horizontal line where one endpoint at 0 is labeled “no discomfort” and the other endpoint at 100 is labeled “maximal discomfort”. FIG. 1 shows an example of the visual analog scale (not shown at actual scale). The individual is asked to rate their ocular symptoms due to eye dryness by placing a vertical mark on the horizontal line to indicate their level of discomfort. The Eye Dryness score is then obtained by identifying where the individual's response is on the 100 mm scale.

The Eye Dryness score, measured in mm, can be used to evaluate the severity of the ocular symptom, and the effectiveness of a particular treatment of an individual. This test has the advantage of being administered to the individual as frequently as every 5 minutes, which allows the test administrator to closely monitor the changes in the individual's symptoms over time. Higher numbers indicate more discomfort for an ocular symptom compared to lower numbers, which indicate relatively lower levels of discomfort. A decrease in the Eye Dryness score is evidence that the treatment is effective in treating dry eye disease, increasing tear production, or improving ocular discomfort. Reduction in Eye Dryness scores over time is evidence of a reduction in or alleviation of the ocular symptom and generally indicates an improvement in the individual's condition.

In some of the embodiments disclosed herein, effective treatment is indicated by a statistically significant decrease in the individual's Eye Dryness score, and wherein the statistically significant decrease in the individual's Eye Dryness score is determined after administration to the individual of the first dose, or the optionally one or more subsequent doses, of the nAChR agonist, or a pharmaceutically acceptable salt thereof, and the first dose, or the optionally one or more subsequent doses, of the PAM, or a pharmaceutically acceptable salt thereof, wherein the individual's Eye Dryness score is compared to a) an Eye Dryness score of the individual prior to administration of the first dose of the nAChR agonist, or a pharmaceutically acceptable salt thereof, and the first dose of the PAM, or a pharmaceutically acceptable salt thereof; b) an Eye Dryness score of an individual administered a control; or c) an Eye Dryness score of an individual administered a comparator compound.

In some of the embodiments disclosed herein, the individual's Eye Dryness score is compared to an Eye Dryness score of the individual prior to administration of the first dose of the nAChR agonist, or a pharmaceutically acceptable salt thereof, and the first dose of the PAM, or a pharmaceutically acceptable salt thereof.

In some of the embodiments disclosed herein, the individual's Eye Dryness score is compared to an Eye Dryness score of an individual administered a control.

In some of the embodiments disclosed herein, the individual's Eye Dryness score is compared to an Eye Dryness score of an individual administered a comparator compound.

In some of the embodiments described herein, the statistically significant decrease in the individual's Eye Dryness Score is at least 5%, 10%, 15%, 20%, 25%, 50%, 75%, 80%, 90%, 95%, 100%, 125%, 150%, 200%, 250%, 300%, or 350%.

In some of the embodiments described herein, the statistically significant decrease in the individual's Eye Dryness score is at least 3 mm, at least 5 mm, at least 10 mm, at least 15 mm, at least 20 mm, at least 25 mm, at least 30 mm, at least 35 mm, at least 40 mm, at least 45 mm, or at least 50 mm.

In some of the embodiments described herein, the statistically significant decrease in the individual's Eye Dryness score is between 3 mm and 10 mm, between 3 mm and 20 mm, between 3 mm and 25 mm, between 3 mm and 30 mm, between 3 mm and 35 mm, between 3 mm and 40 mm, between 3 mm and 45 mm, between 3 mm and 50 mm, between 5 mm and 10 mm, between 5 mm and 20 mm, between 5 mm and 25 mm, between 5 mm and 30 mm, between 5 mm and 35 mm, between 5 mm and 40 mm, between 5 mm and 45 mm, between 5 mm and 50 mm, between 10 mm and 15 mm, between 10 mm and 20 mm, between 10 mm and 25 mm, between 10 mm and 30 mm, between 10 mm and 35 mm, between 10 mm and 40 mm, between 10 mm and 45 mm, between 10 mm and 50 mm, between 15 mm and 20 mm, between 20 mm and 30 mm, between 25 mm and 35 mm, between 30 mm and 40 mm, between 30 mm and 45 mm, or between 30 mm and 50 mm.

In some of the embodiments described herein, the statistically significant decrease in the individual's Eye Dryness Score is characterized by a p value of 0.05 or less, 0.01 or less, 0.005 or less, or 0.001 or less. In some of the embodiments described herein, the statistically significant decrease in the individual's Eye Dryness Score is characterized by a p value of 0.05 or less. In some of the embodiments described herein, the statistically significant decrease in the individual's Eye Dryness Score is characterized by a p value of 0.01 or less.

In some of the embodiments described herein, the statistically significant decrease in the individual's Eye Dryness Score is within 5 minutes, within 10 minutes, within 15 minutes, within 20 minutes, within 30 minutes, within 45 minutes or within 60 minutes of administration of the first dose of an effective amount of the nAChR, or a pharmaceutically acceptable salt thereof, and, optionally, the first dose of the PAM, or a pharmaceutically acceptable salt thereof. In some of the embodiments described herein, the statistically significant decrease in the individual's Eye Dryness Score is within 5 minutes or 10 minutes of the first dose of an effective amount of the nAChR, or a pharmaceutically acceptable salt thereof.

In some of the embodiments described herein, wherein the statistically significant decrease in the individual's Eye Dryness score is based on the individual's Eye Dryness score determined after administering the first dose of the nAChR agonist, or a pharmaceutically acceptable salt thereof.

In some of the embodiments described herein, the statistically significant decrease is based on the individual's Eye Dryness score determined after administering one or more subsequent doses of the nAChR agonist, or a pharmaceutically acceptable salt thereof, and optionally the first or the one or more subsequent doses of the PAM, or a pharmaceutically acceptable salt thereof

### Schirmer Score Test

Dry eye disease affects tear volume and tear production. A Schirmer's test may be used to evaluate tear production and assess the severity of dry eye disease, insufficient tearing, or ocular discomfort in an individual. The test measures the amount of tears produced in each eye. The test typically involves first placing an anesthetic into one or both of the individual's eyes. These drops prevent the eyes from watering in reaction to the test strips. Then, the test administrator places a piece of filter paper inside one or both lower eyelids and the person closes their eyes. After 5 minutes, the test administrator removes the filter paper and assesses how far the tears have travelled on the paper. The Schirmer's test may be administered to one or both eyes.

In general, the smaller the amount of moisture on the paper, the fewer tears that person has produced. In healthy eyes, each strip of paper typically contains more than 10 millimeters of moisture. A Schirmer's score of less than 10 millimeters of moisture may indicate one or more of the following conditions including dry eye disease, abnormally low tearing, or ocular discomfort.

A Schirmer's test may be used to evaluate the effectiveness of a particular treatment in an individual and may be administered multiple times to monitor any change in the severity of the individual's symptoms over a period of time. An increase in Schirmer's scores over time in an individual being treated for dry eye disease, insufficient tearing, or ocular discomfort is evidence for an increase in tear volume or tear production and generally indicates improvement in the individual's condition. An increase in the Schirmer's score over time is evidence that treatment is effective in treating dry eye disease, increasing tear production, or improving ocular discomfort.

In some of the embodiments disclosed herein, effective treatment is indicated by a statistically significant increase in the individual's Schirmer's score, and wherein the statistically significant increase in the individual's Schirmer's score is determined after administration to the individual of the first dose, or the optionally one or more subsequent doses, of the nAChR agonist, or a pharmaceutically acceptable salt thereof, and the first dose, or the optionally one or more subsequent doses, of the PAM, or a pharmaceutically acceptable salt thereof, wherein the individual's Schirmer's score is compared to a) a Schirmer's score of the individual prior to administration of the first dose of the nAChR agonist, or a pharmaceutically acceptable salt thereof, and the first dose of the PAM, or a pharmaceutically acceptable salt thereof b) a Schirmer's score of an individual administered a control; or c) a Schirmer's score of an individual administered a comparator compound.

In some of the embodiments disclosed herein, the individual's Schirmer's score is compared to a Schirmer's score of the individual prior to administration of the first dose of the nAChR agonist, or a pharmaceutically acceptable salt thereof, and the first dose of the PAM, or a pharmaceutically acceptable salt thereof. In some of the embodiments disclosed herein, the individual's Schirmer's score is compared to a Schirmer's score of an individual administered a control. In some of the embodiments disclosed herein, the individual's Schirmer's score is compared to a Schirmer's score of an individual administered a comparator compound.

In some of the embodiments described herein, the statistically significant increase in the individual's Schirmer's score is at least 5%, 10%, 15%, 20%, 25%, 50%, 75%, 80%, 90%, 95%, 100%, 125%, 150%, 200%, 250%, 300%, or 350%. In some of the embodiments described herein, the increase in the individual's Schirmer's score is at least 100%, 200%, or 300%.

In some of the embodiments described herein, the statistically significant increase in the individual's Schirmer's score is at least 3 mm, at least 5 mm, at least 10 mm, at least 15 mm, at least 20 mm, at least 25 mm, at least 30 mm, at least 35 mm, at least 40 mm, at least 45 mm, or at least 50 mm.

In some of the embodiments described herein, the statistically significant increase in the individual's Schirmer's score is between 3 mm and 5 mm, between 3 mm and 10 mm, between 3 mm and 15 mm, between 3 mm and 20 mm, between 3 mm and 25 mm, between 3 mm and 30 mm, between 5 mm and 10 mm, between 5 mm and 15 mm, between 5 mm and 20 mm, between 5 mm and 25 mm, between 5 mm and 30 mm, between 10 mm and 15 mm, between 10 mm and 20 mm, between 10 mm and 25 mm, between 10 mm and 30 mm, 15 mm and 20 mm, between 15 mm and 25 mm, between 15 mm and 30 mm, between 20 mm and 25 mm, or between 20 mm and 30 mm.

In some of the embodiments described herein, the statistically significant increase in the individual's Schirmer's score is characterized by a p value of 0.05 or less, 0.01 or less, 0.005 or less, or 0.001 or less.

In some of the embodiments described herein, the statistically significant decrease in the individual's Schirmer's score is within 5 minutes, within 10 minutes, within 15 minutes, within 20 minutes, within 30 minutes, within 45 minutes or within 60 minutes of administration of the first dose of an effective amount of the nAChR, or a pharmaceutically acceptable salt thereof, and, optionally, the first dose of the PAM, or a pharmaceutically acceptable salt thereof. In some of the embodiments described herein, the statistically significant decrease in the individual's Schirmer's score is within 5 minutes of the first dose of an effective amount of the nAChR, or a pharmaceutically acceptable salt thereof.

In some of the embodiments described herein, wherein the statistically significant decrease in the individual's Schirmer's score is based on the individual's Schirmer's score determined after administering the first dose of the nAChR agonist, or a pharmaceutically acceptable salt thereof.

In some of the embodiments described herein, the statistically significant decrease is based on the individual's Schirmer's score determined after administering one or more subsequent doses of the nAChR agonist, or a pharmaceutically acceptable salt thereof, and optionally the first or the one or more subsequent doses of the PAM, or a pharmaceutically acceptable salt thereof.

### Corneal Fluorescein Staining Test

Corneal surface changes are associated with insufficient tear flow and excessive dryness, as well as dry eye disease and ocular discomfort. Corneal surface changes may include disruption of the mucin coating protecting the surface epithelial cells, and/or damage to the epithelial cell walls.

Corneal Fluorescein Staining test is a diagnostic test for determining corneal surface health and can indicate areas of damage on the corneal surface. The normal corneal surface does not take up water-soluble dyes instilled into the tear film. However, damaged epithelial cells allow water-soluble dyes to diffuse into the surface cells. Fluorescein, which stains damaged epithelial cells, may be visualized on the corneal surface indicating damage as a result of desiccation on the corneal surface.

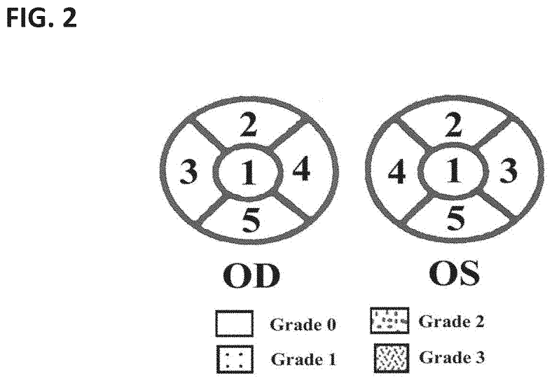

To administer the Corneal Fluorescein Staining test, fluorescein is applied to one or both eyes. Fluorescein is allowed to penetrate and stain the area between surface cells. Devitalized cells and strands of devitalized surface tissue (filaments) can be visualized with this stain. A test administrator then uses a corneal surface scoring system has been developed to rate the severity of damage observed. This scoring system is useful for monitoring dry eye treatment over time. FIG. 2 depicts the NEI/Industry Workshop Scale used in the scoring system. Other equivalent standardized scoring systems may be used. A test administrator grades the areas of the corneal surface that are damaged and calculates the corneal score reflecting the severity of damage to the corneal surface.

A test administrator may use the corneal score to evaluate the effectiveness of a particular treatment in an individual. The test may be administered multiple times to monitor any change in the severity of the individual's ocular surface over a period of time. In general, higher numbers indicate more damage to the corneal surface compared to lower numbers, which indicate lower levels of damage to the corneal surface. Reduction in corneal scores over time is evidence of a reduction in the damage to corneal surface. The decrease in corneal scores generally indicates and improvement in the individual's condition. The decrease in corneal scores over time is also evidence that the treatment is effective in treating dry eye disease, increasing tear production, or improving ocular discomfort.

In some of the embodiments disclosed herein, effective treatment is indicated by a statistically significant decrease in the individual's corneal score, and wherein the statistically significant decrease in the individual's corneal score is determined after administration to the individual of the first dose, or the optionally one or more subsequent doses, of the nAChR agonist, or a pharmaceutically acceptable salt thereof, and the first dose, or the optionally one or more subsequent doses, of the PAM, or a pharmaceutically acceptable salt thereof, wherein the individual's corneal score is compared to a) a corneal score of the individual prior to administration of the first dose of the nAChR agonist, or a pharmaceutically acceptable salt thereof, and the first dose of the PAM, or a pharmaceutically acceptable salt thereof; b) a corneal score of an individual administered a control; or c) a corneal score of an individual administered a comparator compound.

In some of the embodiments disclosed herein, the individual's corneal score is compared to a corneal score of the individual prior to administration of the first dose of the nAChR agonist, or a pharmaceutically acceptable salt thereof, and the first dose of the PAM, or a pharmaceutically acceptable salt thereof. In some of the embodiments disclosed herein, the individual's corneal score is compared to a corneal score of an individual administered a control. In some of the embodiments disclosed herein, the individual's corneal score is compared to a corneal score of an individual administered a comparator compound.

In some of the embodiments described herein, the statistically significant decrease in the individual's corneal score is at least 5%, 10%, 15%, 20%, 25%, 50%, 75%, 80%, 90%, 95%, 100%, 125%, 150%, 200%, 250%, 300%, or 350%.

In some of the embodiments described herein, the statistically significant decrease in the individual's corneal score is characterized by a p value of 0.05 or less, 0.01 or less, 0.005 or less, or 0.001 or less.

In some of the embodiments described herein, the statistically significant decrease in the individual's corneal score is within 5 minutes, within 10 minutes, within 15 minutes, within 20 minutes, within 30 minutes, within 45 minutes or within 60 minutes of administration of the first dose of an effective amount of the nAChR, or a pharmaceutically acceptable salt thereof, and, optionally, the first dose of the PAM, or a pharmaceutically acceptable salt thereof. In some of the embodiments described herein, the statistically significant decrease in the individual's corneal score is within 5 minutes of the first dose of an effective amount of the nAChR, or a pharmaceutically acceptable salt thereof.

In some of the embodiments described herein, wherein the statistically significant decrease in the individual's corneal score is based on the individual's corneal score determined after administering the first dose of the nAChR agonist, or a pharmaceutically acceptable salt thereof.

In some of the embodiments described herein, the statistically significant decrease is based on the individual's corneal score determined after administering one or more subsequent doses of the nAChR agonist, or a pharmaceutically acceptable salt thereof, and optionally the first or the one or more subsequent doses of the PAM, or a pharmaceutically acceptable salt thereof.

### Ocular Surface Disease Index Test

Individuals treated for dry eye disease, increasing tear production, or improving ocular discomfort can provide important information used to diagnose their condition and determine the severity of dry eye symptoms through questionnaires. A well-designed questionnaire can be validated for reproducibility and should consist of relevant questions that elicit responsive answers. One example of a dry-eye questionnaire is the Ocular Surface Disease Index (OSDI), a 12-question survey for dry-eye patients that's been shown to be a reliable and valid instrument for directly assessing symptom frequency. The ocular symptoms that are evaluated include, but are not limited to, burning/stinging, itching, foreign body sensation, eye discomfort, eye dryness, photophobia, and pain. Most people are familiar with questionnaires and understand that they are an efficient way for a health provider to gather information. Questionnaires also reduce bias, as there are no verbal or visual cues to inadvertently influence the respondent. An example of an OSDI questionnaire is shown in f. 3. The test administrator collects the response from the individual and calculates an OSDI based on the individual's answers to the questions.

The OSDI score can be used to evaluate the severity of the ocular symptom, and the effectiveness of a particular treatment of an individual. Higher numbers indicate a higher severity of dry eye disease. Reduction in OSDI scores over time is evidence of a reduction in or alleviation of the ocular symptoms and generally indicates an improvement in the individual's condition. A decrease in the OSDI score is also evidence that the treatment is effective in treating dry eye disease, increasing tear production, or improving ocular discomfort.

In some of the embodiments disclosed herein, effective treatment is indicated by a statistically significant decrease in the individual's OSDI score, and wherein the statistically significant decrease in the individual's OSDI score is determined after administration to the individual of the first dose, or the optionally one or more subsequent doses, of the nAChR agonist, or a pharmaceutically acceptable salt thereof, and the first dose, or the optionally one or more subsequent doses, of the PAM, or a pharmaceutically acceptable salt thereof, wherein the individual's OSDI score is compared to a) an OSDI score of the individual prior to administration of the first dose of the nAChR agonist, or a pharmaceutically acceptable salt thereof, and the first dose of the PAM, or a pharmaceutically acceptable salt thereof b) an OSDI score of an individual administered a control; or c) an OSDI score of an individual administered a comparator compound.

In some of the embodiments disclosed herein, the individual's OSDI score is compared to an OSDI score of the individual prior to administration of the first dose of the nAChR agonist, or a pharmaceutically acceptable salt thereof, and the first dose of the PAM, or a pharmaceutically acceptable salt thereof. In some of the embodiments disclosed herein, the individual's OSDI score is compared to an OSDI score of an individual administered a control. In some of the embodiments disclosed herein, the individual's OSDI score is compared to an OSDI score of an individual administered a comparator compound.

In some of the embodiments described herein, the statistically significant decrease in the individual's OSDI score is at least 5%, 10%, 15%, 20%, 25%, 50%, 75%, 80%, 90%, 95%, 100%, 125%, 150%, 200%, 250%, 300%, or 350%.

In some of the embodiments described herein, the statistically significant decrease in the individual's OSDI score is characterized by a p value of 0.05 or less, 0.01 or less, 0.005 or less, or 0.001 or less.

In some of the embodiments described herein, the statistically significant decrease in the individual's OSDI score is within 5 minutes, within 10 minutes, within 15 minutes, within 20 minutes, within 30 minutes, within 45 minutes or within 60 minutes of administration of the first dose of an effective amount of the nAChR, or a pharmaceutically acceptable salt thereof, and, optionally, the first dose of the PAM, or a pharmaceutically acceptable salt thereof. In some of the embodiments described herein, the statistically significant decrease in the individual's OSDI score is within 5 minutes of the first dose of an effective amount of the nAChR, or a pharmaceutically acceptable salt thereof.

In some of the embodiments described herein, wherein the statistically significant decrease in the individual's OSDI score is based on the individual's OSDI score determined after administering the first dose of the nAChR agonist, or a pharmaceutically acceptable salt thereof.

In some of the embodiments described herein, the statistically significant decrease is based on the individual's OSDI score determined after administering one or more subsequent doses of the nAChR agonist, or a pharmaceutically acceptable salt thereof, and optionally the first or the one or more subsequent doses of the PAM, or a pharmaceutically acceptable salt thereof.

### Maintenance of Effective Treatment Over Time

The present disclosure provides for effective treatment over a period of time where a statistically significant improvement in an individual's score is maintained. The term “maintained” as used in the present disclosure and as it relates to the maintenance of a statistically significant improvement in an individual's score (EDS, Schirmer, corneal, or OSDI) refers to the statistically significant improvement not diminishing below a certain threshold over time. A statistically significant improvement can be maintained even if, at a later point in time, the individual's score changes. An improvement after a first dose of the nAChR, or a pharmaceutically acceptable salt thereof, and, optionally, the first dose of the PAM, or a pharmaceutically acceptable salt thereof, can be maintained without additional dosing or after one or more subsequent doses.

For example, an Eye Dryness score being “maintained within 10% means” that the decrease in individual's Eye Dryness score does not diminish by more than 10% during the specified time. A further improvement in the individual's Eye Dryness score would also be considered maintenance of the statistically significant improvement (e.g., if the Eye Dryness score further improved by 15% during the specified time, this would be considered “maintained within 10%”).

In another example, if there is a statistically significant decrease (improvement) in an individual's Eye Dryness score 30 minutes after taking a first dose of the nAChR, or a pharmaceutically acceptable salt thereof, and, optionally, the first dose of the PAM, or a pharmaceutically acceptable salt thereof, and at a later point, the individual's score is the same or lesser (indicating a benefit to the individual), then the statistically significant improvement is said to be maintained. Alternatively, if at a later point, the individual's Eye Dryness score is greater than the Eye Dryness score 30 minutes after taking a first dose of the nAChR, or a pharmaceutically acceptable salt thereof, and, optionally, the first dose of the PAM, or a pharmaceutically acceptable salt thereof, the individual may still be receiving a therapeutic benefit and the later determined score may still be a statistically significant improvement compared to prior administration of the first dose, or their pre-treatment baseline score.

Note that what constitutes an improvement differs depending on the score being measured. For example, an improvement in an Eye Dryness score, the corneal score and the OSDI score is a decrease in the numerical value of the score. For the Schirmer's test, an improvement is typically an increase in the numerical value of the Schirmer's score.

In some of the embodiments described herein, the maintenance of the statistically significant improvement of the individual's score (e.g., EDS, Schirmer, corneal, or OSDI) means that the statistically significant improvement does not diminish by more than 10%, 20%, 30%, 40%, 50%, or 60%.

In some of the embodiments described herein, the statistically significant improvement in the individual's score (e.g., EDS, Schirmer, corneal, or OSDI) is maintained for at least 30 minutes, at least 45 minutes, at least 60 minutes, at least 2 hours, at least 4 hours, at least 6 hours, at least 8 hours, at least 1 day, at least 1 week, at least 1 month, at least 3 months, at least 6 months, at least 9 months, or at least 12 months. In some of the embodiments described herein, the statistically significant improvement in the individual's score is maintained for at least 30 minutes from administration of the first dose of the nAChR, or a pharmaceutically acceptable salt thereof, and, optionally, the first dose of the PAM, or a pharmaceutically acceptable salt thereof, wherein the statistically significant improvement does not diminish by more than 30%.

In some of the embodiments described herein, the statistically significant improvement in the individual's score (e.g., EDS, Schirmer, corneal, or OSDI) is maintained for at least 30 minutes, at least 45 minutes, at least 60 minutes, at least 2 hours, at least 4 hours, at least 6 hours, at least 8 hours, at least 1 day, at least 1 week, at least 1 month, at least 3 months, at least 6 months, at least 9 months, or at least 12 months after administering the first dose of the nAChR, or a pharmaceutically acceptable salt thereof, and, optionally, the first dose of the PAM, or a pharmaceutically acceptable salt thereof, wherein the statistically significant improvement does not diminish by more than 10%, 20%, 30%, 40%, 50%, or 60% compared to the individual's score within 5 minutes after administering the first dose of the nAChR, or a pharmaceutically acceptable salt thereof, and, optionally, the first dose of the PAM, or a pharmaceutically acceptable salt thereof.

In some of the embodiments described herein, the statistically significant improvement in the individual's score (e.g., EDS, Schirmer, corneal, or OSDI) is maintained for at least 30 minutes, at least 45 minutes, at least 60 minutes, at least 2 hours, at least 4 hours, at least 6 hours, at least 8 hours, at least 1 day, at least 1 week, at least 1 month, at least 3 months, at least 6 months, at least 9 months, or at least 12 months after administering the first dose of the nAChR, or a pharmaceutically acceptable salt thereof, and, optionally, the first dose of the PAM, or a pharmaceutically acceptable salt thereof, wherein the statistically significant improvement does not diminish by more than 20% compared to the individual's corresponding Eye Dryness Score, corneal score, or OSDI score within 5 minutes after administering the first dose of the nAChR, or a pharmaceutically acceptable salt thereof, and, optionally, the first dose of the PAM, or a pharmaceutically acceptable salt thereof.

In some of the embodiments described herein, the statistically significant improvement in the individual's score (e.g., EDS, Schirmer, corneal, or OSDI) is maintained for at least 30 minutes after administering the first dose of the nAChR, or a pharmaceutically acceptable salt thereof, and, optionally, the first dose of the PAM, or a pharmaceutically acceptable salt thereof, wherein the statistically significant improvement does not diminish by more than 10%, 20%, or 30% compared to the individual's score within 5 minutes after administering the first dose of the nAChR, or a pharmaceutically acceptable salt thereof, and, optionally, the first dose of the PAM, or a pharmaceutically acceptable salt thereof.

In some of the embodiments described herein, the individual's Schirmer's score at least 30 minutes, at least 45 minutes, at least 60 minutes, at least 2 hours, at least 4 hours, at least 6 hours, at least 8 hours, at least 1 day, at least 1 week, at least 1 month, at least 3 months, at least 6 months, at least 9 months, or at least 12 months after administering the first dose of the nAChR, or a pharmaceutically acceptable salt thereof, and, optionally, the first dose of the PAM, or a pharmaceutically acceptable salt thereof, does not decrease by more than 10%, 20%, 30%, 40%, 50%, or 60% compared to the individual's corresponding Schirmer's score within 5 minutes after administering the first dose of the nAChR, or a pharmaceutically acceptable salt thereof, and, optionally, the first dose of the PAM, or a pharmaceutically acceptable salt thereof.

In some of the embodiments described herein, the individual's Schirmer's score at least 30 minutes, at least 45 minutes, at least 60 minutes, at least 2 hours, at least 4 hours, at least 6 hours, at least 8 hours, at least 1 day, at least 1 week, at least 1 month, at least 3 months, at least 6 months, at least 9 months, or at least 12 months after administering the first dose of the nAChR, or a pharmaceutically acceptable salt thereof, and, optionally, the first dose of the PAM, or a pharmaceutically acceptable salt thereof, does not decrease by more than 30% compared to the individual's corresponding Schirmer's score within 5 minutes after administering the first dose of the nAChR, or a pharmaceutically acceptable salt thereof, and, optionally, the first dose of the PAM, or a pharmaceutically acceptable salt thereof.

In some of the embodiments described herein, the individual's Schirmer's score at least 30 minutes after administering the first dose of the nAChR, or a pharmaceutically acceptable salt thereof, and, optionally, the first dose of the PAM, or a pharmaceutically acceptable salt thereof, does not decrease by more than 30%, 20%, or 10% compared to the individual's corresponding Schirmer's score within 5 minutes after administering the first dose of the nAChR, or a pharmaceutically acceptable salt thereof, and, optionally, the first dose of the PAM, or a pharmaceutically acceptable salt thereof.

In some of the embodiments described herein, the individual's Eye Dryness score, corneal score, or OSDI score at least 30 minutes, at least 45 minutes, at least 60 minutes, at least 2 hours, at least 4 hours, at least 6 hours, at least 8 hours, at least 1 day, at least 1 week, at least 1 month, at least 3 months, at least 6 months, at least 9 months, or at least 12 months after administering the first dose of the nAChR, or a pharmaceutically acceptable salt thereof, and, optionally, the first dose of the PAM, or a pharmaceutically acceptable salt thereof, does not increase by more than 10%, 20%, 30%, 40%, 50%, or 60% compared to the individual's corresponding Eye Dryness score, corneal score, or OSDI score within 5 minutes after administering the first dose of the nAChR, or a pharmaceutically acceptable salt thereof, and, optionally, the first dose of the PAM, or a pharmaceutically acceptable salt thereof.

In some of the embodiments described herein, the individual's Eye Dryness score, corneal score, or OSDI score at least 30 minutes, at least 45 minutes, at least 60 minutes, at least 2 hours, at least 4 hours, at least 6 hours, at least 8 hours, at least 1 day, at least 1 week, at least 1 month, at least 3 months, at least 6 months, at least 9 months, or at least 12 months after administering the first dose of the nAChR, or a pharmaceutically acceptable salt thereof, and, optionally, the first dose of the PAM, or a pharmaceutically acceptable salt thereof, does not increase by more than 30% compared to the individual's corresponding Eye Dryness score, corneal score, or OSDI score within 5 minutes after administering first dose of the nAChR, or a pharmaceutically acceptable salt thereof, and, optionally, the first dose of the PAM, or a pharmaceutically acceptable salt thereof.

In some of the embodiments described herein, the individual's Eye Dryness score, corneal score, or OSDI score at least 30 minutes after administering first dose of the nAChR, or a pharmaceutically acceptable salt thereof, and, optionally, the first dose of the PAM, or a pharmaceutically acceptable salt thereof, does not increase by more than 30%, 20%, or 10% compared to the individual's corresponding Eye Dryness score, corneal score, or OSDI score within 5 minutes after administering the first dose of the nAChR, or a pharmaceutically acceptable salt thereof, and, optionally, the first dose of the PAM, or a pharmaceutically acceptable salt thereof.

### Humidity

The term “humidity” may refer to relative humidity, which is the ratio of the partial pressure of water vapor to the equilibrium vapor pressure of water at a given temperature. Relative humidity depends on temperature and the pressure of the system of interest. It requires less water vapor to attain high relative humidity at low temperatures; more water vapor is required to attain high relative humidity in warm or hot air. In some of the embodiments described herein, the individual is present in an environment with reduced relative humidity between determinations of the individual's score (Eye dryness, Schirmer's, corneal, OSDI).

Between determinations of an individual's scoring on a particular test, the individual may be present or exposed to an environment artificially created to adversely challenge the individual. For instance, the individual may be in a room where the temperature, humidity, air flow is monitored and controlled to create adverse conditions. In another example, the individual may wear desiccating googles and their eyes may be challenged by low relative humidity.

In some of the embodiments described herein, between determinations of the individual's Eye Dryness Score, between determinations of the individual's Schirmer's score, between determinations of the individual's corneal score, and between determinations of the individual's OSDI score, the individual is present in an environment with reduced humidity. In some of the embodiments described herein, between determinations of the individual's Eye Dryness Score, between determinations of the individual's Schirmer's score, between determinations of the individual's corneal score, and between determinations of the individual's OSDI score, the individual is present in an environment with reduced humidity and a temperature lower or higher than room temperature. In some of the embodiments described herein, the reduced relative humidity at room temperature is less than 40%, less than 30%, less than 20%, less than 10%, or less than 5%. Room temperature is between 15 degrees Celsius (59 degrees Fahrenheit) and 25 degrees Celsius (77 degrees Fahrenheit).

### Dosing Timing and Method of Administration

The schedule of doses administered to an individual depends on various considerations including the duration of effectiveness of each dose, the pharmacokinetic profile of the drug, and the effect of the dose on the body. For example, wherein the patient's condition does not improve, upon the health provider's discretion, the administration of the nAChR agonist, or pharmaceutically acceptable salt thereof, or the PAM, or pharmaceutically acceptable salt thereof, may be increased in frequency or administered chronically in order to ameliorate or otherwise control or limit the symptoms of the patient's disease or condition. In another example, if an individual's condition does improve, upon the health provider's discretion, the frequency of administration of a dose of the nAChR agonist, or pharmaceutically acceptable salt thereof, or a dose of the PAM, or pharmaceutically acceptable salt thereof, may be reduced while maintaining effective treatment of the individual. For instance, the period of time between administrations of one or more doses is extended, or the period of time between days the individual is administered one or more doses is extended. As a non-limiting example, daily administration of one or more doses is modified to administration of one or more doses every other day, or once a week.

The term “dose”, as used herein, may refer to a dose of the nAChR agonist, or pharmaceutically acceptable salt thereof, or a dose of the PAM, or pharmaceutically acceptable salt thereof.

In some of the embodiments described herein, a dose is administered to the individual in need thereof one to four times daily after the first day of administration, once a day after the first day of administration, twice a day after the first day of administration, or three times a day after the first day of administration.

In some embodiments described herein, the individual is administered one or more subsequent doses over time, wherein effective treatment of the individual is maintained even with a reduction in the amount of the nAChR agonist, or pharmaceutically acceptable salt thereof, or the PAM, or pharmaceutically acceptable salt thereof, administered to the individual over time. In some of the embodiments described herein, the dose frequency of the nAChR agonist, or pharmaceutically acceptable salt thereof, or the PAM, or pharmaceutically acceptable salt thereof, administered to the individual is reduced over time. For instance, a dose administered twice a day is reduced to a dose administered once a day.

In some of the embodiments described herein, the length of time between dosing is increased. For instance, administration of a dose every 4 hours is modified to administration of a dose every 8 or 12 hours.

In some of the embodiments described herein, the total amount of nAChR agonist, or a corresponding amount of the pharmaceutically acceptable salt thereof, or the total amount of PAM, or a corresponding amount of the pharmaceutically acceptable salt thereof, per dose is reduced over time. For instance, a dose of 2000 micrograms is reduced to 1000 micrograms, wherein administration of reduced dose maintains the effective treatment of the individual. In some embodiments described herein, the reduced dosage is provided by a reduction in administrations of the nAChR agonist, or pharmaceutically acceptable salt thereof, or the PAM, or pharmaceutically acceptable salt thereof. For instance, where administered as a nasal spray, a dose comprising two administrations or sprays of a nAChR, or a pharmaceutically acceptable salt thereof, is reduced by administering only one spray.

In some of the embodiments described herein, the dose comprises multiple administrations of the nAChR agonist, or pharmaceutically acceptable salt thereof, or the PAM, or pharmaceutically acceptable salt thereof, to each nostril. In some of the embodiments described herein, the dose comprises multiple administrations of the nAChR agonist, or pharmaceutically acceptable salt thereof, or the PAM, or pharmaceutically acceptable salt thereof, to one nostril.

In some of the embodiments described herein, the dose comprises a single administration of the nAChR agonist, or pharmaceutically acceptable salt thereof, or the PAM, or pharmaceutically acceptable salt thereof, to each nostril. In some of the embodiments described herein, the dose comprises a single administration of the nAChR agonist, or pharmaceutically acceptable salt thereof, or the PAM, or pharmaceutically acceptable salt thereof, to one nostril.

In some of the embodiments described herein, the nAChR agonist, or pharmaceutically acceptable salt thereof, or the PAM, or pharmaceutically acceptable salt thereof, is administered to one nostril per dose. In some of the embodiments described herein, the nAChR agonist, or pharmaceutically acceptable salt thereof, or the PAM, or pharmaceutically acceptable salt thereof, is administered to both nostrils per dose.

In some of the embodiments described herein, the nAChR agonist, or pharmaceutically acceptable salt thereof, or the PAM, or pharmaceutically acceptable salt thereof, is administered for at least 28 days. In some of the embodiments described herein, the nAChR agonist, or pharmaceutically acceptable salt thereof, or the PAM, or pharmaceutically acceptable salt thereof, is administered for at least one or more months.

In some of the embodiments described herein, the nAChR agonist, or pharmaceutically acceptable salt thereof, or the PAM, or pharmaceutically acceptable salt thereof, is administered for at least 2 months, at least 3 months, at least 4 months, at least 5 months, at least 6 months, at least 7 months, at least 8 months, at least 9 months, at least 10 months, at least 11 months, or at least one year. In some of the embodiments described herein, the nAChR agonist, or pharmaceutically acceptable salt thereof, or the PAM, or pharmaceutically acceptable salt thereof, is administered for 2-52 weeks, 2-40 weeks, 2-36 weeks, 2-24 weeks, 2-12 weeks, 2-8 weeks, 4-52 weeks, 4-40 weeks, 4-36 weeks, 4-24 weeks, 4-12 weeks, 4-8 weeks, 5-52 weeks, 5-40 weeks, 5-36 weeks, 5-24 weeks, 5-12 weeks, 5-8 weeks, 6-52 weeks, 6-40 weeks, 6-36 weeks, 6-24 weeks, 6-12 weeks, or 6-8 weeks.

In some of the embodiments described herein, the method comprises a first dose and one or more subsequent doses of the effective amount of the nAChR agonist, or pharmaceutically acceptable salt thereof, or the PAM, or pharmaceutically acceptable salt thereof. The one or more subsequent doses are administered after a period of time after the first dose. This period of time between the first dose and the next subsequent dose is at least 1 hour, at least 2 hours, at least 3 hours, at least 4 hours, at least 5 hours, at least 6 hours, at least 7 hours, or at least 8 hours. The period of time between the first dose and the next subsequent dose is between 1-3 hours, 2-4 hours, 3-6 hours, or 4-8 hours. The period of time between the one or more subsequent doses is at least 1 hour, at least 2 hours, at least 3 hours, at least 4 hours, at least 5 hours, at least 6 hours, at least 7 hours, or at least 8 hours. The period of time between the one or more subsequent doses is between 1-3 hours, 2-4 hours, 3-6 hours, or 4-8 hours.

In some embodiments described herein, the PAM, or the pharmaceutically acceptable salt thereof, is administered to the individual in need thereof administered after the nAChR agonist, or the pharmaceutically acceptable salt thereof. In some embodiments described herein, the PAM, or the pharmaceutically acceptable salt thereof, is administered to the individual in need thereof before the nAChR agonist, or the pharmaceutically acceptable salt thereof. In some embodiments described herein, the PAM, or the pharmaceutically acceptable salt thereof, and the nAChR agonist, or the pharmaceutically acceptable salt thereof, are administered to the individual in need thereof at the same time.

In some of the embodiments described herein, the method comprises administering a dose of an effective amount of a PAM, or a pharmaceutically acceptable salt thereof, and a dose of an effective amount of a nAChR agonist, or a pharmaceutically acceptable salt thereof. In some embodiments, for each dose of nAChR agonist administered, a dose of PAM is administered before the nAChR. In some embodiments, for each dose of nAChR agonist administered, a dose of PAM is administered after the nAChR agonist. In some embodiments, for each dose of nAChR agonist administered, a dose of PAM is administered at the same time as the nAChR agonist.

Over the course of chronic treatment, wherein the individual is administered multiple doses over a period of time, the individual may be administered more doses of nAChR, or a pharmaceutically acceptable salt thereof, than doses of PAM, or a pharmaceutically acceptable salt thereof, or vice versa. The ratio of a dose of a nAChR agonist, or the pharmaceutically acceptable salt thereof, and a dose of a PAM, or a pharmaceutically acceptable salt thereof, administered to an individual is not required to be 1:1. The ratio will depend on the dosing regimen prescribed to the individual. In some of the embodiments disclosed herein, the ratio of a dose of a nAChR agonist, or the pharmaceutically acceptable salt thereof, and a dose of a PAM, or a pharmaceutically acceptable salt thereof, administered to an individual is 1:1, 1:2, 1:3, 1:4, 1:5, 1:6, 1:7, 1:8, 1:9, 1:10, 10:1, 9:1, 8:1, 7:1, 6:1, 5:1, 4:1, 3:1, 2:1 over a treatment course.

In some of the embodiments described herein, the method comprises administering a first dose, and one or more subsequent doses, of an effective amount of a PAM, or a pharmaceutically acceptable salt thereof, and a first dose, and one or more subsequent doses, of an effective amount of a nAChR agonist, or a pharmaceutically acceptable salt thereof.

In some embodiments described herein, the time period between administration of a dose of PAM, or a pharmaceutically acceptable salt thereof, and a dose of an effective amount of a nAChR agonist, or a pharmaceutically acceptable salt thereof is less than 5 minutes, between 5-60 minutes, between 30-90 minutes, between 1-3 hours, between 1-8 hours, between 1-12 hours, between 1-24 hours, between 8-12 hours, between 8-24 hours, between 12-24 hours, between 1-3 days, between 1-7 days, between 1-14 days, between 1-28 days, between 3-7 days, between 3-14 days, between 3-28 days, between 7-14 days, or between 7-28 days.

In some of the embodiments described here, the method comprises administering a first dose and one or more subsequent doses of the nAchR agonist, or a pharmaceutically acceptable salt thereof, and a first dose and one or more subsequent doses of the PAM, or a pharmaceutically acceptable salt thereof. The one or more subsequent doses are administered after a period of time after the first dose. This period of time between the first dose and the next subsequent dose is at least 1 hour, at least 2 hours, at least 3 hours, at least 4 hours, at least 5 hours, at least 6 hours, at least 7 hours, or at least 8 hours. The period of time between the first dose and the next subsequent dose is between 1-3 hours, 2-4 hours, 3-6 hours, or 4-8 hours. The period of time between the one or more subsequent doses is at least 1 hour, at least 2 hours, at least 3 hours, at least 4 hours, at least 5 hours, at least 6 hours, at least 7 hours, or at least 8 hours. The period of time between the one or more subsequent doses is between 1-3 hours, 2-4 hours, 3-6 hours, or 4-8 hours.

In some of the embodiments described herein, the PAM, or the pharmaceutically acceptable salt thereof, and the nAChR agonist, or the pharmaceutically acceptable salt thereof, are administered to the individual in need thereof in separate dosage forms. In some of the embodiments described herein, the PAM, or the pharmaceutically acceptable salt thereof, and the nAChR agonist, or the pharmaceutically acceptable salt thereof, are administered to the individual in need thereof in a combined dosage form.

### Dosage Amount and Volumes

In some of the embodiments described herein, the dosages described herein are administered for prophylactic and/or therapeutic treatments. Dosage amounts effective for therapeutic treatments depends on the severity and course of the disease or condition, previous therapy, the patient's health, status, weight, and response to the drugs, and the judgment of the treating health provider. Therapeutically effective amounts are optionally determined by methods including, but not limited to, a dose escalation clinical trial.

In prophylactic applications, the pharmaceutical formulations described herein are administered to a patient susceptible to or otherwise at risk of a particular disease, disorder or condition. Such an amount is defined to be a “prophylactically effective amount or dose.” In this use, the amount will depend on the severity and course of the disease, disorder, or condition, previously therapy, the patient's health status and response to the drugs, and the judgment of the treating health provider.

In some of the embodiments described herein, the amount per dose of the nAChR agonist or the PAM, administered to the individual is 5-4000 micrograms, 5-1000 micrograms, 10-2000 micrograms, 10-700 micrograms, 100-700 micrograms, 100-600 micrograms, 100-500 micrograms, 200-700 micrograms, 200-600 micrograms, 200-500 micrograms, 300-600 micrograms, 300-500 micrograms, 900-4000 micrograms, 900-3000 micrograms, 900-2500 micrograms, 900-2000 micrograms, 1000-4000 micrograms, 1000-2500 micrograms, 1000-2000 micrograms, 1250-4000 micrograms, 1250-2500 micrograms, 1250-2000 micrograms, 1500-4000 micrograms, 1500-3000 micrograms, 1500-2500 micrograms, 1500-2000 micrograms, 1800-4000 micrograms, 1800-3000 micrograms, 1800-2500 micrograms, 1800-2250 micrograms, 2000-4000 micrograms, 2000-3000 micrograms, or 2000-2500 micrograms, or a corresponding amount of a pharmaceutically acceptable salt thereof.

In some of the embodiments described herein, the amount per dose of the nAChR agonist or the PAM administered to the individual is 900-2500 micrograms, 1000-2500 micrograms, 1500-3000 micrograms, 1800-2500 micrograms, 1800-2250 micrograms, or a corresponding amount of a pharmaceutically acceptable salt thereof. In some of the embodiments described herein, the amount per dose of varenicline or compound 1 administered to the individual is 900-2500 micrograms, 1000-2500 micrograms, 1500-3000 micrograms, 1800-2500 micrograms, 1800-2250 micrograms, or a corresponding amount of a pharmaceutically acceptable salt thereof.

In some of the embodiments described herein, the amount per dose of varenicline administered to the individual is 5-15 micrograms, 50-65 micrograms, 100-125 micrograms, or a corresponding amount of a pharmaceutically acceptable salt thereof.

In some of the embodiments described herein, the amount per dose of compound 1 administered to the individual is 150-300 micrograms, 900-1200 micrograms, 2100-2400 micrograms, or a corresponding amount of a pharmaceutically acceptable salt thereof.

In some of the embodiments described herein, the pharmaceutically acceptable salt of the nAChR agonist or the PAM is administered. In some of the embodiments described herein, the free base of the nAChR agonist or the PAM is administered.

The volume of the pharmaceutical formulation administered to an individual depends on various factors, including the route of administration and the type of delivery device. For nasal administration, the volume of the pharmaceutical formulation should be sufficient to deliver the effective amount of the drug to the nasal cavity. Too little a volume might result in the drug not reaching the nasal cavity. On the other hand, the volume should not be so large as to be impractical, uncomfortable, or too difficult to administer to the individual. In addition, too large of a volume may result in the pharmaceutical formulation being delivered to areas of the body not intended for delivery. This can result in waste of the pharmaceutical formulation or irritation of tissues. For instance, reducing a dose volume from 200 microliters to 100 microliters may reduce the incidence of irritation of the upper throat/soft palate by reducing post-nasal drip after instillation.

In some of the embodiments described herein, the nAChR agonist, or a pharmaceutically acceptable salt thereof, or the PAM, or pharmaceutically acceptable salt thereof, is administered in a pharmaceutical formulation for nasal administration, and the total volume of the pharmaceutical formulation per dose is 50-250 microliters, 75-125 microliters, 150-250 microliters, or 175-225 microliters.

In some of the embodiments described herein, the nAChR agonist, or a pharmaceutically acceptable salt thereof, or the PAM, or pharmaceutically acceptable salt thereof, is administered in a pharmaceutical formulation for nasal administration, and the total volume of the pharmaceutical formulation per dose is about 50 microliters, about 75 microliters, about 100 microliters, about 125 microliters, about 150 microliters, about 175 microliters, about 200 microliters, about 225 microliters, or about 250 microliters.

In some of the embodiments described herein, the nAChR agonist, or a pharmaceutically acceptable salt thereof, or the PAM, or pharmaceutically acceptable salt thereof, is administered in a pharmaceutical formulation for nasal administration, and the total volume of the pharmaceutical formulation per nostril is 50-250 microliters, 75-125 microliters, 150-250 microliters, or 175-225 microliters.

In some of the embodiments described herein, the nAChR agonist, or a pharmaceutically acceptable salt thereof, or the PAM, or pharmaceutically acceptable salt thereof, is administered in a pharmaceutical formulation for nasal administration, and the total volume of the pharmaceutical formulation per nostril is about 50 microliters, about 75 microliters, about 100 microliters, about 125 microliters, about 150 microliters, about 175 microliters, about 200 microliters, about 225 microliters, or about 250 microliters.

In some of the embodiments described herein, the nAChR agonist, or a pharmaceutically acceptable salt thereof, or the PAM, or pharmaceutically acceptable salt thereof, is administered in a pharmaceutical formulation for nasal administration comprising between 1 mg/mL and 40 mg/mL, between 1 mg/mL and 30 mg/mL, between 1 mg/mL and 20 mg/mL, between 1 mg/mL and 10 mg/mL, between 1 mg/mL and 5 mg/mL, 2 mg/mL and 40 mg/mL, between 2 mg/mL and 30 mg/mL, between 2 mg/mL and 20 mg/mL, between 2 mg/mL and 10 mg/mL, between 2 mg/mL and 5 mg/mL, 5 mg/mL and 40 mg/mL, between 5 mg/mL and 30 mg/mL, between 5 mg/mL and 20 mg/mL, between 5 mg/mL and 10 mg/mL, or between 5 mg/mL and 15 mg/mL, of the nAChR agonist, or a corresponding amount of a pharmaceutically acceptable salt thereof, or the PAM, or a corresponding amount of a pharmaceutically acceptable salt thereof.

In some of the embodiments described herein, the nAChR agonist, or a pharmaceutically acceptable salt thereof, or the PAM, or pharmaceutically acceptable salt thereof, is administered in a pharmaceutical formulation for nasal administration, wherein the concentration of the nAChR agonist, or PAM, per dose is 0.01% (w/v) to 0.5% (w/v), 0.01% (w/v) to 1.0% (w/v), 0.01% (w/v) to 1.5% (w/v), 0.01% (w/v) to 2.0% (w/v), 0.01% (w/v) to 2.5% (w/v), 0.01% (w/v) to 3.0% (w/v), 0.5% (w/v) to 1.0% (w/v), 0.5% (w/v) to 1.5% (w/v), 0.5% (w/v) to 2.0% (w/v), 0.5% (w/v) to 2.5% (w/v), 0.5% (w/v) to 3.0% (w/v), 1.0% (w/v) to 1.5% (w/v), 1.0% (w/v) to 2.0% (w/v), 1.0% (w/v) to 2.5% (w/v), 1.0% (w/v) to 3.0% (w/v), 1.5% (w/v) to 2.0% (w/v), 1.5% (w/v) to 2.5% (w/v), 1.5% (w/v) to 3.0% (w/v), 2.0% (w/v) to 2.5% (w/v), or 2.0% (w/v) to 3.0% (w/v), or a corresponding amount of a pharmaceutically acceptable salt thereof.

In some of the embodiments disclosed herein, the concentration of nAChR agonist, or pharmaceutically acceptable salt thereof, administered to an individual in need thereof is 10×, 9×, 8×, 7×, 6×, 5×, 4×, 3×, or 2× higher than the concentration of the PAM, or pharmaceutically acceptable salt thereof, administered to the individual in need thereof. In some of the embodiments disclosed herein, the concentration of nAChR agonist, or pharmaceutically acceptable salt thereof, administered to an individual in need thereof is 1× to 2× higher than the concentration of the PAM, or pharmaceutically acceptable salt thereof, administered to the individual in need thereof. In some of the embodiments disclosed herein, the concentration of the PAM, or pharmaceutically acceptable salt thereof, administered to an individual in need thereof is 1× to 2× higher than the concentration of the nAChR agonist, or pharmaceutically acceptable salt thereof, administered to the individual in need thereof.

In some of the embodiments disclosed herein, the amount of nAChR agonist, or pharmaceutically acceptable salt thereof, administered to an individual in need thereof is 10×, 9×, 8×, 7×, 6×, 5×, 4×, 3×, or 2× higher than the amount of the PAM, or pharmaceutically acceptable salt thereof, administered to the individual in need thereof. In some of the embodiments disclosed herein, the amount of nAChR agonist, or pharmaceutically acceptable salt thereof, administered to an individual in need thereof is 1× to 2× higher than the amount of the PAM, or pharmaceutically acceptable salt thereof, administered to the individual in need thereof. In some of the embodiments disclosed herein, the amount of the PAM, or pharmaceutically acceptable salt thereof, administered to an individual in need thereof is 1× to 2× higher than the amount of the nAChR agonist, or pharmaceutically acceptable salt thereof, administered to the individual in need thereof.

In some of the embodiments described herein, varenicline, or a pharmaceutically acceptable salt thereof, or the PAM, or pharmaceutically acceptable salt thereof, is administered in a pharmaceutical formulation for nasal administration, wherein the concentration of varenicline, or PAM, per dose is about 0.058% (w/v), or about 0.12% (w/v), or a corresponding amount of a pharmaceutically acceptable salt thereof.

In some of the embodiments described herein, varenicline, or a pharmaceutically acceptable salt thereof, or the PAM, or pharmaceutically acceptable salt thereof, is administered in a pharmaceutical formulation for nasal administration, wherein the concentration of varenicline, or PAM, per dose is less than about 0.06% (w/v), or less than 0.15% (w/v), or a corresponding amount of a pharmaceutically acceptable salt thereof.

In some of the embodiments described herein, varenicline, or a pharmaceutically acceptable salt thereof, or the PAM, or pharmaceutically acceptable salt thereof, is administered in a pharmaceutical formulation for nasal administration, wherein the concentration of varenicline, or PAM, per dose is about 0.058% (w/v), or about 0.12% (w/v), or a corresponding amount of a pharmaceutically acceptable salt thereof; and wherein the volume per dose is about 50 microliters.

In some of the embodiments described herein, varenicline, or a pharmaceutically acceptable salt thereof, or the PAM, or pharmaceutically acceptable salt thereof, is administered in a pharmaceutical formulation for nasal administration, wherein the concentration of varenicline, or PAM, per dose is less than about 0.06% (w/v), or less than 0.15% (w/v), or a corresponding amount of a pharmaceutically acceptable salt thereof; and wherein the volume per dose is about 50 microliters.

In some of the embodiments described herein, varenicline, or a pharmaceutically acceptable salt thereof, or the PAM, or pharmaceutically acceptable salt thereof, is administered in a pharmaceutical formulation for nasal administration, wherein the concentration of varenicline, or PAM, per dose is about 0.058% (w/v), or about 0.12% (w/v), or a corresponding amount of a pharmaceutically acceptable salt thereof; and wherein the volume per dose is about 100 microliters. In some of the embodiments herein, the 100 microliter dose is delivered as two 50 microliter sprays. In some of the embodiments herein, the 100 microliter dose is delivered as a single 100 microliter spray.

In some of the embodiments described herein, varenicline, or a pharmaceutically acceptable salt thereof, or the PAM, or pharmaceutically acceptable salt thereof, is administered in a pharmaceutical formulation for nasal administration, wherein the concentration of varenicline, or PAM, per dose is less than about 0.06% (w/v), or less than 0.15% (w/v), or a corresponding amount of a pharmaceutically acceptable salt thereof; and wherein the volume per dose is about 100 microliters. In some of the embodiments herein, the 100 microliter dose is delivered as two 50 microliter sprays. In some of the embodiments herein, the 100 microliter dose is delivered as a single 100 microliter spray.

In some of the embodiments described herein, the nAChR or the PAM is a pharmaceutically acceptable salt. In some of the embodiments described herein, the nAChR is compound 1 galactarate (e.g., hemi-galactarate dihydrate) or compound 1 citrate (e.g., a mono-citrate salt). In some of the embodiments described herein, compound 1 is a free base. In some of the embodiments described herein, the nAChR agonist is varenicline tartrate.

The amount of nAChR agonist free base and the corresponding amount of salt administered to an individual in need thereof may be calculated based on the nAChR agonist concentration of the pharmaceutical formulation and volume of the pharmaceutical formulation administered to the individual need thereof. The following examples illustrate the relationship between concentration and volume of the pharmaceutical formulation, and the amounts of nAChR agonist salt and free base administered to the individual.

For example, a 2.0% compound 1 hemi-galactarate dihydrate solution is equivalent to 20 mg of the salt per 1 mL of solution. The 2.0% compound 1 hemi-galactarate dihydrate solution corresponds to a 1.1% compound 1 free base solution. 1.1% of free base solution is equivalent to 11.1 mg/mL of compound 1 free base.

50 μL of 2.0% compound 1 hemi-galactarate dihydrate solution contains about 1000 μg of compound 1 hemi-galactarate dihydrate, which is equivalent to about 554 μg of compound 1 free base. Likewise, 100 μL of 2.0% compound 1 hemi-galactarate dihydrate solution contains about 2000 μg of compound 1 hemi-galactarate dihydrate, which is equivalent to about 1108 μg of compound 1 free base. Amounts of salt and the corresponding amount of free base may be calculated for other concentrations or volumes in a similar fashion.

In another example, a 2.3% compound 1 mono-citrate solution is equivalent to 23.2 mg of the salt per 1 mL of solution. The 2.3% compound 1 mono-citrate solution corresponds to a 1.1% compound 1 free base solution. 1.1% of free base solution is equivalent to 11.1 mg/mL of compound 1 free base.

50 μL of 2.3% compound 1 mono-citrate solution contains about 1161 μg of compound 1 mono-citrate, which is equivalent to about 554 μg of compound 1 free base. Likewise, 100 μL of 2.3% compound 1 mono-citrate solution contains about 2322 μg of compound 1 mono-citrate, which is equivalent to about 1108 μg of compound 1 free base. Amounts of salt and the corresponding amount of free base may be calculated for other concentrations or volumes in a similar fashion.

In another example, a 0.10% varenicline tartrate solution is equivalent to 1.00 mg of the salt per 1 mL of solution. The 0.10% varenicline tartrate solution corresponds to 0.0585% varenicline free base in solution. 0.0585% of free base solution is equivalent to 0.584 mg/mL of varenicline free base.

50 μL of 0.10% varenicline tartrate solution contains about 50 μg of varenicline tartrate, which is equivalent to about 29.2 μg of varenicline free base. Likewise, 100 μL of 0.10% varenicline tartrate solution contains about 100 μg of varenicline tartrate, which is equivalent to about 58.5 μg of varenicline free base. Amounts of salt and the corresponding amount of free base may be calculated for other concentrations or volumes in a similar fashion.

In another example, a 0.20% varenicline tartrate solution is equivalent to 2.00 mg of the salt per 1 mL of solution. The 0.20% varenicline tartrate solution corresponds to 0.117% varenicline free base in solution. 0.117% of free base solution is equivalent to 1.17 mg/mL of varenicline free base.

50 μL of 0.20% varenicline tartrate solution contains about 100 μg of varenicline tartrate, which is equivalent to about 58.5 μg of varenicline free base. Likewise, 100 μL of 0.20% varenicline tartrate solution contains about 200 μg of varenicline tartrate, which is equivalent to about 117 μg of varenicline free base. Amounts of salt and the corresponding amount of free base may be calculated for other concentrations or volumes in a similar fashion.

The corresponding amount of a salt form may be calculated by multiplying the amount of free base by a multiplication factor. The multiplication factor is calculated by dividing the molecular weight of the salt form by the molecular weight of the free base. For instance, the multiplication factor for converting an amount of the compound of formula I free base to the mono-citrate salt is 2.096. The multiplication factor for converting an amount of the compound of formula I free base to the hemi-galactarate dihydrate is 1.805. The multiplication factor for converting an amount of varenicline free base to varenicline tartrate is 1.710.

The corresponding amount of the free base may be calculated by multiplying the amount of a salt form by a multiplication factor. The factor is calculated by dividing the molecular weight of the free base by the molecular weight of the salt form. For instance, the multiplication factor for converting an amount of varenicline tartrate to free base is 0.5846. The multiplication factor for converting an amount of the compound of formula I mono-citrate to free base is 0.477. The multiplication factor for converting an amount of the compound of formula I hemi-galactarate dihydrate to free base is 0.554.

In some of the embodiments disclosed herein, the nAChR agonist, or a pharmaceutically acceptable salt thereof, or the PAM, or pharmaceutically acceptable salt thereof, is administered to the nasal cavity of a subject. The pharmaceutical formulations described herein include, but are not limited to, liquids, suspensions, aerosols, gels, ointments, dry powders, creams, pastes, lotions, or balms.

In some of the embodiments disclosed herein, the nAChR agonist, or a pharmaceutically acceptable salt thereof, or the PAM, or pharmaceutically acceptable salt thereof, administered into the nasal cavity by a spray pump, syringe, dropper, bottle nebulizer, atomization pump, inhaler, powder spray device, vaporizer, patch, medicated stick, pipette, or jet of liquid.

In some of the embodiments disclosed herein, the PAM, or pharmaceutically acceptable salt thereof, is administered to the nasal cavity of the subject. In some of the embodiments disclosed herein, the PAM, or pharmaceutically acceptable salt thereof, is administered orally.

### Side Effects

The disclosure provides methods of local administration (intranasal) of the nAChR agonist, or pharmaceutically acceptable salts thereof, or the PAM, or pharmaceutically acceptable salts thereof. Local administration has the advantage over systemic administration including reducing potential side effects by limiting the amount of drug that may cross the blood-brain barrier. In some of the embodiments disclosed herein, the nAChR agonist, or pharmaceutically acceptable salt thereof, or the PAM, or a pharmaceutically acceptable salt thereof, administered is not systemically bioavailable. In some of the embodiments disclosed herein, the method does not result in undesired systemic side effects. In some of the embodiments disclosed herein, the method does not result in undesired psychoactive side effects.

In some of the embodiments described herein, the individual does not experience one or more side effects selected from the group consisting of overproduction of tears, cough, throat irritation, instillation site irritation, sneezing, nasopharyngitis, nasal irritation, toothache, dry mouth, and headache.

In some of the embodiments described herein, wherein within 5 minutes-60 minutes of administration of the first dose or one or more subsequent doses, the individual does not experience one or more side effects selected from the group consisting of overproduction of tears, cough, throat irritation, instillation site irritation, sneezing, nasopharyngitis, nasal irritation, toothache, dry mouth, and headache.

In some of the embodiments described herein, the overproduction of tears is indicated by an increase in an individual's Schirmer's score of greater than 20 mm. In some of the embodiments described herein, the overproduction of tears is indicated by excessive tearing, in an amount that would be impractical or undesirable. For example, tearing in an amount that would interfere with an individual's eye make-up, or would lead an individual to wipe away or absorb excess tears with a tissue, are both impractical and undesirable effects.

### Refractive Surgeries

In some of the embodiments described herein the individual will undergo or has undergone refractive surgery of the eye. Examples of refractive surgery include Laser-Assisted In-Situ Keratomileusis (LASIK), astigmatic keratotomy (AK), photorefractive keratectomy (PRK), and limbal relaxing incision (LRI). In some embodiments of the methods, uses and compositions for use, the refractive surgery is Laser-Assisted In-Situ Keratomileusis (LASIK). In some embodiments of the methods, uses and compositions for use, the refractive surgery is astigmatic keratotomy (AK). In some embodiments of the methods, uses and compositions for use, the refractive surgery is photorefractive keratectomy (PRK). In some embodiments of the methods, uses and compositions for use, the refractive surgery is limbal relaxing incision (LRI). In some embodiments, the some embodiments, the compounds or compositions described herein are administered prior to surgery. In some embodiments, the compounds or compositions described herein are administered prior to surgery. In some embodiments, the compounds or compositions described herein are administered both prior to and after surgery. In some of the embodiments described herein, the individual has undergone refractive surgery of the eye within 2 weeks or is scheduled to undergo refractive surgery of the eye within 2 weeks.

### Lasik Population

In some embodiments, the some embodiments, the compounds or compositions described herein are administered prior to surgery. In some embodiments, the compounds or compositions described herein are administered prior to surgery. In some embodiments, the compounds or compositions described herein are administered both prior to and after surgery. In some of the embodiments described herein, the individual has undergone Lasik surgery within 2 weeks or is scheduled to undergo Lasik surgery within 2 weeks.

### Keratitis

Normal tear film contains a number of biologically active growth factors including nerve growth factor, epidermal growth factor, transforming growth factor-beta, hepatocyte growth factor, platelet-derived growth factor, vascular endothelial growth factor, fibroblast growth factor, keratinocyte growth factor, and insulin-like growth factor. When the cornea senses stimulation or pressure, the eyelids will close and tears will be produced to protect the cornea and the eye.

Neurotrophic keratitis causes reduced sensitivity of the cornea. Subjects with neurotrophic keratitis (NK) have impaired cornea function because the nerves that innervate the cornea cannot function properly; these nerves carry impulses that help the cornea function. Because these nerves do not function properly in NK, the outer layer of the cornea, called the epithelium, can break down, resulting in an epithelial defect. In more advanced neurotrophic keratitis, an interior layer called the cornea stroma can break down as well, resulting in thinning of the cornea. This is called stromal ‘melting’. In advanced stromal melting, the cornea can thin to a severe degree, which can result in a hole or opening to the inside of the eye, which can lead to infection and potentially loss of the eye. NK can lead to a variety of complications, including poor wound healing of the cornea, scarring of the cornea, and loss of vision. There are many different conditions that can damage the nerves serving the cornea.

A variety of therapies can be used to treat this disorder depending on how far the disorder has progressed in an individual. Most recently, recombinant human nerve growth factor (rhNGF) (Oxervate, Dompe) has been approved for the treatment of NK. Unfortunately, there are several drawbacks to this therapy including the fact that the product needs to be administered 6 times per day at 2-hour intervals, for 8 weeks, the product is delivered with a pipette that can be cumbersome for self-administration, the product has to be refrigerated, and the cost for a 4-week supply is more than $10K.

The present disclosure provides methods, uses, and compositions for treating subjects with keratitis. In some embodiments, the keratitis is NK. In some embodiments, the methods and compositions for treating subject with NK disclosed herein stimulate natural tear film. In some embodiments, a subject with NK is administered a composition provided herein once or twice daily. In some embodiments, a subject with NK is administered a composition provided herein once or twice daily for at least 4, at least 5, at least 6, at least 7, or at least 8 weeks. In some embodiments, a subject with NK is administered a composition provided herein twice daily for at least 8 weeks. In some embodiments, a subject with NK is intranasally administered a composition provided herein twice daily for at least 8 weeks.

### Cmax Blood Plasma Concentration

The methods and uses described herein include local intranasal administration. Because the disclosed methods and uses are to local intranasal administration, the concentration of the nAChR agonist, or pharmaceutically acceptable salt thereof, in the circulating blood plasma is low, compared to concentrations achieved from systemic forms of administration (e.g. ingestion of oral formulations). Low blood plasma concentrations of nAChR agonists avoids potential undesirable side effects associated with nAChR agonists in systemic circulation, such as nausea, sleep disturbance, constipation, flatulence, vomiting, dermal conditions like rash and pruritus, headaches, abdominal pain, dyspepsia, gastroesophageal reflux disease and dry mouth.

One way to characterize the blood plasma concentration of the nAChR agonist, or pharmaceutically acceptable salt thereof, in an individual is to measure the Cmax—the maximum or peak serum concentration that the nAChR agonist, or pharmaceutically acceptable salt thereof, achieves after the nAChR agonist, or pharmaceutically acceptable salt thereof, is administered to the individual and before the administration of a second dose. Methods of determining Cmax are known in the art. A non-limiting example of a protocol used to determine the pharmacokinetic profile of the compound of formula I, including determining Cmax is provided in Example 3.

In some embodiments described herein, the Cmax is calculated for an individual. In some embodiments, Cmax is calculated from the average Cmax of two or more individuals. In some embodiments, Cmax is calculated from the average Cmax of a subset population.

In some embodiments described herein, the individual in need thereof has a blood plasma Cmax of the nAChR agonist, or a pharmaceutically acceptable salt thereof, of less than 5 ng/mL, of less than 4 ng/mL, of less than 3 ng/mL, or of less than 2 ng/mL.

In some embodiments described herein, the individual in need thereof has a blood plasma Cmax of the nAChR agonist, or a pharmaceutically acceptable salt thereof, of less than 5 ng/mL, of less than 4 ng/mL, of less than 3 ng/mL, or of less than 2 ng/mL; and wherein the individual in need thereof was administered a dose comprising between 10 micrograms to 150 micrograms, 10 micrograms to 100 micrograms, 10 micrograms to 50 micrograms, 50 micrograms to 150 micrograms, 50 micrograms to 100 micrograms, 100 micrograms to 1500 micrograms, 100 micrograms to 600 micrograms, 200 micrograms to 400 micrograms, 400 micrograms to 600 micrograms, or 750 micrograms to 1200 micrograms of the nAChR agonist, or a corresponding amount of the pharmaceutically acceptable salt thereof.

In some embodiments described herein, the individual in need thereof has a blood plasma Cmax of compound 1, or a pharmaceutically acceptable salt thereof, of less than 5 ng/mL, of less than 4 ng/mL, of less than 3 ng/mL, or of less than 2 ng/mL; and wherein the individual in need thereof was administered a dose comprising between 100 micrograms to 1500 micrograms, 100 micrograms to 600 micrograms, 200 micrograms to 400 micrograms, 400 micrograms to 600 micrograms, or 750 micrograms to 1200 micrograms of compound 1, or a corresponding amount of the pharmaceutically acceptable salt thereof.

In some embodiments described herein, the individual in need thereof has a blood plasma Cmax of compound 1, or a pharmaceutically acceptable salt thereof, of less than 5 ng/mL, of less than 4 ng/mL, of less than 3 ng/mL, or of less than 2 ng/mL; and wherein the individual in need thereof was administered a dose comprising between 150 micrograms to 300 micrograms, 900 micrograms to 1200 micrograms, 2100 micrograms to 2400 micrograms of compound 1, or a corresponding amount of the pharmaceutically acceptable salt thereof.

In some embodiments described herein, the individual in need thereof has a blood plasma Cmax of varenicline, or a pharmaceutically acceptable salt thereof, of less than 5 ng/mL, of less than 4 ng/mL, of less than 3 ng/mL, or of less than 2 ng/mL; and wherein the individual in need thereof was administered a dose comprising between 5 micrograms to 15 micrograms, 50 micrograms to 65 micrograms, 100 micrograms to 125 micrograms of varenicline, or a corresponding amount of the pharmaceutically acceptable salt thereof.

In some of the embodiments described herein, effective treatment of the individual is indicated by an Eye Dryness Score test on a visual analog scale and/or a Schirmer's test, the statistically significant decrease in the individual's Eye Dryness Score is within 5 minutes of administration of the first dose of an effective amount of compound 1, or a pharmaceutically acceptable salt thereof, and is characterized by a p value of less than 0.05. In some of the embodiments described herein, the statistically significant decrease in the individual's Eye Dryness score is at least 10 mm. In some of the embodiments described herein, the statistically significant decrease in the individual's Eye Dryness score is at least 5 mm. In some of the embodiments described herein, the statistically significant decrease in the individual's Eye Dryness score is at least 3 mm. In yet further embodiments of any of the embodiments described herein, 1800-2500 micrograms of compound 1, or a corresponding amount of a pharmaceutically acceptable salt thereof, per dose is administered to the individual. In yet further embodiments of any of the embodiments described herein, 3500-4500 micrograms of compound 1, or a corresponding amount of a pharmaceutically acceptable salt thereof, per dose is administered to the individual. In some of the embodiments described herein, compound 1, or a pharmaceutically acceptable salt thereof, is administered in a pharmaceutical formulation for nasal administration, and the total volume of the pharmaceutical formulation per dose is 50 microliters-250 microliters. In some of the embodiments described herein, the total volume of the pharmaceutical formulation per dose is 75 microliters-150 microliters. In some of the embodiments described herein, the total volume of the pharmaceutical formulation per dose is 150 microliters-250 microliters. In some of the embodiments described herein, the total volume of the pharmaceutical formulation per dose is about 100 microliters or about 200 microliters. In some of the embodiments described herein, the pharmaceutical formulation for nasal administration comprises between 1 mg/mL and 40 mg/mL of compound 1, or a corresponding amount of a pharmaceutically acceptable salt thereof. In some of the embodiments described herein, the pharmaceutical formulation for nasal administration comprises between 5 mg/mL and 25 mg/mL of compound 1, or a corresponding amount of a pharmaceutically acceptable salt thereof. In some of the embodiments described herein, the pharmaceutical formulation for nasal administration comprises 2 mg/mL, 10 mg/mL, or 20 mg/mL of compound 1, or a corresponding amount of a pharmaceutically acceptable salt thereof. In some of the embodiments described herein, the statistically significant decrease in the individual's Eye Dryness Score is maintained within 10% or 20% for at least 30 minutes from administration of the first dose of an effective amount of compound 1, or a pharmaceutically acceptable salt thereof. In some of the embodiments described herein, the statistically significant decrease in the individual's Eye Dryness Score is maintained within 10% or 20% for at least 60 minutes from administration of the first dose of an effective amount of compound 1, or a pharmaceutically acceptable salt thereof. In some of the embodiments described herein, the individual's Eye Dryness Score 30 minutes after administering the first dose of an effective amount of compound 1, or pharmaceutically acceptable salt thereof, is within 10% of the individual's Eye Dryness Score within 5 minutes after administering the first dose of an effective amount of compound 1, or pharmaceutically acceptable salt thereof.

In some of the embodiments described herein, the method comprises administering into a nasal cavity of the individual in need thereof, a first dose, and optionally one or more subsequent doses, of an effective amount of compound 1, or a pharmaceutically acceptable salt thereof, wherein the method results in the effective treatment of the individual in need thereof. In some of the embodiments described herein, effective treatment of the individual is indicated by one or more of the tests selected from the group consisting of a) Schirmer's score Test on a visual analog scale and b) Schirmer's test. In some of the embodiments described herein, the individual's Schirmer's score is compared to a Schirmer's score of the individual prior to administration of the first dose of compound 1, or a pharmaceutically acceptable salt thereof the statistically significant increase in the individual's Schirmer's score is within 5 minutes of administration of the first dose of an effective amount of compound 1, or a pharmaceutically acceptable salt thereof, and is characterized by a p value of less than 0.05. In some of the embodiments described herein, the statistically significant increase in the individual's Schirmer's score is at least 100%, 200% or 300%. In some of the embodiments described herein, the statistically significant increase in the individual's Schirmer's score is at least 10 mm. In some of the embodiments described herein, the statistically significant increase in the individual's Schirmer's score is at least 5 mm. In some of the embodiments described herein, the statistically significant increase in the individual's Schirmer's score is at least 3 mm. In yet further embodiments of any of the embodiments described herein, 1800-2500 micrograms of the pharmaceutically acceptable salt of compound 1 per dose is administered to the individual. In yet further embodiments of any of the embodiments described herein, 3500-4500 micrograms of the pharmaceutically acceptable salt of compound 1 per dose is administered to the individual. In some of the embodiments described herein, compound 1, or a pharmaceutically acceptable salt thereof, is administered in a pharmaceutical formulation for nasal administration, and the total volume of the pharmaceutical formulation per dose is 50 microliters-250 microliters. In some of the embodiments described herein, the total volume of the pharmaceutical formulation per dose is 75 microliters-150 microliters. In some of the embodiments described herein, the total volume of the pharmaceutical formulation per dose is 150 microliters-250 microliters. In some of the embodiments described herein, the total volume of the pharmaceutical formulation per dose is about 100 microliters or about 200 microliters. In some of the embodiments described herein, the pharmaceutical formulation for nasal administration comprises between 1 mg/mL and 40 mg/mL of compound 1, or a corresponding amount of a pharmaceutically acceptable salt thereof. In some of the embodiments described herein, the pharmaceutical formulation for nasal administration comprises between 5 mg/mL and 25 mg/mL of compound 1, or a corresponding amount of a pharmaceutically acceptable salt thereof. In some of the embodiments described herein, the pharmaceutical formulation for nasal administration comprises about 2 mg/mL, 10 mg/mL, or 20 mg/mL of compound 1, or a corresponding amount of a pharmaceutically acceptable salt thereof. In some of the embodiments described herein, the statistically significant increase in the individual's Schirmer's score is maintained within 10% or 20% for at least 30 minutes from administration of the first dose of an effective amount of compound 1, or a pharmaceutically acceptable salt thereof. In some of the embodiments described herein, the statistically significant increase in the individual's Schirmer's score is maintained within 10% or 20% for at least 60 minutes from administration of the first dose of an effective amount of compound 1, or a pharmaceutically acceptable salt thereof. In some of the embodiments described herein, the individual's Schirmer's score 30 minutes after administering the first dose of an effective amount of compound 1, or pharmaceutically acceptable salt thereof, is within 10% of the individual's Schirmer's score within 5 minutes after administering the first dose of an effective amount of compound 1, or pharmaceutically acceptable salt thereof.

Comparator compounds, as used herein, include but are not limited to Restasis® and Xiidra®.

A statistically significant change, as used herein, to describe an individual's score may be calculated from an increase or decrease in the individual's score. Non-limiting examples of determining whether the change in an individual's score is statistically significant and whether two sets of data are significantly different from each other include calculations based on an ANCOVA model, and statistical hypothesis tests such as t-tests and non-parametric Wilcoxon rank sum tests. Other models and statistical hypothesis tests well-known in the art are contemplated.

Throughout the present disclosure, amounts of nAChR agonists or PAM disclosed refer to the amount of nAChR agonist free form or PAM free form present in the formulation. The term “corresponding amount” as used herein refers to the amount of a pharmaceutically acceptable salt of a nAChR agonist or a PAM required to obtain the amount of nAChR free form or PAM free form recited in the formulation. It would be clear to one of skill in the art how to calculate the “corresponding amount” of the salt of a compound, such as the corresponding amount of the pharmaceutically acceptable salt of compound 1, taking into account the difference in molecular weight between the free form of a compound and a salt form. For example, 175.24 g of compound 1 free base, would correspond to 316.34 g of the hemi-galactarate dihydrate salt or 367.36 of the mono-citrate salt. In another example, 211.267 g of varenicline free base, would correspond to 361.354 of the varenicline tartrate salt.

As used herein, the term “about” is used synonymously with the term “approximately.” Illustratively, the use of the term “about” with regard to an amount indicates values slightly outside the cited values, e.g., plus or minus 0.1% to 20%, plus or minus 0.1% to 10%, plus or minus 0.1% to 5%, or plus or minus 0.1% to 2%.

Throughout the present disclosure, various embodiments discussed within the context of the methods of treating dry eye disease, increasing tear production, and reducing ocular discomfort may also be applied to the embodiments related to compounds for use in the manufacture of a medicament, compounds for use in the various methods, pharmaceutical formulations, and kits.

### Exemplary Embodiments

Embodiment I-1. A method of treating dry eye disease, increasing tear production, or reducing ocular discomfort in an individual in need thereof, comprising administering a first dose, and optionally one or more subsequent doses, of an effective amount of a nicotinic acetylcholine receptor (nAChR) agonist, or a pharmaceutically acceptable salt thereof, and a first dose, and optionally one or more subsequent doses, of an effective amount of a positive allosteric modulator (PAM), or a pharmaceutically acceptable salt thereof, into a nasal cavity of the individual in need thereof, wherein the nAChR agonist, or a pharmaceutically acceptable salt thereof, is varenicline, or a pharmaceutically acceptable salt thereof, or compound 1 having the structure

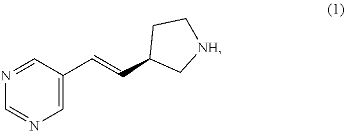 [SMILES: C(=C\[C@H]1CCNC1)/c1cncnc1]

or a pharmaceutically acceptable salt thereof, wherein the method results in the effective treatment of the individual in need thereof.

Embodiment I-2. A compound for use in a method of treating dry eye disease, increasing tear production, or reducing ocular discomfort in an individual in need thereof, wherein the compound is a nAChR agonist, or a pharmaceutically acceptable salt thereof, wherein the method comprises administering a first dose, and optionally one or more subsequent doses, of an effective amount of a nAChR agonist, or a pharmaceutically acceptable salt thereof, a first dose, and optionally one or more subsequent doses, of an effective amount of a PAM, or a pharmaceutically acceptable salt thereof, into a nasal cavity of the individual in need thereof, wherein the nAChR agonist, or a pharmaceutically acceptable salt thereof, is varenicline, or a pharmaceutically acceptable salt thereof, or compound 1 having the structure

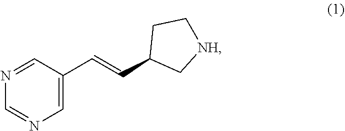 [SMILES: C(=C\[C@H]1CCNC1)/c1cncnc1]

or a pharmaceutically acceptable salt thereof.

Embodiment I-3. A compound for use in a method of treating dry eye disease, increasing tear production, or reducing ocular discomfort in an individual in need thereof, wherein the compound is a PAM, or a pharmaceutically acceptable salt thereof, wherein the method comprises administering a first dose, and optionally one or more subsequent doses, of an effective amount of a nAChR agonist, or a pharmaceutically acceptable salt thereof, and a first dose, and optionally one or more subsequent doses, of an effective amount of a PAM, or a pharmaceutically acceptable salt thereof, into a nasal cavity of the individual in need thereof, and wherein the nAChR agonist, or a pharmaceutically acceptable salt thereof, is varenicline, or a pharmaceutically acceptable salt thereof, or compound 1 having the structure

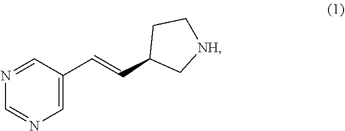 [SMILES: C(=C\[C@H]1CCNC1)/c1cncnc1]

or a pharmaceutically acceptable salt thereof.

Embodiment I-4. A combined preparation of: (i) a nAChR agonist, or a pharmaceutically acceptable salt thereof, and (ii) a PAM, or a pharmaceutically acceptable salt thereof, for simultaneous, separate or sequential use in a method of treating dry eye disease, increasing tear production, or reducing ocular discomfort in an individual in need thereof, wherein the nAChR agonist is varenicline, compound 1 having the structure

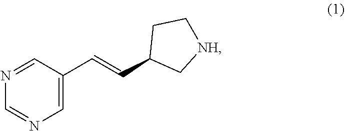 [SMILES: C(=C\[C@H]1CCNC1)/c1cncnc1]

or a pharmaceutically acceptable salt thereof, and wherein the method comprises administering a first dose, and optionally one or more subsequent doses, of an effective amount of the nAChR agonist, or pharmaceutically acceptable salt thereof, and a first dose, and optionally one or more subsequent doses, of an effective amount of the PAM, or pharmaceutically acceptable salt thereof, into a nasal cavity of the individual in need thereof.

Embodiment I-5. The compound for use of Embodiment I-2 or 1-3 or the combined preparation of Embodiment I-4 wherein the method results in the effective treatment of the individual in need thereof.

Embodiment I-6. The method of Embodiment I-1, the compound for use of Embodiment I-5, or the combined preparation of Embodiment I-5, wherein the effective treatment of the individual is indicated by one or more of the tests selected from the group consisting of a) Eye Dryness score test on a visual analog scale, b) Schirmer's test, c) Corneal Fluorescein Staining test, and d) Ocular Surface Disease Index test.

Embodiment I-7. The method, compound for use, or combined preparation of Embodiment I-6, wherein the effective treatment is indicated by a statistically significant decrease in the individual's Eye Dryness score, and wherein the statistically significant decrease in the individual's Eye Dryness score is determined after administration to the individual of the first dose, or the optionally one or more subsequent doses, of the nAChR agonist, or a pharmaceutically acceptable salt thereof, and the first dose, or the optionally one or more subsequent doses, of the PAM, or a pharmaceutically acceptable salt thereof, wherein the individual's Eye Dryness score is compared to a) an Eye Dryness score of the individual prior to administration of the first dose of the nAChR agonist, or a pharmaceutically acceptable salt thereof, and the first dose of the PAM, or a pharmaceutically acceptable salt thereof; b) an Eye Dryness score of an individual administered a control; or c) an Eye Dryness score of an individual administered a comparator compound.

Embodiment I-8. The method, compound for use, or combined preparation of Embodiment I-7, wherein the statistically significant decrease in the individual's Eye Dryness score is at least 15%.

Embodiment I-9. The method, compound for use, or combined preparation of Embodiment I-7 or Embodiment I-8, wherein the statistically significant decrease in the individual's Eye Dryness score is between 10 mm and 20 mm.

Embodiment I-10. The method, compound for use, or combined preparation of any one of Embodiments I-7 to I-9, wherein the statistically significant decrease in the individual's Eye Dryness score is at least 10 mm,

Embodiment I-11. The method, compound for use, or combined preparation of any one of Embodiments I-7 to I-10, wherein the statistically significant decrease in the individual's Eye Dryness score is characterized by a p value of 0.05 or less, 0.01 or less, 0.005 or less, or 0.001 or less.

Embodiment I-12. The method, compound for use, or combined preparation of any one of Embodiments I-7 to I-11, wherein the statistically significant decrease in the individual's Eye Dryness score is within 5 minutes of administration of the first dose of the nAChR agonist, or a pharmaceutically acceptable salt thereof, and, optionally, the first dose of the PAM, or a pharmaceutically acceptable salt thereof.

Embodiment I-13. The method, compound for use, or combined preparation of any one of Embodiments I-7 to I-12, wherein the statistically significant improvement in the individual's Eye Dryness score is maintained for at least 30 minutes from administration of the first dose of the nAChR agonist, or a pharmaceutically acceptable salt thereof, and, optionally, the first dose of the PAM, or a pharmaceutically acceptable salt thereof.

Embodiment I-14. The method, compound for use, or combined preparation of any one of Embodiments I-7 to I-13, wherein the individual's Eye Dryness score at least 30 minutes after administering the first dose of the nAChR agonist, or a pharmaceutically acceptable salt thereof, and, optionally, the first dose of the PAM, or a pharmaceutically acceptable salt thereof, does not increase by more than 30% of the individual's Eye Dryness score within 5 minutes after administering the first dose of the nAChR agonist, or a pharmaceutically acceptable salt thereof, and, optionally, the first dose of the PAM, or a pharmaceutically acceptable salt thereof.

Embodiment I-15. The method, compound for use, or combined preparation of any one of Embodiments I-7 to I-14, wherein, between determinations of the individual's Eye Dryness score, the individual is present in an environment with reduced humidity.

Embodiment I-16. The method, compound for use, or combined preparation of any one of Embodiments I-7 to I-15, comprises administering a first dose, and one or more subsequent doses, of an effective amount of a PAM, or a pharmaceutically acceptable salt thereof, and a first dose, and one or more subsequent doses, of an effective amount of a nAChR agonist, or a pharmaceutically acceptable salt thereof.

Embodiment I-17. The method, compound for use, or combined preparation of any one of Embodiments I-7 to I-16, wherein the statistically significant decrease in the individual's Eye Dryness score is based on the individual's Eye Dryness score determined after administering the first dose of the nAChR agonist, or a pharmaceutically acceptable salt thereof.

Embodiment I-18. The method, compound for use, or combined preparation of any one of Embodiments I-7 to I-16, wherein the statistically significant decrease is based on the individual's Eye Dryness score determined after administering one or more subsequent doses of the nAChR agonist, or a pharmaceutically acceptable salt thereof, and optionally the first or the one or more subsequent doses of the PAM, or a pharmaceutically acceptable salt thereof.

Embodiment I-19. The method, compound for use, or combined preparation of any one of Embodiments I-6 to I-18, wherein the effective treatment is indicated by a statistically significant increase in the individual's Schirmer's score, and wherein the statistically significant increase in the individual's Schirmer's score in at least one eye is determined after administration to the individual of the first dose, or the optionally one or more subsequent doses, of the nAChR agonist, or a pharmaceutically acceptable salt thereof, and the first dose, or the optionally one or more subsequent doses, of the PAM, or a pharmaceutically acceptable salt thereof, wherein the individual's Schirmer's score is compared to a) a Schirmer's score of the individual prior to administration of the first dose of the nAChR agonist, or a pharmaceutically acceptable salt thereof, and the first dose of the PAM, or a pharmaceutically acceptable salt thereof; b) a Schirmer's score of an individual administered a control; or c) a Schirmer's score of an individual administered a comparator compound.

Embodiment I-20. The method, compound for use, or combined preparation of Embodiment I-19, wherein the statistically significant increase in the individual's Schirmer's score is at least 15%.

Embodiment I-21. The method, compound for use, or combined preparation of Embodiment I-19 or 1-20, wherein the statistically significant increase in the individual's Schirmer's score is between 10 mm and 20 mm.

Embodiment I-22. The method, compound for use, or combined preparation of any one of Embodiments I-19 to I-21, wherein the statistically significant increase in the individual's Schirmer's score is at least 10 mm.

Embodiment I-23. The method, compound for use, or combined preparation of any one of Embodiments I-19 to I-22, wherein the statistically significant increase in the individual's Schirmer's score is characterized by a p value of 0.05 or less, 0.01 or less, 0.005 or less, or 0.001 or less.

Embodiment I-24. The method, compound for use, or combined preparation of any one of Embodiments I-19 to I-23, wherein the statistically significant increase in the individual's Schirmer's score is within 5 minutes of administration of the first dose of the nAChR agonist, or a pharmaceutically acceptable salt thereof, and, optionally, the first dose of the PAM, or a pharmaceutically acceptable salt thereof.

Embodiment I-25. The method, compound for use, or combined preparation of any one of Embodiments I-19 to I-24, wherein the statistically significant improvement in the individual's Schirmer's score is maintained for at least 30 minutes from administration of the first dose of the nAChR agonist, or a pharmaceutically acceptable salt thereof, and, optionally, the first dose of the PAM, or a pharmaceutically acceptable salt thereof.

Embodiment I-26. The method, compound for use, or combined preparation of any one of Embodiments I-19 to I-25, wherein the individual's Schirmer's score at least 30 minutes after administering the first dose of the nAChR agonist, or a pharmaceutically acceptable salt thereof, and, optionally, the first dose of the PAM, or a pharmaceutically acceptable salt thereof, does not decrease by more than 30% of the individual's Schirmer's score within 5 minutes after administering the first dose of the nAChR agonist, or a pharmaceutically acceptable salt thereof, and, optionally, the first dose of the PAM, or a pharmaceutically acceptable salt thereof.

Embodiment I-27. The method, compound for use, or combined preparation of any one of Embodiments I-19 to I-26, wherein, between determinations of the individual's Schirmer's score, the individual is present in an environment with reduced humidity.

Embodiment I-28. The method, compound for use, or combined preparation of any one of Embodiments I-19 to I-27, comprises administering a first dose, and one or more subsequent doses, of an effective amount of a PAM, or a pharmaceutically acceptable salt thereof,

and a first dose, and one or more subsequent doses, of an effective amount of a nAChR agonist, or a pharmaceutically acceptable salt thereof.

Embodiment I-29. The method, compound for use, or combined preparation of any one of Embodiments I-19 to I-28, wherein the statistically significant increase in the individual's Schirmer's score is based on the individual's Schirmer's score determined after administering the first dose of the nAChR agonist, or a pharmaceutically acceptable salt thereof.

Embodiment I-30. The method, compound for use, or combined preparation of any one of Embodiments I-19 to I-28, wherein the statistically significant increase is based on the individual's Schirmer's score determined after administering one or more subsequent doses of the nAChR agonist, or a pharmaceutically acceptable salt thereof, and optionally the first or the one or more subsequent doses of the PAM, or a pharmaceutically acceptable salt thereof.

Embodiment I-31. The method, compound for use, or combined preparation of any one of Embodiments I-6 to I-30, wherein the effective treatment is indicated by a statistically significant decrease in the individual's corneal score, and wherein the statistically significant decrease in the individual's corneal score is determined after administration to the individual of the first dose, or the optionally one or more subsequent doses, of the nAChR agonist, or a pharmaceutically acceptable salt thereof, and the first dose, or the optionally one or more subsequent doses, of the PAM, or a pharmaceutically acceptable salt thereof, wherein the individual's corneal score is compared to

a) a corneal score of the individual prior to administration of the first dose of the nAChR agonist, or a pharmaceutically acceptable salt thereof, and the first dose of the PAM, or a pharmaceutically acceptable salt thereof; b) a corneal score of an individual administered a control; or c) a corneal score of an individual administered a comparator compound.

Embodiment I-32. The method, compound for use, or combined preparation of Embodiment I-31, wherein the statistically significant decrease in the individual's corneal score is at least 15%.

Embodiment I-33. The method, compound for use, or combined preparation of Embodiment I-31 or 1-32, wherein the statistically significant decrease in the individual's corneal score is characterized by a p value of 0.05 or less, 0.01 or less, 0.005 or less, or 0.001 or less.

Embodiment I-34. The method, compound for use, or combined preparation of any one of Embodiments I-31 to I-33, wherein the statistically significant decrease in the individual's corneal score is within 5 minutes of administration of the first dose of the nAChR agonist, or a pharmaceutically acceptable salt thereof, and, optionally, the first dose of the PAM, or a pharmaceutically acceptable salt thereof.

Embodiment I-35. The method, compound for use, or combined preparation of any one of Embodiments I-31 to I-34, wherein the statistically significant improvement in the individual's corneal score is maintained for at least 30 minutes from administration of the first dose of the nAChR agonist, or a pharmaceutically acceptable salt thereof, and, optionally, the first dose of the PAM, or a pharmaceutically acceptable salt thereof.

Embodiment I-36. The method, compound for use, or combined preparation of any one of Embodiments I-31 to I-35, wherein the individual's corneal score at least 30 minutes after administering the first dose of the nAChR agonist, or a pharmaceutically acceptable salt thereof, and, optionally, the first dose of the PAM, or a pharmaceutically acceptable salt thereof, does not increase by more than 30% of the individual's corneal score within 5 minutes after administering the first dose of the nAChR agonist, or a pharmaceutically acceptable salt thereof, and, optionally, the first dose of the PAM, or a pharmaceutically acceptable salt thereof.

Embodiment I-37. The method, compound for use, or combined preparation of any one of Embodiments I-31 to I-36, wherein, between determinations of the individual's corneal score, the individual is present in an environment with reduced humidity.

Embodiment I-38. The method, compound for use, or combined preparation of any one of Embodiments I-31 to I-37, comprises administering a first dose, and one or more subsequent doses, of an effective amount of a PAM, or a pharmaceutically acceptable salt thereof, and a first dose, and one or more subsequent doses, of an effective amount of a nAChR agonist, or a pharmaceutically acceptable salt thereof.

Embodiment I-39. The method, compound for use, or combined preparation of any one of Embodiments I-31 to I-38, wherein the statistically significant decrease in the individual's corneal score is based on the individual's corneal score determined after administering the first dose of the nAChR agonist, or a pharmaceutically acceptable salt thereof.

Embodiment I-40. The method, compound for use, or combined preparation of any one of Embodiments I-31 to I-38, wherein the statistically significant decrease is based on the individual's corneal score determined after administering one or more subsequent doses of the nAChR agonist, or a pharmaceutically acceptable salt thereof, and optionally the first or the one or more subsequent doses of the PAM, or a pharmaceutically acceptable salt thereof.

Embodiment I-41. The method, compound for use, or combined preparation of any one of Embodiments I-6 to I-40, wherein the effective treatment is indicated by a statistically significant decrease in the individual's OSDI score, and wherein the statistically significant decrease in the individual's OSDI score is determined after administration to the individual of the first dose, or the optionally one or more subsequent doses, of the nAChR agonist, or a pharmaceutically acceptable salt thereof, and the first dose, or the optionally one or more subsequent doses, of the PAM, or a pharmaceutically acceptable salt thereof, wherein the individual's OSDI score is compared to

a) a OSDI score of the individual prior to administration of the first dose of the nAChR agonist, or a pharmaceutically acceptable salt thereof, and the first dose of the PAM, or a pharmaceutically acceptable salt thereof; b) a OSDI score of an individual administered a control; or c) a OSDI score of an individual administered a comparator compound.

Embodiment I-42. The method, compound for use, or combined preparation of Embodiment I-41, wherein the statistically significant decrease in the individual's OSDI score is at least 15%.

Embodiment I-43. The method, compound for use, or combined preparation of Embodiment I-41 or 1-42, wherein the statistically significant decrease in the individual's OSDI score is characterized by a p value of 0.05 or less, 0.01 or less, 0.005 or less, or 0.001 or less.

Embodiment I-44. The method, compound for use, or combined preparation of any one of Embodiments I-41 to I-43, wherein the statistically significant decrease in the individual's OSDI score is within 5 minutes of administration of the first dose of the nAChR agonist, or a pharmaceutically acceptable salt thereof, and, optionally, the first dose of the PAM, or a pharmaceutically acceptable salt thereof.

Embodiment I-45. The method, compound for use, or combined preparation of any one of Embodiments I-41 to I-44, wherein the statistically significant improvement in the individual's OSDI score is maintained for at least 30 minutes from administration of the first dose of the nAChR agonist, or a pharmaceutically acceptable salt thereof, and, optionally, the first dose of the PAM, or a pharmaceutically acceptable salt thereof.

Embodiment I-46. The method, compound for use, or combined preparation of any one of Embodiments I-41 to I-45, wherein the individual's OSDI score at least 30 minutes after administering the first dose of the nAChR agonist, or a pharmaceutically acceptable salt thereof, and, optionally, the first dose of the PAM, or a pharmaceutically acceptable salt thereof, does not increase by more than 30% of the individual's OSDI score within 5 minutes after administering the first dose of the nAChR agonist, or a pharmaceutically acceptable salt thereof, and, optionally, the first dose of the PAM, or a pharmaceutically acceptable salt thereof.

Embodiment I-47. The method, compound for use, or combined preparation of any one of Embodiments I-41 to I-46, wherein, between determinations of the individual's OSDI score, the individual is present in an environment with reduced humidity.

Embodiment I-48. The method, compound for use, or combined preparation of any one of Embodiments I-41 to I-47, comprises administering a first dose, and one or more subsequent doses, of an effective amount of a PAM, or a pharmaceutically acceptable salt thereof, and a first dose, and one or more subsequent doses, of an effective amount of a nAChR agonist, or a pharmaceutically acceptable salt thereof.

Embodiment I-49. The method, compound for use, or combined preparation of any one of Embodiments I-41 to I-48, wherein the statistically significant decrease in the individual's OSDI score is based on the individual's OSDI score determined after administering the first dose of the nAChR agonist, or a pharmaceutically acceptable salt thereof.

Embodiment I-50. The method, compound for use, or combined preparation of any one of Embodiments I-41 to I-48, wherein the statistically significant decrease is based on the individual's OSDI score determined after administering one or more subsequent doses of the nAChR agonist, or a pharmaceutically acceptable salt thereof, and optionally the first or the one or more subsequent doses of the PAM, or a pharmaceutically acceptable salt thereof.

Embodiment I-51. The method, compound for use, or combined preparation of any one of Embodiments I-1 to I-50, wherein 5-4000 micrograms of the nAChR agonist, or a corresponding amount of a pharmaceutically acceptable salt thereof, per dose is administered to the individual.

Embodiment I-52. The method, compound for use, or combined preparation of any one of Embodiments I-1 to I-51, wherein the nAChR agonist, or a pharmaceutically acceptable salt thereof, is administered in a pharmaceutical formulation for nasal administration comprising between 1 mg/mL and 40 mg/mL of nAChR agonist, or a corresponding amount of a pharmaceutically acceptable salt thereof.

Embodiment I-53. The method, compound for use, or combined preparation of any one of Embodiments I-1 to I-52, wherein the first dose, and the optionally one or more subsequent doses of the nAChR agonist, or a pharmaceutically acceptable salt thereof, is administered in a pharmaceutical formulation for nasal administration, and the total volume of the pharmaceutical formulation administered per dose of the nAChR agonist, or a pharmaceutically acceptable salt thereof, to the individual is 50 microliters-250 microliters.

Embodiment I-54. The method, compound for use, or combined preparation of any one of Embodiments I-1 to I-53, wherein the first dose, and the optionally one or more subsequent doses, of the nAChR agonist, or a pharmaceutically acceptable salt thereof, is administered in a pharmaceutical formulation for nasal administration, and the total volume of the pharmaceutical formulation administered per nostril of the nAChR agonist, or a pharmaceutically acceptable salt thereof, to the individual is 50 microliters-250 microliters.

Embodiment I-55. The method, compound for use, or combined preparation of any one of Embodiments I-1 to I-54, wherein the first dose, and the optionally one or more subsequent doses, of the nAChR agonist, is a pharmaceutically acceptable salt thereof.

Embodiment I-56. The method, compound for use, or combined preparation of any one of Embodiments I-1 to I-55, wherein the PAM, or the pharmaceutically acceptable salt thereof, and the nAChR agonist, or the pharmaceutically acceptable salt thereof, are administered to the individual in need thereof in separate dosage forms.

Embodiment I-57. The method, compound for use, or combined preparation of any one of Embodiments I-1 to I-55, wherein the PAM, or the pharmaceutically acceptable salt thereof, and the nAChR agonist, or the pharmaceutically acceptable salt thereof, are administered to the individual in need thereof in a combined dosage form.

Embodiment I-58. The method, compound for use, or combined preparation of any one of Embodiments I-1 to I-55, wherein the PAM, or the pharmaceutically acceptable salt thereof, is administered to the individual in need thereof administered after the nAChR agonist, or the pharmaceutically acceptable salt thereof.

Embodiment I-59. The method, compound for use, or combined preparation of any one of Embodiments I-1 to I-56, wherein the PAM, or the pharmaceutically acceptable salt thereof, is administered to the individual in need thereof before the nAChR agonist, or the pharmaceutically acceptable salt thereof.

Embodiment I-60. The method, compound for use, or combined preparation of any one of Embodiments I-1 to I-57, wherein the PAM, or the pharmaceutically acceptable salt thereof, and the nAChR agonist, or the pharmaceutically acceptable salt thereof, are administered to the individual in need thereof at the same time.

Embodiment I-61. The method, compound for use, or combined preparation of any one of Embodiments I-1 to I-60, wherein a dose of the nAChR agonist, or the pharmaceutically acceptable salt thereof, and optionally a dose of the PAM, or a pharmaceutically acceptable salt thereof, is administered to the individual in need thereof one to four times daily after the first day of administration.

Embodiment I-62. The method, compound for use, or combined preparation of any one of Embodiments I-1 to I-61, wherein a) the dose frequency of the nAChR agonist, or the pharmaceutically acceptable salt thereof, orb) the total amount of the nAChR agonist, or the corresponding amount of the pharmaceutically acceptable salt thereof, per dose is reduced over time.

Embodiment I-63. The method, compound for use, or combined preparation of Embodiment I-62, wherein each dose of the nAChR agonist, or the pharmaceutically acceptable salt thereof, comprises more than one administration of the nAChR agonist, or the pharmaceutically acceptable salt thereof, into the nasal cavity of the individual, and wherein the reduction of the total amount of the nAChR agonist, or the corresponding amount of pharmaceutically acceptable salt thereof, per dose over time is accomplished by reducing the number of administrations of the nAChR agonist, or the pharmaceutically acceptable salt thereof, into the nasal cavity of the individual.

Embodiment I-64. The method, compound for use, or combined preparation of any one of Embodiments I-1 to I-63, wherein the dose of the nAChR agonist, or the pharmaceutically acceptable salt thereof, and optionally the PAM, or a pharmaceutically acceptable salt thereof, comprises more than one administration of the nAChR agonist, or pharmaceutically acceptable salt thereof, and optionally the PAM, or a pharmaceutically acceptable salt thereof, to one or both nostrils.

Embodiment I-65. The method, compound for use, or combined preparation of any one of Embodiments I-1 to I-63, wherein the dose of the nAChR agonist, or the pharmaceutically acceptable salt thereof, and optionally the PAM, or a pharmaceutically acceptable salt thereof, comprises a single administration of the nAChR agonist, or pharmaceutically acceptable salt thereof, and optionally the PAM, or a pharmaceutically acceptable salt thereof, to one or both nostrils.

Embodiment I-66. The method, compound for use, or combined preparation of any one of Embodiments I-1 to I-65, wherein the nAChR agonist, or the pharmaceutically acceptable salt thereof, and optionally the PAM, or a pharmaceutically acceptable salt thereof, is administered to one nostril per dose.

Embodiment I-67. The method, compound for use, or combined preparation of any one of Embodiments I-1 to I-65, wherein the nAChR agonist, or the pharmaceutically acceptable salt thereof, and optionally the PAM, or a pharmaceutically acceptable salt thereof, is administered to both nostrils per dose.

Embodiment I-68. The method, compound for use, or combined preparation of any one of Embodiments I-1 to I-67, wherein the nAChR agonist, or the pharmaceutically acceptable salt thereof, and optionally the PAM, or a pharmaceutically acceptable salt thereof, is administered for at least 28 days.

Embodiment I-69. The method, compound for use, or combined preparation of any one of Embodiments I-1-67, wherein the nAChR agonist, or the pharmaceutically acceptable salt thereof, and optionally the PAM, or a pharmaceutically acceptable salt thereof, is administered for at least 3 months.

Embodiment I-70. The method, compound for use, or combined preparation of any one of Embodiments I-1 to I-69, wherein the amount of the nAChR agonist, or pharmaceutically acceptable salt thereof, administered is not systemically bioavailable.

Embodiment I-71. The method, compound for use, or combined preparation of any one of Embodiments I-1 to I-70, wherein said method does not result in undesired systemic side effects.

Embodiment I-72. The method, compound for use, or combined preparation of any one of Embodiments I-1 to I-71, wherein said method does not result in undesired psychoactive side effects.

Embodiment I-73. The method, compound for use, or combined preparation of any one of Embodiments I-1 to I-72, wherein the individual does not experience one or more side effects selected from the group consisting of overproduction of tears, cough, throat irritation, instillation site irritation, sneezing, nasopharyngitis, nasal irritation, toothache, dry mouth, and headache.

Embodiment I-74. The method, compound for use, or combined preparation of Embodiment I-73, wherein within 5 minutes-60 minutes of administration of the first dose or one or more subsequent doses of the nAChR agonist, or the pharmaceutically acceptable salt thereof, the individual does not experience one or more side effects selected from the group consisting of overproduction of tears, cough, throat irritation, instillation site irritation, sneezing, nasopharyngitis, nasal irritation, toothache, dry mouth, and headache.

Embodiment I-75. The method, compound for use, or combined preparation of Embodiment I-73 or 1-74, wherein the overproduction of tears is indicated by an increase in the individual's Schirmer score of greater than 20 mm.

Embodiment I-76. The method, compound for use, or combined preparation of any one of Embodiments I-1 to I-75, wherein the individual has undergone Lasik surgery within 2 weeks or is scheduled to undergo Lasik surgery within 2 weeks.

Embodiment I-77. The method, compound for use, or combined preparation of any one of Embodiments I-1 to I-76, wherein the PAM, or the pharmaceutically acceptable salt thereof, selectively binds to at least one of the peripheral nicotinic acetylcholine receptor subtypes selected from alpha3beta4, alpha4beta2, alpha7, and alpha3alpha5beta4.

Embodiment I-78. The method, compound for use, or combined preparation of any one of Embodiments I-1 to I-77, wherein the nAChR agonist, or a pharmaceutically acceptable salt thereof, selectively binds to at least one of the nicotinic acetylcholine receptor subtypes selected from alpha3beta4, alpha4beta2, alpha7, and alpha3alpha5beta4.

Embodiment I-79. The method, compound for use, or combined preparation of any one of Embodiments I-1 to I-78, wherein the nAChR agonist is varenicline, or a pharmaceutically acceptable salt thereof.

Embodiment I-80. The method, compound for use, or combined preparation of any one of Embodiments I-1 to I-78, wherein the nAChR agonist is compound 1, or a pharmaceutically acceptable salt thereof.

Embodiment I-81. The method, compound for use, or combined preparation of any one of Embodiments I-1 to I-80, wherein the PAM is selected from 17-beta-Estradiol, (R)-7-bromo-N-(piperidin-3-yl)benzo[b]thiophene-2-carboxamide, ivermectin, galantamine, genistein, 5-hydroxyindole, 4BP-TQS, A-86774, CCMI, levamisole, morantel, LY-2087101, mecamylamine, menthol, NS206, NS1738, NS9283, PNU-120596, RO5126946, TBS-345, dFBR, and HEPES, or a pharmaceutically acceptable salt of any of the foregoing.

Embodiment I-82. The method, compound for use, or combined preparation of Embodiment I-81, wherein the PAM is (R)-7-bromo-N-(piperidin-3-yl)benzo[b]thiophene-2-carboxamide or 3-[3-(3-Pyridinyl)-1,2,4-oxadiazol-5-yl]benzonitrile, or a pharmaceutically acceptable salt of either of the foregoing.

Embodiment I-83. The method, compound for use, or combined preparation of Embodiment I-81, wherein the PAM is (R)-7-bromo-N-(piperidin-3-yl)benzo[b]thiophene-2-carboxamide, or a pharmaceutically acceptable salt thereof.

Embodiment I-84. The method, compound for use, or combined preparation of Embodiment I-81, wherein the PAM is 3-[3-(3-Pyridinyl)-1,2,4-oxadiazol-5-yl]benzonitrile, or a pharmaceutically acceptable salt thereof.

Embodiment I-85. The method, compound for use, or combined preparation of any one of Embodiments I-1 to I-84, wherein the trigeminal nerve is activated.

Embodiment I-86. The method, compound for use, or combined preparation of any one of Embodiments I-1 to I-84, wherein the anterior ethmoidal nerve is activated.

Embodiment I-87. The method, compound for use, or combined preparation of any one of Embodiments I-1 to I-86, wherein the nasolacrimal reflex is activated.

Embodiment I-88. A pharmaceutical formulation for local administration into the nasal cavity of an individual comprising a nAChR agonist, or a pharmaceutically acceptable salt thereof, and a PAM, or a pharmaceutically acceptable salt thereof, wherein the nAChR agonist, or a pharmaceutically acceptable salt thereof, is varenicline, or a pharmaceutically acceptable

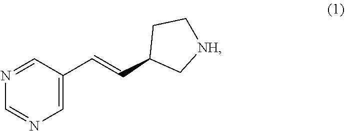 [SMILES: C(=C\[C@H]1CCNC1)/c1cncnc1]

salt thereof, or compound 1 having the structure or a pharmaceutically acceptable salt thereof, formulated for nasal administration.

Embodiment I-89. The pharmaceutical formulation of Embodiment I-88, wherein the nAChR agonist is varenicline, or a pharmaceutically acceptable salt thereof.

Embodiment I-90. The pharmaceutical formulation of Embodiment I-88, wherein the nAChR agonist is compound 1, or a pharmaceutically acceptable salt thereof.

Embodiment I-91. The pharmaceutical formulation of any one of Embodiments I-88 to I-90, comprising 5-4000 micrograms of the nAChR agonist, or a corresponding amount of a pharmaceutically acceptable salt thereof, per dose.

Embodiment I-92. The pharmaceutical formulation of any one of Embodiments I-88 to I-91, wherein the PAM is selected from the group consisting of 17-beta-Estradiol, (R)-7-bromo-N-(piperidin-3-yl)benzo[b]thiophene-2-carboxamide, ivermectin, galantamine, genistein, 5-hydroxyindole, 4BP-TQS, A-86774, CCMI, levamisole, morantel, LY-2087101, mecamylamine, menthol, NS206, NS1738, NS9283, PNU-120596, RO5126946, TBS-345, dFBR, and HEPES, or a pharmaceutically acceptable salt of any of the foregoing.

Embodiment I-93. The pharmaceutical formulation of Embodiment I-92, wherein the PAM is (R)-7-bromo-N-(piperidin-3-yl)benzo[b]thiophene-2-carboxamide or NS9283, or a pharmaceutically acceptable salt of either of the foregoing.

Embodiment I-94. The pharmaceutical formulation of any one of Embodiments I-88 to I-93, wherein the PAM alone or in combination with a nAChR agonist selectively binds to at least one of the peripheral nicotinic acetylcholine receptor subtypes selected from alpha3beta4, alpha4beta2, alpha7, and alpha3alpha5beta4.

Embodiment I-95. The pharmaceutical formulation of any one of Embodiments I-88 to I-94, wherein the pharmaceutical formulation is a liquid, suspension, aerosol, gel, ointment, dry powder, cream, paste, or balm.

Embodiment I-96. The pharmaceutical formulation of any one of Embodiments I-88 to I-95, wherein the formulation is administered into the nasal cavity by a syringe, dropper, bottle nebulizer, atomization pump, inhaler, powder spray device, vaporizer, patch, medicated stick, pipette, or jet of liquid.

Embodiment I-97. The pharmaceutical formulation of any one of Embodiments I-88 to I-96, for use in treating dry eye disease, increasing tear production, or improving ocular discomfort in an individual in need thereof.

Embodiment I-98. Use of a nAChR agonist, or a pharmaceutically acceptable salt thereof, in the manufacture of a medicament, wherein the medicament is used in a method of treating dry eye disease, increasing tear production, or reducing ocular discomfort, wherein the method is defined in any one of Embodiments I-1 to I-87.

Embodiment I-99. A kit comprising a nAChR agonist, or a pharmaceutically acceptable salt thereof, and a PAM, or a pharmaceutically acceptable salt thereof, wherein the nAChR agonist, or a pharmaceutically acceptable salt thereof, is varenicline, or a pharmaceutically acceptable salt thereof, or compound 1 having the structure

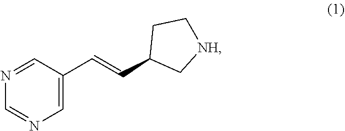 [SMILES: C(=C\[C@H]1CCNC1)/c1cncnc1]

or a pharmaceutically acceptable salt thereof.

Embodiment I-100. The kit of Embodiment I-99, wherein the nAChR agonist, or pharmaceutically acceptable salt thereof, and the PAM, or pharmaceutically acceptable salt thereof, are provided in a combination dosage form.

Embodiment I-101. The kit of Embodiment I-99, wherein the nAChR agonist, or pharmaceutically acceptable salt thereof, and the PAM, or pharmaceutically acceptable salt thereof, are provided in separate dosage forms.

Embodiment I-102. The kit of any one of Embodiments I-99 to I-101, wherein the nAChR agonist is varenicline, or a pharmaceutically acceptable salt thereof.

Embodiment I-103. The kit of any one of Embodiments I-99 to I-102, wherein the nAChR agonist is compound 1, or a pharmaceutically acceptable salt thereof.

Embodiment I-104. The kit of any one of Embodiments I-99 to I-103, comprising 5-4000 micrograms of the nAChR agonist, or a corresponding amount of a pharmaceutically acceptable salt thereof, per dose.

Embodiment I-105. The kit of any one of Embodiments I-99 to I-104, wherein the PAM is selected from the group consisting of 17-beta-Estradiol, (R)-7-bromo-N-(piperidin-3-yl)benzo[b]thiophene-2-carboxamide, ivermectin, galantamine, genistein, 5-hydroxyindole, 4BP-TQS, A-86774, CCMI, levamisole, morantel, LY-2087101, mecamylamine, menthol, NS206, NS1738, NS9283, PNU-120596, RO5126946, TBS-345, dFBR, and HEPES, or a pharmaceutically acceptable salt of any of the foregoing.

Embodiment I-106. The kit of Embodiment I-105, wherein the PAM is (R)-7-bromo-N-(piperidin-3-yl)benzo[b]thiophene-2-carboxamide or NS9283, or a pharmaceutically acceptable salt of either of the foregoing.

Embodiment I-107. The kit of any one of Embodiments I-99 to I-106, wherein the PAM alone or in combination with a nAChR agonist selectively binds to at least one of the peripheral nicotinic acetylcholine receptor subtypes selected from alpha3beta4, alpha4beta2, alpha7, and alpha3alpha5beta4.

Embodiment I-108. The kit of any one of Embodiments I-99 to I-107, for use in treating dry eye disease, increasing tear production, or improving ocular discomfort in an individual in need thereof.

Embodiment I-109. The kit of any one of Embodiments I-99 to I-108, comprising a pharmaceutical formulation, wherein the pharmaceutical formulation is a liquid, suspension, aerosol, gel, ointment, dry powder, cream, paste, or balm.

Embodiment I-110. The kit of any one of Embodiments I-99 to I-109, wherein the formulation is administered into the nasal cavity by a spray pump, syringe, dropper, bottle nebulizer, atomization pump, inhaler, powder spray device, vaporizer, patch, medicated stick, pipette, or jet of liquid.

Embodiment II-1. The method, compound for use, or combined preparation of any one of Embodiments I-1 to I-87, wherein the individual in need thereof has a blood plasma Cmax of the nAChR agonist, or a pharmaceutically acceptable salt thereof, of less than 5 ng/mL.

Embodiment II-2. The method, compound for use, or combined preparation of any one of Embodiments I-1 to I-87, or II-1, wherein 5-15 micrograms of the nAChR agonist, or a corresponding amount of a pharmaceutically acceptable salt thereof, per dose is administered to the individual.

Embodiment II-3. The method, compound for use, or combined preparation of any one of Embodiments I-1 to I-87, or II-1, wherein 50-65 micrograms of the nAChR agonist, or a corresponding amount of a pharmaceutically acceptable salt thereof, per dose is administered to the individual.

Embodiment II-4. The method, compound for use, or combined preparation of any one of Embodiments I-1 to I-87, or II-1, wherein 100-125 micrograms of the nAChR agonist, or a corresponding amount of a pharmaceutically acceptable salt thereof, per dose is administered to the individual.

Embodiment II-5. The method, compound for use, or combined preparation of any one of Embodiments I-1 to I-87, or II-1, wherein 150-300 micrograms of the nAChR agonist, or a corresponding amount of a pharmaceutically acceptable salt thereof, per dose is administered to the individual.

Embodiment II-6. The method, compound for use, or combined preparation of any one of Embodiments I-1 to I-87, or II-1, wherein 900-1200 micrograms of the nAChR agonist, or a corresponding amount of a pharmaceutically acceptable salt thereof, per dose is administered to the individual.

Embodiment II-7. The method, compound for use, or combined preparation of any one of Embodiments I-1 to I-87, or II-1, wherein 2100-2400 micrograms of the nAChR agonist, or a corresponding amount of a pharmaceutically acceptable salt thereof, per dose is administered to the individual.

Embodiment III-1. The method, compound for use, or combined preparation of any one of Embodiments I-1 to I-77, I-79 to I-87, and II-1 to II-7, wherein the nAChR agonist, or a pharmaceutically acceptable salt thereof, is an agonist of at least one of the nicotinic acetylcholine receptor subtypes selected from alpha3beta4, alpha4beta2, alpha7, alpha3alpha5beta4, and alpha4alpha6beta2.

Embodiment III-2. The method, compound for use, or combined preparation of Embodiments III-1, wherein the nAChR agonist, or a pharmaceutically acceptable salt thereof, is a full agonist of at least one of the nicotinic acetylcholine receptor subtypes selected from alpha3beta4, alpha4beta2, alpha7, alpha3alpha5beta4, and alpha4alpha6beta2.

Embodiment III-3. The method, compound for use, or combined preparation of any one of Embodiments I-1 to I-76, I-78 to I-87, II-1 to II-7, and III-1 to III-2, wherein the PAM, or the pharmaceutically acceptable salt thereof, selectively binds to at least one of the peripheral nicotinic acetylcholine receptor subtypes selected from alpha3beta4, alpha4beta2, alpha7, alpha3alpha5beta4, and alpha4alpha6beta2.

Embodiment III-4. The method, compound for use, or combined preparation of any one of Embodiments I-1 to I-77, I-79 to I-87, II-1 to II-7, and III-1 to III-3, wherein the nAChR agonist, or a pharmaceutically acceptable salt thereof, selectively binds to at least one of the nicotinic acetylcholine receptor subtypes selected from alpha3beta4, alpha4beta2, alpha7, alpha3alpha5beta4, and alpha4alpha6beta2.

Embodiment III-5. The pharmaceutical formulation of any one of Embodiments I-88 to I-93, and I-95 to I-98, wherein the nAChR agonist is an agonist of at least one of the peripheral nicotinic acetylcholine receptor subtypes selected from alpha3beta4, alpha4beta2, alpha7, alpha3alpha5beta4, and alpha4alpha6beta2.

Embodiment III-6. The pharmaceutical formulation of Embodiment III-5, wherein the nAChR agonist is a full agonist of at least one of the peripheral nicotinic acetylcholine receptor subtypes selected from alpha3beta4, alpha4beta2, alpha7, alpha3alpha5beta4, and alpha4alpha6beta2.

Embodiment III-7. The pharmaceutical formulation of any one of Embodiments I-88 to I-93, I-95 to I-98, and III-5 to III-6, wherein the PAM alone or in combination with a nAChR agonist selectively binds to at least one of the peripheral nicotinic acetylcholine receptor subtypes selected from alpha3beta4, alpha4beta2, alpha7, alpha3alpha5beta4, and alpha4alpha6beta2.

Embodiment III-8. The pharmaceutical formulation of any one of Embodiments I-88 to I-93, I-95 to I-98, and III-5 to III-7, comprising 5-15 micrograms of the nAChR agonist, or a corresponding amount of a pharmaceutically acceptable salt thereof, per dose.

Embodiment III-9. The pharmaceutical formulation of any one of Embodiments I-88 to I-93, I-95 to I-98, and III-5 to III-7, comprising 50-65 micrograms of the nAChR agonist, or a corresponding amount of a pharmaceutically acceptable salt thereof, per dose.

Embodiment III-10. The pharmaceutical formulation of any one of Embodiments I-88 to I-93, I-95 to I-98, and III-5 to III-7, comprising 100-125 micrograms of the nAChR agonist, or a corresponding amount of a pharmaceutically acceptable salt thereof, per dose.

Embodiment III-11. The pharmaceutical formulation of any one of Embodiments I-88 to I-93, I-95 to I-98, and III-5 to III-7, comprising 150-300 micrograms of the nAChR agonist, or a corresponding amount of a pharmaceutically acceptable salt thereof, per dose.

Embodiment III-12. The pharmaceutical formulation of any one of Embodiments I-88 to I-93, I-95 to I-98, and III-5 to III-7, comprising 900-1200 micrograms of the nAChR agonist, or a corresponding amount of a pharmaceutically acceptable salt thereof, per dose.

Embodiment III-13. The pharmaceutical formulation of any one of Embodiments I-88 to I-93, I-95 to I-98, and III-5 to III-7, comprising 2100-2400 micrograms of the nAChR agonist, or a corresponding amount of a pharmaceutically acceptable salt thereof, per dose.

Embodiment III-14. The kit of any one of Embodiments I-99 to I-106, and I-108 to I-110, wherein the nAChR agonist is an agonist of at least one of the peripheral nicotinic acetylcholine receptor subtypes selected from alpha3beta4, alpha4beta2, alpha7, alpha3alpha5beta4, and alpha4alpha6beta2.

Embodiment III-15. The kit of Embodiment III-14, wherein the nAChR agonist is a full agonist of at least one of the peripheral nicotinic acetylcholine receptor subtypes selected from alpha3beta4, alpha4beta2, alpha7, alpha3alpha5beta4, and alpha4alpha6beta2.

Embodiment III-16. The kit of any one of Embodiments I-99 to I-106, I-108 to I-110, and III-14 to III-15, wherein the PAM alone or in combination with a nAChR agonist selectively binds to at least one of the peripheral nicotinic acetylcholine receptor subtypes selected from alpha3beta4, alpha4beta2, alpha7, alpha3alpha5beta4, and alpha4alpha6beta2.

Embodiment IV-1. A method of treating dry eye disease, increasing tear production, or reducing ocular discomfort in an individual in need thereof, comprising administering a first dose, and optionally one or more subsequent doses, of an effective amount of a nicotinic acetylcholine receptor (nAChR) agonist, or a pharmaceutically acceptable salt thereof, and a first dose, and optionally one or more subsequent doses, of an effective amount of a positive allosteric modulator (PAM), or a pharmaceutically acceptable salt thereof, into a nasal cavity of the individual in need thereof, wherein the nAChR agonist, or a pharmaceutically acceptable salt thereof, is varenicline, or a pharmaceutically acceptable salt thereof, or compound 1 having the structure

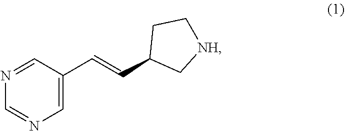 [SMILES: C(=C\[C@H]1CCNC1)/c1cncnc1]

or a pharmaceutically acceptable salt thereof, wherein the method results in the effective treatment of the individual in need thereof.

Embodiment IV-2. A compound for use in a method of treating dry eye disease, increasing tear production, or reducing ocular discomfort in an individual in need thereof, wherein the compound is a nAChR agonist, or a pharmaceutically acceptable salt thereof, wherein the method comprises administering a first dose, and optionally one or more subsequent doses, of an effective amount of a nAChR agonist, or a pharmaceutically acceptable salt thereof, and a first dose, and optionally one or more subsequent doses, of an effective amount of a PAM, or a pharmaceutically acceptable salt thereof, into a nasal cavity of the individual in need thereof, wherein the nAChR agonist, or a pharmaceutically acceptable salt thereof, is varenicline, or a pharmaceutically acceptable salt thereof, or compound 1 having the structure

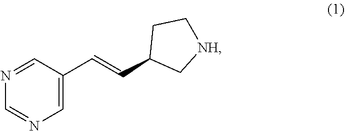 [SMILES: C(=C\[C@H]1CCNC1)/c1cncnc1]

or a pharmaceutically acceptable salt thereof.

Embodiment IV-3. A compound for use in a method of treating dry eye disease, increasing tear production, or reducing ocular discomfort in an individual in need thereof, wherein the compound is a PAM, or a pharmaceutically acceptable salt thereof, wherein the method comprises administering a first dose, and optionally one or more subsequent doses, of an effective amount of a nAChR agonist, or a pharmaceutically acceptable salt thereof, and a first dose, and optionally one or more subsequent doses, of an effective amount of a PAM, or a pharmaceutically acceptable salt thereof, into a nasal cavity of the individual in need thereof, and wherein the nAChR agonist, or a pharmaceutically acceptable salt thereof, is varenicline, or a pharmaceutically acceptable salt thereof, or compound 1 having the structure

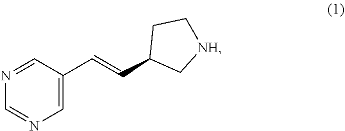 [SMILES: C(=C\[C@H]1CCNC1)/c1cncnc1]

or a pharmaceutically acceptable salt thereof.

Embodiment IV-4. A combined preparation of: (i) a nAChR agonist, or a pharmaceutically acceptable salt thereof, and (ii) a PAM, or a pharmaceutically acceptable salt thereof, for simultaneous, separate or sequential use in a method of treating dry eye disease, increasing tear production, or reducing ocular discomfort in an individual in need thereof, wherein the nAChR agonist is varenicline, compound 1 having the structure

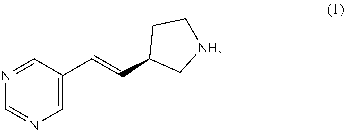 [SMILES: C(=C\[C@H]1CCNC1)/c1cncnc1]

or a pharmaceutically acceptable salt thereof, and wherein the method comprises administering a first dose, and optionally one or more subsequent doses, of an effective amount of the nAChR agonist, or pharmaceutically acceptable salt thereof, and a first dose, and optionally one or more subsequent doses, of an effective amount of the PAM, or pharmaceutically acceptable salt thereof, into a nasal cavity of the individual in need thereof.

Embodiment IV-5. The compound for use of Embodiment IV-2 or IV-3 or the combined preparation of Embodiment IV-4 wherein the method results in the effective treatment of the individual in need thereof.

Embodiment IV-6. The method of Embodiment IV-1, the compound for use of Embodiment IV-5, or the combined preparation of Embodiment IV-5, wherein the effective treatment of the individual is indicated by one or more of the tests selected from the group consisting of a) Eye Dryness score test on a visual analog scale, b) Schirmer's test, c) Corneal Fluorescein Staining test, and d) Ocular Surface Disease Index test.

Embodiment IV-7. The method, compound for use, or combined preparation of Embodiment IV-6, wherein the effective treatment is indicated by a statistically significant decrease in the individual's Eye Dryness score, and wherein the statistically significant decrease in the individual's Eye Dryness score is determined after administration to the individual of the first dose, or the optionally one or more subsequent doses, of the nAChR agonist, or a pharmaceutically acceptable salt thereof, and the first dose, or the optionally one or more subsequent doses, of the PAM, or a pharmaceutically acceptable salt thereof, wherein the individual's Eye Dryness score is compared to a) an Eye Dryness score of the individual prior to administration of the first dose of the nAChR agonist, or a pharmaceutically acceptable salt thereof, and the first dose of the PAM, or a pharmaceutically acceptable salt thereof b) an Eye Dryness score of an individual administered a control; or c) an Eye Dryness score of an individual administered a comparator compound.

Embodiment IV-8. The method, compound for use, or combined preparation of Embodiment IV-7, wherein the statistically significant decrease in the individual's Eye Dryness score is at least 15%.

Embodiment IV-9. The method, compound for use, or combined preparation of Embodiment IV-7 or IV-8, wherein the statistically significant decrease in the individual's Eye Dryness score is between 10 mm and 20 mm.

Embodiment IV-10. The method, compound for use, or combined preparation of any one of claim Embodiments IV-7 to IV-9, wherein the statistically significant decrease in the individual's Eye Dryness score is within 5 minutes of administration of the first dose of the nAChR agonist, or a pharmaceutically acceptable salt thereof, and, optionally, the first dose of the PAM, or a pharmaceutically acceptable salt thereof.

Embodiment IV-11. The method, compound for use, or combined preparation of any one of Embodiments IV-7 to IV-10, wherein the statistically significant improvement in the individual's Eye Dryness score is maintained for at least 30 minutes from administration of the first dose of the nAChR agonist, or a pharmaceutically acceptable salt thereof, and, optionally, the first dose of the PAM, or a pharmaceutically acceptable salt thereof.

Embodiment IV-12. The method, compound for use, or combined preparation of any one of Embodiments IV-7 to IV-11, wherein, between determinations of the individual's Eye Dryness score, the individual is present in an environment with reduced humidity.

Embodiment IV-13. The method, compound for use, or combined preparation of any one of Embodiments IV-7 to IV-12, comprises administering a first dose, and one or more subsequent doses, of an effective amount of a PAM, or a pharmaceutically acceptable salt thereof, and a first dose, and one or more subsequent doses, of an effective amount of a nAChR agonist, or a pharmaceutically acceptable salt thereof.

Embodiment IV-14. The method, compound for use, or combined preparation of any one of Embodiments IV-6 to IV-13, wherein the effective treatment is indicated by a statistically significant increase in the individual's Schirmer's score, and wherein the statistically significant increase in the individual's Schirmer's score in at least one eye is determined after administration to the individual of the first dose, or the optionally one or more subsequent doses, of the nAChR agonist, or a pharmaceutically acceptable salt thereof, and the first dose, or the optionally one or more subsequent doses, of the PAM, or a pharmaceutically acceptable salt thereof, wherein the individual's Schirmer's score is compared to a) a Schirmer's score of the individual prior to administration of the first dose of the nAChR agonist, or a pharmaceutically acceptable salt thereof, and the first dose of the PAM, or a pharmaceutically acceptable salt thereof; b) a Schirmer's score of an individual administered a control; or c) a Schirmer's score of an individual administered a comparator compound.

Embodiment IV-15. The method, compound for use, or combined preparation of Embodiment IV-14, wherein the statistically significant increase in the individual's Schirmer's score is at least 15%.

Embodiment IV-16. The method, compound for use, or combined preparation of Embodiments IV-14 or IV-15, wherein the statistically significant increase in the individual's Schirmer's score is between 10 mm and 20 mm.

Embodiment IV-17. The method, compound for use, or combined preparation of any one of Embodiments IV-14 to IV-16, wherein the statistically significant increase in the individual's Schirmer's score is within 5 minutes of administration of the first dose of the nAChR agonist, or a pharmaceutically acceptable salt thereof, and, optionally, the first dose of the PAM, or a pharmaceutically acceptable salt thereof.

Embodiment IV-18. The method, compound for use, or combined preparation of any one of Embodiments IV-14 to IV-17, wherein the statistically significant improvement in the individual's Schirmer's score is maintained for at least 30 minutes from administration of the first dose of the nAChR agonist, or a pharmaceutically acceptable salt thereof, and, optionally, the first dose of the PAM, or a pharmaceutically acceptable salt thereof.

Embodiment IV-19. The method, compound for use, or combined preparation of any one of Embodiments IV-14 to IV-18, wherein, between determinations of the individual's Schirmer's score, the individual is present in an environment with reduced humidity.

Embodiment IV-20. The method, compound for use, or combined preparation of any one of Embodiments IV-6 to IV-19, wherein the effective treatment is indicated by a statistically significant decrease in the individual's corneal score, and wherein the statistically significant decrease in the individual's corneal score is determined after administration to the individual of the first dose, or the optionally one or more subsequent doses, of the nAChR agonist, or a pharmaceutically acceptable salt thereof, and the first dose, or the optionally one or more subsequent doses, of the PAM, or a pharmaceutically acceptable salt thereof, wherein the individual's corneal score is compared to a) a corneal score of the individual prior to administration of the first dose of the nAChR agonist, or a pharmaceutically acceptable salt thereof, and the first dose of the PAM, or a pharmaceutically acceptable salt thereof b) a corneal score of an individual administered a control; or c) a corneal score of an individual administered a comparator compound.

Embodiment IV-21. The method, compound for use, or combined preparation of Embodiment IV-20, wherein the statistically significant decrease in the individual's corneal score is at least 15%.

Embodiment IV-22. The method, compound for use, or combined preparation of any one of Embodiments IV-20 to IV-21, wherein the statistically significant decrease in the individual's corneal score is within 5 minutes of administration of the first dose of the nAChR agonist, or a pharmaceutically acceptable salt thereof, and, optionally, the first dose of the PAM, or a pharmaceutically acceptable salt thereof.

Embodiment IV-23. The method, compound for use, or combined preparation of any one of Embodiments IV-20 to IV-22, wherein the statistically significant improvement in the individual's corneal score is maintained for at least 30 minutes from administration of the first dose of the nAChR agonist, or a pharmaceutically acceptable salt thereof, and, optionally, the first dose of the PAM, or a pharmaceutically acceptable salt thereof.

Embodiment IV-24. The method, compound for use, or combined preparation of any one of Embodiments IV-20 to IV-23, wherein, between determinations of the individual's corneal score, the individual is present in an environment with reduced humidity.

Embodiment IV-25. The method, compound for use, or combined preparation of any one of Embodiments IV-20 to IV-24, comprises administering a first dose, and one or more subsequent doses, of an effective amount of a PAM, or a pharmaceutically acceptable salt thereof, and a first dose, and one or more subsequent doses, of an effective amount of a nAChR agonist, or a pharmaceutically acceptable salt thereof.

Embodiment IV-26. The method, compound for use, or combined preparation of any one of Embodiments IV-6 to IV-25, wherein the effective treatment is indicated by a statistically significant decrease in the individual's OSDI score, and wherein the statistically significant decrease in the individual's OSDI score is determined after administration to the individual of the first dose, or the optionally one or more subsequent doses, of the nAChR agonist, or a pharmaceutically acceptable salt thereof, and the first dose, or the optionally one or more subsequent doses, of the PAM, or a pharmaceutically acceptable salt thereof, wherein the individual's OSDI score is compared to a) a OSDI score of the individual prior to administration of the first dose of the nAChR agonist, or a pharmaceutically acceptable salt thereof, and the first dose of the PAM, or a pharmaceutically acceptable salt thereof; b) a OSDI score of an individual administered a control; or c) a OSDI score of an individual administered a comparator compound.

Embodiment IV-27. The method, compound for use, or combined preparation of Embodiment IV-26, wherein the statistically significant decrease in the individual's OSDI score is at least 15%.

Embodiment IV-28. The method, compound for use, or combined preparation of any one of Embodiments IV-26 to IV-27, wherein the statistically significant decrease in the individual's OSDI score is within 5 minutes of administration of the first dose of the nAChR agonist, or a pharmaceutically acceptable salt thereof, and, optionally, the first dose of the PAM, or a pharmaceutically acceptable salt thereof.

Embodiment IV-29. The method, compound for use, or combined preparation of any one of Embodiments IV-26 to IV-28, wherein the statistically significant improvement in the individual's OSDI score is maintained for at least 30 minutes from administration of the first dose of the nAChR agonist, or a pharmaceutically acceptable salt thereof, and, optionally, the first dose of the PAM, or a pharmaceutically acceptable salt thereof.

Embodiment IV-30. The method, compound for use, or combined preparation of any one of Embodiments IV-26 to IV-29, wherein, between determinations of the individual's OSDI score, the individual is present in an environment with reduced humidity.

Embodiment IV-31. The method, compound for use, or combined preparation of any one of Embodiments IV-1 to IV-30, wherein 5-4000 micrograms of the nAChR agonist, or a corresponding amount of a pharmaceutically acceptable salt thereof, per dose is administered to the individual.

Embodiment IV-32. The method, compound for use, or combined preparation of claim any one of Embodiments IV-1 to IV-31, wherein the nAChR agonist, or a pharmaceutically acceptable salt thereof, is administered in a pharmaceutical formulation for nasal administration comprising between 1 mg/mL and 40 mg/mL of nAChR agonist, or a corresponding amount of a pharmaceutically acceptable salt thereof.

Embodiment IV-33. The method, compound for use, or combined preparation of any one of Embodiments IV-1 to IV-32, wherein the first dose, and the optionally one or more subsequent doses of the nAChR agonist, or a pharmaceutically acceptable salt thereof, is administered in a pharmaceutical formulation for nasal administration, and the total volume of the pharmaceutical formulation administered per dose of the nAChR agonist, or a pharmaceutically acceptable salt thereof, to the individual is 50 microliters-250 microliters.

Embodiment IV-34. The method, compound for use, or combined preparation of any one of Embodiments IV-1 to IV-33, wherein the first dose, and the optionally one or more subsequent doses, of the nAChR agonist, or a pharmaceutically acceptable salt thereof, is administered in a pharmaceutical formulation for nasal administration, and the total volume of the pharmaceutical formulation administered per nostril of the nAChR agonist, or a pharmaceutically acceptable salt thereof, to the individual is 50 microliters-250 microliters.

Embodiment IV-35. The method, compound for use, or combined preparation of any one of Embodiments IV-1 to IV-34, wherein the PAM, or the pharmaceutically acceptable salt thereof, and the nAChR agonist, or the pharmaceutically acceptable salt thereof, are administered to the individual in need thereof in separate dosage forms.

Embodiment IV-36. The method, compound for use, or combined preparation of any one of Embodiments IV-1 to IV-34, wherein the PAM, or the pharmaceutically acceptable salt thereof, and the nAChR agonist, or the pharmaceutically acceptable salt thereof, are administered to the individual in need thereof in a combined dosage form.

Embodiment IV-37. The method, compound for use, or combined preparation of any one of Embodiments IV-1 to IV-35, wherein the PAM, or the pharmaceutically acceptable salt thereof, is administered to the individual in need thereof administered after the nAChR agonist, or the pharmaceutically acceptable salt thereof.

Embodiment IV-38. The method, compound for use, or combined preparation of any one of Embodiments IV-1 to IV-35, wherein the PAM, or the pharmaceutically acceptable salt thereof, is administered to the individual in need thereof before the nAChR agonist, or the pharmaceutically acceptable salt thereof.

Embodiment IV-39. The method, compound for use, or combined preparation of any one of Embodiments IV-1 to IV-36, wherein the PAM, or the pharmaceutically acceptable salt thereof, and the nAChR agonist, or the pharmaceutically acceptable salt thereof, are administered to the individual in need thereof at the same time.

Embodiment IV-40. The method, compound for use, or combined preparation of any one of Embodiments IV-1 to IV-39, wherein a dose of the nAChR agonist, or the pharmaceutically acceptable salt thereof, and optionally a dose of the PAM, or a pharmaceutically acceptable salt thereof, is administered to the individual in need thereof one to four times daily after the first day of administration.

Embodiment IV-41. The method, compound for use, or combined preparation of any one of Embodiments IV-1 to IV-40, wherein a) the dose frequency of the nAChR agonist, or the pharmaceutically acceptable salt thereof, orb) the total amount of the nAChR agonist, or the corresponding amount of the pharmaceutically acceptable salt thereof, per dose is reduced over time.

Embodiment IV-42. The method, compound for use, or combined preparation of Embodiment IV-41, wherein each dose of the nAChR agonist, or the pharmaceutically acceptable salt thereof, comprises more than one administration of the nAChR agonist, or the pharmaceutically acceptable salt thereof, into the nasal cavity of the individual, and wherein the reduction of the total amount of the nAChR agonist, or the corresponding amount of pharmaceutically acceptable salt thereof, per dose over time is accomplished by reducing the number of administrations of the nAChR agonist, or the pharmaceutically acceptable salt thereof, into the nasal cavity of the individual.

Embodiment IV-43. The method, compound for use, or combined preparation of any one of Embodiments IV-1 to IV-42, wherein the nAChR agonist, or the pharmaceutically acceptable salt thereof, and optionally the PAM, or a pharmaceutically acceptable salt thereof, is administered to one nostril per dose.

Embodiment IV-44. The method, compound for use, or combined preparation of any one of Embodiments IV-1 to IV-42, wherein the nAChR agonist, or the pharmaceutically acceptable salt thereof, and optionally the PAM, or a pharmaceutically acceptable salt thereof, is administered to both nostrils per dose.

Embodiment IV-45. The method, compound for use, or combined preparation of any one of Embodiments IV-1 to IV-44, wherein the nAChR agonist, or the pharmaceutically acceptable salt thereof, and optionally the PAM, or a pharmaceutically acceptable salt thereof, is administered for at least 28 days.

Embodiment IV-46. The method, compound for use, or combined preparation of any one of Embodiments IV-1 to IV-44, wherein the nAChR agonist, or the pharmaceutically acceptable salt thereof, and optionally the PAM, or a pharmaceutically acceptable salt thereof, is administered for at least 3 months.

Embodiment IV-47. The method, compound for use, or combined preparation of any one of Embodiments IV-1 to IV-46, wherein the amount of the nAChR agonist, or pharmaceutically acceptable salt thereof, administered is not systemically bioavailable.

Embodiment IV-48. The method, compound for use, or combined preparation of any one of Embodiments IV-1 to IV-47, wherein said method does not result in undesired systemic side effects.

Embodiment IV-49. The method, compound for use, or combined preparation of any one of Embodiments IV-1 to IV-48, wherein said method does not result in undesired psychoactive side effects.

Embodiment IV-50. The method, compound for use, or combined preparation of any one of Embodiments IV-1 to IV-49, wherein the individual does not experience one or more side effects selected from the group consisting of overproduction of tears, cough, throat irritation, instillation site irritation, sneezing, nasopharyngitis, nasal irritation, toothache, dry mouth, and headache.

Embodiment IV-51. The method, compound for use, or combined preparation of Embodiment IV-50, wherein within 5 minutes-60 minutes of administration of the first dose or one or more subsequent doses of the nAChR agonist, or the pharmaceutically acceptable salt thereof, the individual does not experience one or more side effects selected from the group consisting of overproduction of tears, cough, throat irritation, instillation site irritation, sneezing, nasopharyngitis, nasal irritation, toothache, dry mouth, and headache.

Embodiment IV-52. The method, compound for use, or combined preparation of Embodiment IV-50 or IV-51, wherein the overproduction of tears is indicated by an increase in the individual's Schirmer score of greater than 20 mm.

Embodiment IV-53. The method, compound for use, or combined preparation of any one of Embodiments IV-1 to IV-52, wherein the individual has undergone Lasik surgery within 2 weeks or is scheduled to undergo Lasik surgery within 2 weeks.

Embodiment IV-54. The method, compound for use, or combined preparation of any one of Embodiments IV-1 to IV-53, wherein the PAM, or the pharmaceutically acceptable salt thereof, selectively binds to at least one of the peripheral nicotinic acetylcholine receptor subtypes selected from alpha3beta4, alpha4beta2, alpha7, alpha3alpha5beta4, and alpha4alpha6beta2.

Embodiment IV-55. The method, compound for use, or combined preparation of any one of Embodiments IV-1 to IV-54, wherein the nAChR agonist, or a pharmaceutically acceptable salt thereof, selectively binds to at least one of the nicotinic acetylcholine receptor subtypes selected from alpha3beta4, alpha4beta2, alpha7, alpha3alpha5beta4, and alpha4alpha6beta2.

Embodiment IV-56. The method, compound for use, or combined preparation of any one of Embodiments IV-1 to IV-55, wherein the nAChR agonist is varenicline, or a pharmaceutically acceptable salt thereof.

Embodiment IV-57. The method, compound for use, or combined preparation of any one of Embodiments IV-1 to IV-55, wherein the nAChR agonist is compound 1, or a pharmaceutically acceptable salt thereof.

Embodiment IV-58. The method, compound for use, or combined preparation of any one of Embodiments IV-1 to IV-57, wherein the PAM is selected from 17-beta-Estradiol, (R)-7-bromo-N-(piperidin-3-yl)benzo[b]thiophene-2-carboxamide, ivermectin, galantamine, genistein, 5-hydroxyindole, 4BP-TQS, A-86774, CCMI, levamisole, morantel, LY-2087101, mecamylamine, menthol, NS206, NS1738, NS9283, PNU-120596, RO5126946, TB S-345, dFBR, and HEPES, or a pharmaceutically acceptable salt of any of the foregoing.

Embodiment IV-59. The method, compound for use, or combined preparation of Embodiment IV-57, wherein the PAM is (R)-7-bromo-N-(piperidin-3-yl)benzo[b]thiophene-2-carboxamide or 3-[3-(3-Pyridinyl)-1,2,4-oxadiazol-5-yl]benzonitrile, or a pharmaceutically acceptable salt of either of the foregoing.

Embodiment IV-60. The method, compound for use, or combined preparation of Embodiment IV-58, wherein the PAM is (R)-7-bromo-N-(piperidin-3-yl)benzo[b]thiophene-2-carboxamide, or a pharmaceutically acceptable salt thereof.

Embodiment IV-61. The method, compound for use, or combined preparation of Embodiment IV-58, wherein the PAM is 3-[3-(3-Pyridinyl)-1,2,4-oxadiazol-5-yl]benzonitrile, or a pharmaceutically acceptable salt thereof.

Embodiment IV-62. The method, compound for use, or combined preparation of any one of Embodiments IV-1 to IV-61, wherein the trigeminal nerve is activated.

Embodiment IV-63. The method, compound for use, or combined preparation of any one of Embodiments IV-1 to IV-61, wherein the anterior ethmoidal nerve is activated.

Embodiment IV-64. The method, compound for use, or combined preparation of any one of Embodiments IV-1 to IV-63, wherein the nasolacrimal reflex is activated.

Embodiment IV-65. The method, compound for use, or combined preparation of any one of Embodiments IV-1 to IV-64, wherein the individual in need thereof has a blood plasma Cmax of the nAChR agonist, or a pharmaceutically acceptable salt thereof, of less than 5 ng/mL.

Embodiment IV-66. The method, compound for use, or combined preparation of any one of Embodiments IV-1 to IV-65, wherein the nAChR agonist, or a pharmaceutically acceptable salt thereof, is an agonist of at least one of the nicotinic acetylcholine receptor subtypes selected from alpha3beta4, alpha4beta2, alpha7, alpha3alpha5beta4, and alpha4alpha6beta2.

Embodiment IV-67. A pharmaceutical formulation for local administration into the nasal cavity of an individual comprising a nAChR agonist, or a pharmaceutically acceptable salt thereof, and a PAM, or a pharmaceutically acceptable salt thereof, wherein the nAChR agonist, or a pharmaceutically acceptable salt thereof, is varenicline, or a pharmaceutically acceptable salt thereof, or compound 1 having the structure

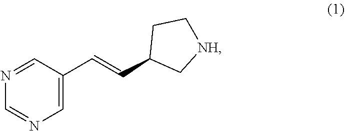 [SMILES: C(=C\[C@H]1CCNC1)/c1cncnc1]

or a pharmaceutically acceptable salt thereof, formulated for nasal administration.

Embodiment IV-68. The pharmaceutical formulation of Embodiment IV-67, wherein the nAChR agonist is varenicline, or a pharmaceutically acceptable salt thereof.

Embodiment IV-69. The pharmaceutical formulation of Embodiment IV-67, wherein the nAChR agonist is compound 1, or a pharmaceutically acceptable salt thereof.

Embodiment IV-70. The pharmaceutical formulation of any one of Embodiments IV-67 to IV-69, comprising 5-4000 micrograms of the nAChR agonist, or a corresponding amount of a pharmaceutically acceptable salt thereof, per dose.

Embodiment IV-71. The pharmaceutical formulation of any one of Embodiments IV-67 to IV-70, wherein the PAM is selected from the group consisting of 17-beta-Estradiol, (R)-7-bromo-N-(piperidin-3-yl)benzo[b]thiophene-2-carboxamide, ivermectin, galantamine, genistein, 5-hydroxyindole, 4BP-TQS, A-86774, CCMI, levamisole, morantel, LY-2087101, mecamylamine, menthol, NS206, NS1738, NS9283, PNU-120596, RO5126946, TBS-345, dFBR, and HEPES, or a pharmaceutically acceptable salt of any of the foregoing.

Embodiment IV-72. The pharmaceutical formulation of any one of Embodiments IV-67 to IV-71, wherein the PAM is (R)-7-bromo-N-(piperidin-3-yl)benzo[b]thiophene-2-carboxamide or NS9283, or a pharmaceutically acceptable salt of either of the foregoing.

Embodiment IV-73. The pharmaceutical formulation of any one of Embodiments IV-67 to IV-72, wherein the PAM alone or in combination with a nAChR agonist selectively binds to at least one of the peripheral nicotinic acetylcholine receptor subtypes selected from alpha3beta4, alpha4beta2, alpha7, alpha3alpha5beta4, and alpha4alpha6beta2.

Embodiment IV-74. The pharmaceutical formulation of any one of Embodiments IV-67 to IV-73, wherein the pharmaceutical formulation is a liquid, suspension, aerosol, gel, ointment, dry powder, cream, paste, or balm.

Embodiment IV-75. The pharmaceutical formulation of any one of Embodiments IV-67 to IV-74, wherein the formulation is administered into the nasal cavity by a syringe, dropper, bottle nebulizer, atomization pump, inhaler, powder spray device, vaporizer, patch, medicated stick, pipette, or jet of liquid.

Embodiment IV-76. The pharmaceutical formulation of any one of Embodiments IV-67 to IV-75, for use in treating dry eye disease, increasing tear production, or improving ocular discomfort in an individual in need thereof.

Embodiment IV-77. The pharmaceutical formulation of any one of Embodiments IV-67 to IV-76, wherein the nAChR agonist is an agonist of at least one of the peripheral nicotinic acetylcholine receptor subtypes selected from alpha3beta4, alpha4beta2, alpha7, alpha3alpha5beta4, and alpha4alpha6beta2.

Embodiment IV-78. Use of a nAChR agonist, or a pharmaceutically acceptable salt thereof, in the manufacture of a medicament, wherein the medicament is used in a method of treating dry eye disease, increasing tear production, or reducing ocular discomfort, wherein the method is defined in any one of Embodiments IV-1 to IV-66.

Embodiment IV-79. A kit comprising a nAChR agonist, or a pharmaceutically acceptable salt thereof, and a PAM, or a pharmaceutically acceptable salt thereof, wherein the nAChR agonist, or a pharmaceutically acceptable salt thereof, is varenicline, or a pharmaceutically acceptable salt thereof, or compound 1 having the structure

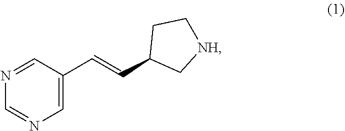 [SMILES: C(=C\[C@H]1CCNC1)/c1cncnc1]

or a pharmaceutically acceptable salt thereof.

Embodiment IV-80. The kit of Embodiment IV-79, wherein the nAChR agonist, or pharmaceutically acceptable salt thereof, and the PAM, or pharmaceutically acceptable salt thereof, are provided in a combination dosage form.

Embodiment IV-81. The kit of Embodiment IV-79, wherein the nAChR agonist, or pharmaceutically acceptable salt thereof, and the PAM, or pharmaceutically acceptable salt thereof, are provided in separate dosage forms.

Embodiment IV-82. The kit of any one of Embodiments IV-79 to IV-81, wherein the nAChR agonist is varenicline, or a pharmaceutically acceptable salt thereof.

Embodiment IV-83. The kit of any one of Embodiments IV-79 to IV-82, wherein the nAChR agonist is compound 1, or a pharmaceutically acceptable salt thereof.

Embodiment IV-84. The kit of any one of Embodiments IV-79 to IV-83, comprising 5-4000 micrograms of the nAChR agonist, or a corresponding amount of a pharmaceutically acceptable salt thereof, per dose.

Embodiment IV-85. The kit of any one of Embodiments IV-79 to IV-84, wherein the PAM is selected from the group consisting of 17-beta-Estradiol, (R)-7-bromo-N-(piperidin-3-yl)benzo[b]thiophene-2-carboxamide, ivermectin, galantamine, genistein, 5-hydroxyindole, 4BP-TQS, A-86774, CCMI, levamisole, morantel, LY-2087101, mecamylamine, menthol, NS206, NS1738, NS9283, PNU-120596, RO5126946, TBS-345, dFBR, and HEPES, or a pharmaceutically acceptable salt of any of the foregoing.

Embodiment IV-86. The kit of Embodiment IV-85, wherein the PAM is (R)-7-bromo-N-(piperidin-3-yl)benzo[b]thiophene-2-carboxamide or NS9283, or a pharmaceutically acceptable salt of either of the foregoing.

Embodiment IV-87. The kit of any one of Embodiments IV-79 to IV-86, for use in treating dry eye disease, increasing tear production, or improving ocular discomfort in an individual in need thereof.

Embodiment IV-88. The kit of any one of Embodiments IV-79 to IV-87, comprising a pharmaceutical formulation, wherein the pharmaceutical formulation is a liquid, suspension, aerosol, gel, ointment, dry powder, cream, paste, or balm.

Embodiment IV-89. The kit of any one of Embodiments IV-79 to IV-88, wherein the formulation is administered into the nasal cavity by a spray pump, syringe, dropper, bottle nebulizer, atomization pump, inhaler, powder spray device, vaporizer, patch, medicated stick, pipette, or jet of liquid.

Embodiment IV-90. The kit of any one of Embodiments IV-79 to IV-89, wherein the PAM alone or in combination with a nAChR agonist selectively binds to at least one of the peripheral nicotinic acetylcholine receptor subtypes selected from alpha3beta4, alpha4beta2, alpha7, alpha3alpha5beta4, and alpha4alpha6beta2.

Embodiment IV-91. The kit of any one of Embodiments IV-79 to IV-90, wherein the nAChR agonist is an agonist of at least one of the peripheral nicotinic acetylcholine receptor subtypes selected from alpha3beta4, alpha4beta2, alpha7, alpha3alpha5beta4, and alpha4alpha6beta2.

### EXAMPLES

### Example 1: In Vitro Activity of nAChR Agonists and PAMs Combinations on Human Alpha4beta2 nAChR Expressed in Xenopus Oocytes

This example describes an experiment to characterize the effects of compound 1 hemi-galactarate dihydrate and varenicline tartrate at the human neuronal nicotinic acetylcholine receptors (nAChRs) alpha4beta2 expressed in Xenopus oocytes. The experiment also evaluated the agonist activity and receptor desensitization effects of two PAMs (Br-PBTC, (R)-7-bromo-N-(piperidin-3-yl)benzo[b]thiophene-2-carboxamide; NS-9283, 3-[3-(3-Pyridinyl)-1,2,4-oxadiazol-5-yl]benzonitrile). The data provides evidence that the use of a PAM potentiates the activity of varenicline tartrate, reduces desensitization of the alpha4beta2 receptor, and enhances recovery of the receptor response.

Determination of the concentration activation curve to compound 1 hemi-galactarate dihydrate in absence and presence of the positive allosteric modulators Br-PBTC and/or NS-9283 (at 1 μM) reveals a significant shift to the left accompanied for the Br-PBTC by an increase of the maximal evoked current. These data confirm that both compounds act as positive allosteric modulators at the human alpha4beta2 nAChRs in the presence of compound 1 hemi-galactarate dihydrate.

While, due to the long-lasting binding of varenicline tartrate at the alpha4beta2, the same experimental protocol could not be used, the confirmation of the PAM effects was obtained using alternative approaches which also revealed that potentiation is independent from the order of agonist or PAM applications.

Sustained exposure to an agonist is known to desensitize the alpha4beta2 nAChRs and recovery of the responses depends upon multiple factors. Measurements of the responses evoked by 1 mM compound 1 hemi-galactarate dihydrate or 300 μM varenicline tartrate and recovery from a one hour exposure to these compounds revealed that while compound 1 hemi-galactarate dihydrate causes no long-lasting desensitization in this model, application of varenicline tartrate caused a profound inhibition that could not be removed even after a three-hour wash period.

Effects of the Br-PBTC and NS-9283 were examined both on the responses evoked by 1 mM compound 1 hemi-galactarate dihydrate and its recovery and/or on 300 μM varenicline tartrate. While co-application of the modulator had minimal effect on the profile of compound 1 hemi-galactarate dihydrate desensitization and recovery in this model, a marked difference was observed for varenicline tartrate in presence of Br-PBTC allowing almost complete recovery within 3 hours.

1.1 Test compounds
Compound 1 hemi-galactarate dihydrate
Varenicline tartrate
Br-PBTC
NS-9283

### 1.2 Reference

Acetylcholine chloride
100 mM stock solution in bi-distilled water
   1.3 Test system
Apparatus: Automated Injection and Recording Apparatus, HiQScreen
Species of donor animals: Xenopus
Strain of animals: Laevis
Origin (supplier) of animals: HiQScreen Sàrl, Geneva
Body Weight of animals: 150 g to 300 g
Animal maintenance 3 to 5 per vivarium
Room temperature: 20-22° C.
Lighting times: Vivarium were exposed to daylight with additional artificial lighting (6 am-8 pm in summer; 8 am-7 pm in winter).
Food: Food pellets, ad libitum
Source of biological material
and isolation procedure: Abdominal incision to remove ovaries
Experimental conditions in Oocyte incubation: Barth solution containing in mM NaCl 88,
vitro: KCl 1, NaHCO₃2.4, HEPES 10, MgSO₄.7H₂O 0.82, Ca(NO₃)₂.4H₂O 0.33, CaCl₂.6H₂O 0.41, at pH 7.4, and supplemented with 100 unit/ml penicillin
   Oocyte recording: OR2 medium containing in mM: NaCl 88.5, KCl 2.5, HEPES 5, CaCl₂.2H₂O 1.8, MgCl₂.6H₂O 1, pH 7.4.
Test procedure: Two-electrode voltage clamp, using proprietary automated system equipped with a Geneclamp amplifier (Axon Instrument, Foster City, Calif., USA), or HiClamp (Multichannel Systems, Germany)
   Abbreviations: Ach, Acetylcholine; nAChR, Nicotinic acetylcholine receptor; SEM, Standard error of the mean; PAM, Positive allosteric modulator

### Oocytes Preparation

All experiments were carried out at human nAChRs expressed in Xenopus oocytes using the method of cDNA expression. Xenopus oocytes were prepared and injected using standard procedures.

Briefly, ovaries were harvested from Xenopus Laevis females that have been deeply anesthetized by cooling at 4° C. and Tricaine methanesulfonate (MS-222 at a concentration of 150 mg/L) in sodium bicarbonate (300 mg/L). Once anesthetized the animal was decapitated and pithed following the rules of animal rights from the Geneva canton. A small piece of ovary was isolated for immediate preparation while the remaining part was placed at 4° C. in a sterile Barth solution containing in mM NaCl 88, KCl 1, NaHCO₃2.4, HEPES 10, MgSO₄.7H₂O 0.82, Ca(NO₃)₂.4H₂O 0.33, CaCl₂.6H₂O 0.41, at pH 7.4, and supplemented with 20 μg/ml of kanamycin, 100 unit/ml penicillin. All recordings were performed at 18° C. and cells superfused with OR2 medium containing in mM: NaCl 88, KCl 2.5, HEPES 5, CaCl₂.2H₂O 1.8, MgCl₂.6H₂O 1, pH 7.4.

### Electrophysiological Recordings

Currents evoked by ACh or other agonists were recorded using an automated process equipped with standard two-electrode voltage-clamp configuration (TEVC). Data were captured and analyzed using a HiQScreen proprietary data acquisition and analysis software running under Matlab (Mathworks Inc.).

### Agonist Preparation

ACh was prepared as a concentrated stock solution (10⁻¹M) in water and then diluted in the recording medium to obtain the desired test concentration. Compounds were dissolved in OR2 as stock solution (1 mM) and the final dilution to the desired concentration was made on the day of the experiment.

### Data Analysis and Statistics

For statistical analysis values were computed either with Excel (Microsoft) or Matlab (mathworks Inc.). To obtain mean measurements with standard deviations, all experiments were carried out using at least three cells.

### Experimental Procedures

Injections of cDNAs encoding for the human alpha4beta2, were performed in at least 95 oocytes were performed using a proprietary automated injection device (Hogg et al., J. Neurosci. Methods, 2008) and receptor expression examined at least two days later. Oocytes were poked with two electrodes and their membrane potential maintained, unless indicated, at −80 mV throughout the experiment.

A protocol to determine the allosteric modulation at the alpha4beta2 nAChRs in the oocytes expressing the receptor was developed. Oocytes expressing alpha4beta2 nAChRs were exposed to increasing concentrations of ACh while the response was evaluated. This was repeated with the addition of a fixed concentration of the allosteric modulator. The current evoked from the exposure to the agonist with the modulator was recorded and compared to currents evoked in the absence of the modulator.

1.4 Effects of a PAM at the alpha4beta2 nAChR Response to Compound 1 Hemi-Galactarate Dihydrate

To assess the effects of positive allosteric modulators (Br-PBTC and NS-9283) a protocol of determination of the concentration activation curve in absence and presence of the PAM was designed. These data illustrate that exposure to 1 μM Br-PBTC enhances the amplitude of the current evoked by compound 1 hemi-galactarate dihydrate.

Plot of the peak current amplitude recorded in absence and presence of the modulator (PAM) further reveals that the concentration activation curve to compound 1 hemi-galactarate dihydrate is shifted to the left and currents are increased in presence of 1 μM Br-PBTC. These data confirm that Br-PBTC acts as a positive modulator at the human alpha4beta2 nAChRs in the presence of compound 1 hemi-galactarate dihydrate.

To assess the positive allosteric modulator effects of NS-9283 at the compound 1 hemi-galactarate dihydrate the same experimental protocol was conducted using 1 μM of the modulator. Results from these experiments illustrate that this compound also acts as a positive allosteric modulator in the presence of compound 1 hemi-galactarate dihydrate. However, exposure to this compound caused no significant increase of the maximal current evoked by compound 1 hemi-galactarate dihydrate indicating that Br-PBTC and NS-9283 are not acting in an identical manner.

1.5 Effects of a PAM at the alpha4beta2 nAChR Response to Varenicline Tartrate

When the same experimental protocols were repeated with varenicline tartrate, technical difficulties were observed in all the cells tested. A large signal was observed upon application of the modulator. Similar results were observed in all the cells tested. The inward current observed on application of the modulator suggests that varenicline tartrate is probably still bound to the receptor and that exposure to the PAM is unable to activate the receptors.

Due to these technical difficulties, the effects of PAMs on the concentration activation curve by varenicline tartrate could not be determined using the experimental protocol.

### 1.6 Effects of Application on the PAM Modulation

To further evaluate the PAM effects, the modulation was measured either as a first application and compared versus the subsequent agonist evoked response or, vice versa, by first exposing the cell to the agonist alone and then to the PAM and the agonist applied during a sustained PAM exposure.

As a first step, the PAM effects were determined at the response evoked by 3 μM ACh, a concentration that is in the lower range of the concentration activation curve where the maximal PAM effects can be revealed.

The same experiment was then conducted for compound 1 hemi-galactarate dihydrate as the agonist and Br-PBTC as the modulator or NS-9283 as the modulator. Altogether, these data illustrate that potentiation by the modulator is independent on the exposure protocol and that the prior exposure to the agonist does not preclude the activity of the positive allosteric modulator, for at least compound 1 hemi-galactarate dihydrate and BR-PBTC or NS-9283.

1.7 Recovery from Sustained Agonist Exposure

To mimic the patient conditions, recovery from a sustained exposure to the agonist (compound 1 hemi-galactarate dihydrate or varenicline tartrate) had previously be examined. Results from these experiments clearly revealed that whereas varenicline tartrate causes a major desensitization of the alpha4beta2 receptors, exposure to compound 1 hemi-galactarate dihydrate caused only a modest inhibition followed with a progressive increase of the evoked response as a function of time in these models.

Determination of the currents evoked by compound 1 hemi-galactarate dihydrate in a large number of cells in control conditions and at different recovery times up to 18 hours reveal that compound 1 hemi-galactarate dihydrate does not cause a long-lasting inhibition of the alpha4beta2 nAChRs in these models.

In contrast, exposure to one hour varenicline tartrate at 300 μM caused a sustained inhibition and no significant recovery was observed over a 3 hours' wash.

### 1.8 Effects of the Modulators on the Long-Time Agonist Exposure

The availability of efficient positive allosteric modulators offers the possibility to assess if exposure to a PAM modifies the desensitization and recovery time course. To determine the putative effects of Br-PBTC and/or NS-9283, experiments similar to those previously conducted were conducted.

### 1.8.1 Modulators and Desensitization by 1 mM Compound 1 Hemi-Galactarate Dihydrate

To evaluate the effects of two modulators, namely the Br-PBTC and NS-9283, at the human alpha4beta2 receptors experiments were designed using an exposure similar to previously conducted in absence of the modulators. Typically, oocytes injected with the human alpha4beta2, in a 1:1 ratio, of the corresponding cDNA's were tested for their response to a test pulse 1 mM compound 1 hemi-galactarate dihydrate applied during 15 seconds in presence of 1 μM modulator.

Cells displaying significant currents were then exposed to 1 hour of 1 mM compound 1 hemi-galactarate dihydrate in presence of 1 μM of the modulator and the medium was then replaced with control. Following a recovery period of 1, 3 and 18 hours, cells were challenged again with the test pulse of 1 mM compound 1 hemi-galactarate dihydrate and their response was recorded using the same conditions as in the control.

### 1.8.2 Modulators and Desensitization by 300 μM Varenicline Tartrate

To examine the effects of the two modulators, Br-PBTC and NS-9283, on the responses evoked by varenicline tartrate at the human alpha4beta2 receptors the same experimental protocols were conducted.

Examination of the varenicline tartrate response time course showed significant differences between the control conditions and in presence of the modulators. Average currents recorded in number of cells measured in control, or presence of 1 μM NS-9283 or 1 μM Br-PBTC were recorded. Whereas, the amplitude and time course of the responses observed in presence of 1 μM Br-PBTC are markedly different, caution should be maintained as these responses were not recorded at the same time in the same batches of cells.

### CONCLUSION

Determination of the concentration activation profile for compound 1 hemi-galactarate dihydrate in absence or presence of Br-PBTC and/or NS-9283 confirmed that both PAMs are able to potentiate the agonistic activity of compound 1 hemi-galactarate dihydrate causing an increase in the receptor sensitivity. A difference was, however, observed between the two modulators with an increase of the amplitude of the current evoked by a saturating concentration of agonist observed only for Br-PBTC.

Effects of sustained exposure (1 hour) to 1 mM compound 1 hemi-galactarate dihydrate or 300 μM varenicline tartrate were examined by monitoring first the amplitude of the evoked currents at the onset of the exposure and the by the recovery of the responses after different wash periods ranging up to 18 hours for compound 1 hemi-galactarate dihydrate. These experiments revealed that even exposure to a concentration of compound 1 hemi-galactarate dihydrate as high as 1 mM caused no long-lasting inhibition and that almost full recovery was observed after 3 hours in this model. Furthermore, a significant increase of the response was observed after 18 hours suggesting a possible up-regulation of the receptors.

In contrast, exposure to 300 μM varenicline tartrate, which evoked only a fraction of the maximal ACh-evoked current, caused a major inhibition of the subsequent response with absence of recovery even after a wash of 3 hours.

When the same experiments were repeated with 300 μM varenicline tartrate in presence of 1 μM Br-PBTC a different picture emerged. First, the amplitude of the current evoked by varenicline tartrate were largely enhanced by the presence of this modulator and, second, almost full recovery of the response was observed after a wash of 3 hours. On the contrary, while application of 1 μM NS-9283 also enhanced the response to varenicline tartrate, exposure to this compound failed to significantly modify the recovery time course.

Altogether, these data reveal that sustained exposure to agonists such as compound 1 hemi-galactarate dihydrate or varenicline tartrate caused distinct desensitization of the receptors and that recovery from varenicline tartrate desensitization was modified by the use of Br-PBTC. Specifically, these data illustrate that functional properties of the human alpha4beta2 nAChRs can readily be examined in Xenopus oocytes and that long-lasting desensitization caused by varenicline tartrate could be partially reversed by the use of a positive allosteric modulator such as Br-PBTC.

### Example 2: In Vitro Activity of nAChR Agonists On Various nAChR Subtypes

This example describes experiments to characterize the effects of varenicline tartrate and compound 1 hemi-galactarate dihydrate at the human neuronal nicotinic acetylcholine receptors (nAChRs) alpha3beta4, alpha3alpha5beta4, alpha4beta2 and alpha7 expressed in Xenopus oocytes.

Determination of the agonistic effects of compound 1 hemi-galactarate dihydrate at the alpha3beta4, alpha3alpha5beta4, alpha4beta2 and alpha7 receptors revealed that this compound acts, in the 10 to 100 μM range, as an agonist at these receptors. Noticeable differences were, however, observed in function of the receptor subtype. Namely, compound 1 hemi-galactarate dihydrate was a poor agonist at the human alpha7 receptors evoking at 300 μM only 24 percent of the maximal ACh-evoked current. In contrast, data from alpha4beta2 revealed that compound 1 hemi-galactarate dihydrate evoked currents larger than the maximal ACh responses by about 2 fold. Moreover, exposure to compound 1 hemi-galactarate dihydrate enhanced the subsequent ACh-evoked currents. Data recorded at alpha3beta4 and at alpha3alpha5beta4 revealed that compound 1 hemi-galactarate dihydrate acts as an agonist at these receptors evoking about 94 percent of the maximal ACh-evoked currents.

Determination of the agonistic properties of varenicline tartrate at the human alpha3beta4, alpha3alpha5beta4, alpha4beta2 and alpha7 nAChRs, confirmed that this compound acts as a partial agonist at the alpha3beta4, alpha3alpha5beta4 and alpha4beta2 with EC50's in the low micromolar range. Results obtained at the human alpha7 receptors confirmed that varenicline tartrate acts as a full agonist at this receptor subtype with an EC50 at about 11 μM.

Desensitization experiments conducted alpha3beta4, alpha3alpha5beta4, alpha4beta2 and alpha7 nAChRs revealed marked differences in function of the receptor subtypes. Whereas alpha3beta4 and alpha7 receptors were insensitive to sustained exposure of compound 1 hemi-galactarate dihydrate for concentrations up to 300 nM, a marked inhibition was observed for at alpha3alpha5beta4 and alpha4beta2 receptors. Inhibition reached, however, at the highest concentration tested a plateau level suggesting that this compound might cause only partial desensitization within the experimental conditions. Desensitization experiments conducted at alpha3beta4, alpha3alpha5beta4, alpha4beta2 and alpha7 nAChRs revealed the marked differences that can be observed between these different receptor subtypes. Namely, while varenicline tartrate inhibited in the nanomolar range up to 78% of the alpha4beta2 receptors, exposure to the same concentrations and duration had only a very modest effect at the alpha3beta4 receptors. Co-expression of the alpha5 receptors in the alpha3alpha5beta4 combination increased, however, the sensitivity to varenicline tartrate, causing up to 45% inhibition. The observed plateau effect of varenicline tartrate at this receptor subtype is in good agreement with its partial agonist activity. Importantly, exposure to varenicline tartrate of the alpha7 nAChRs also caused inhibition for concentrations in the hundreds of nanomolar range. Desensitization caused by 300 μM varenicline tartrate applied during three minutes at the alpha3beta4 or alpha4beta2 nAChRs, confirmed the differences in sensitivity between these two subtypes. Namely, while varenicline tartrate evoked only about 10-20% of the ACh response at the alpha4beta2, a larger current amplitude was observed at the alpha3beta4 receptors. The absence of recovery observed for period as long as 36 minutes confirmed the long lasting desensitization caused by exposure to varenicline tartrate.

### Observations and Measurements

### Oocytes Preparation

See Example 1.

Electrophysiological recordings

See Example 1.

### Agonist Preparation

See Example 1. The test concentrations of compound 1 hemi-galactarate and varenicline tartrate were (in micromolar) 0.1, 0.3, 1, 3, 10, 30, 100, and 300. The test concentration of acetylcholine chloride was 1280 micromolar.

### Data Analysis and Statistics

See Example 1.

### Experimental Procedures

Injections of cDNAs encoding for the human alpha3beta4, alpha3alpha5beta4, alpha4beta2, and alpha7 were performed in at least 95 oocytes were performed using a proprietary automated injection and receptor expression examined at least two days later. Oocytes were poked with two electrodes and their membrane potential maintained, unless indicated, at −80 mV throughout the experiment.

A protocol to determine agonistic activity in the oocytes expressing the nAChRs was developed. The cells were exposed to a series of increasing concentrations of a compound agonist for 10 seconds each at two minute intervals. The current evoked from exposure to the agonist at each concentration was recorded. For oocytes expressing alpha7 nAChRs, exposure to the agonist was reduced to 5 seconds. This process was repeated for each agonist tested against each type of nAChR expressing cell.

### Agonistic Properties of Compound 1 Hemi-Galactarate Dihydrate

To evaluate the agonistic properties of compound 1 hemi-galactarate dihydrate a protocol of brief exposures to a series of growing concentrations of compound 1 hemi-galactarate dihydrate was used.

Experiments conducted at the human nAChRs confirmed that compound 1 hemi-galactarate dihydrate behaves as an agonist at the considered receptors. Notably, however, compound 1 hemi-galactarate dihydrate evoked larger currents than the endogenous ligand acetylcholine (ACh) at the heteromeric alpha4beta2 receptors and displayed also at higher potency for this receptor subtype.

Compound 1 hemi-galactarate dihydrate acts almost as a full agonist at nAChR alpha3beta4, yielding an EC₅₀ of 42.09±5.36 micromolar.

Compound 1 hemi-galactarate dihydrate acts almost as a full agonist at nAChR alpha3alpha5beta4, yielding an EC50 of 32.15±2.16 μM.

Compound 1 hemi-galactarate dihydrate acts as a more potent agonist than ACh at nAChR alpha4beta2, yielding an EC50 of 48.82±17.41 μM.

Compound 1 hemi-galactarate dihydrate is a more potent agonist than ACh at nAChR alpha7. The EC₅₀ of 1261±500 μM is only an estimation given the small fraction of current evoked by compound 1 hemi-galactarate dihydrate.

### Agonistic Properties of Varenicline Tartrate

Varenicline tartrate acts as a very poor agonist at the human alpha4beta2 receptors and causes a major inhibition of the subsequent ACh-evoked current. In contrast, varenicline tartrate is a full agonist at the alpha7 receptors with an EC50 at about 15 μM. and causes no significant inhibition of the subsequent ACh-evoked current.

Data obtained for varenicline tartrate confirmed that this molecule acts as a partial agonist at the heteromeric alpha3beta4, alpha3alpha5beta4 and alpha4beta2 but was able to evoke responses equivalent to ACh at the alpha7 receptors.

### Antagonist Properties of Compound 1 Hemi-Galactarate Dihydrate and Varenicline Tartrate

Determination of the antagonistic activity of compound 1 hemi-galactarate dihydrate and varenicline tartrate was done using a cumulative exposure in the low concentration range. Results obtained for compound 1 hemi-galactarate dihydrate revealed that this compound inhibits in a concentration-dependent manner the alpha3alpha5beta4 and alpha4beta2 receptor and that inhibition was incomplete and reached a plateau for the highest concentrations tested. In contrast, no significant inhibition was observed either at alpha3beta4 or alpha7 receptors within the range tested in these experiments.

Antagonistic activity of varenicline tartrate was observed at alpha3alpha5beta4, alpha4beta2 and alpha7 receptors whereas the alpha3beta4 receptors displayed hardly any inhibition for concentrations up to 300 nM. Results obtained at the human alpha3beta4 illustrate that varenicline tartrate causes no significant inhibition of the ACh-evoked current up to concentration of about 300 nM. These data are in good agreement with previous report and that varenicline tartrate will act as an agonist only for concentrations in the micromolar range. Experiments conducted at oocytes injected with alpha3alpha5beta4 reveal, however, a different pattern as a small but significant inhibition of the ACh-evoked responses can be observed even for exposures in the nanomolar range. The maximal inhibition reached about 45% at 300 nM. Data obtained for varenicline tartrate at the human alpha4beta2 illustrate perfectly the inhibition caused by a sustained exposure to this molecule. Whereas a progressive inhibition of the ACh-evoked current is clearly observed as a function of the concentration, data reveals that the inhibition reaches a maximal at 78% which is in good agreement with the partial agonist activity of varenicline tartrate. These results illustrate that varenicline tartrate also causes a desensitization of the alpha7 nAChRs.

### Sustained Exposure to 300 μM Varenicline Tartrate and its Recovery

As it is expected that receptors can be exposed for a sustained duration to a rather high concentration of varenicline tartrate additional experiments were conducted at the human alpha3beta4 and alpha4beta2. These data illustrate the differences in sensitivity of these two nAChR subtypes and that while exposure to 300 μM varenicline tartrate evokes a rather large response at the human alpha3beta4 it causes only a small activation at the alpha4beta2. Moreover, the desensitization caused by varenicline tartrate shows a long-lasting effect that cannot be recovered even after 36 minutes wash.

Altogether, these data illustrate that expression of human nAChRs in Xenopus oocytes provides an efficient model for testing the functional agonistic and antagonistic activity for a given compound and that agonist cause desensitization of the receptors at lower concentrations than those required for activation.

### Example 3: Measurement of Maximum Plasma Concentration

This example describes a method of measuring the maximal plasma concentration (Cmax) of a nAChR agonist, or a pharmaceutically acceptable salt thereof, in an individual after administration of a pharmaceutical formulation comprising the nAChR agonist, or a pharmaceutically acceptable salt thereof.

### Dosing

A dose of a nAChR agonist, or a pharmaceutically acceptable salt thereof, is administered to an individual. Examples of dose and volume ranges are described herein.

### Blood Collection and Sample Processing

A blood sample is collected from an individual before administration of a nAChR agonist, or a pharmaceutically acceptable salt thereof. After administration of the nAChR agonist, or a pharmaceutically acceptable salt thereof, to the individual, multiple blood samples are collected over a period of time at certain intervals. For instance, blood samples are collected at 10 minutes, 20 minutes, 30 minutes, 1 hour, 2 hours, 3 hours, and 4 hours after administration of the nAChR agonist, or a pharmaceutically acceptable salt thereof.

Blood samples (4 mL in K2EDTA tubes) are collected from an arm vein into Vacutainer tubes containing K2EDTA. Blood samples are mixed gently and maintained chilled until centrifuged within 2 hours of collection.

Plasma samples are separated into two equivalent aliquots and labeled with the individual's identification and nominal time point and stored frozen (at <−70° C.) until analysis.

### Bioanalytical Methods for Pharmacokinetic Samples

Blood samples from the individual are processed to obtain plasma, and plasma concentrations of nAChR agonist, or a pharmaceutically acceptable salt thereof, using liquid chromatography followed by tandem mass spectrometry (LC-MS/MS) bioanalytical method (high performance liquid chromatography [HPLC] with mass spectrometric detection).

### Pharmacokinetic Analysis

All pharmacokinetic (PK) analyses and reporting are performed according to generally accepted standard operating procedures and protocol specifications. Pharmacokinetic parameters are calculated by use of generally accepted software tools, for instance Phoenix® WinNonlin® 8.0.

A plasma concentration time plot is created by plotting the concentration of the nAChR agonist, or pharmaceutically acceptable salt thereof, as a function of time. Cmax is calculated by inspection of an individual's plasma concentration time plot. Another Cmax related parameter is the maximum observed concentration of the nAChR agonist, or a pharmaceutically acceptable salt thereof, divided by milligram dose, (Cmax/D). The Cmax is calculated for an individual after a treatment. If the Cmax is calculated for more than one individual, then, the arithmetic mean Cmax and median Cmax may also be calculated for the group.

### Example 4: Measured Cmax after Treatment with Compound 1 Hemi-Galactarate Dihydrate

The blood plasma of 30 individuals treated with 1.1% compound 1 free base (2.0% compound 1 hemi-galactarate dihydrate) via a nasal spray (200 microliters total volume; about 2216 micrograms of compound 1 free base) was analyzed using the protocol described in Example 3. The mean Cmax of compound 1 measured was 2.92 ng/mL.

### Example 5: Compound 1 Citrate and Varenicline Tartrate Agonist Activity in Human nAChR Expressed in Xenopus Oocytes

This example describes experiments to characterize the agonist activity of compound 1 citrate at the human neuronal nicotinic acetylcholine receptors (nAChRs) alpha3beta4, alpha3alpha5beta4, alpha4beta2, alpha4alpha6beta2, and alpha7 expressed in Xenopus oocytes. In addition, the effect of varenicline tartrate on alpha4alpha6beta2 expressed in Xenopus oocytes was measured.

### Methods

Test compounds: compound 1 citrate, varenicline tartrate

Reference compound: acetylcholine chloride

Test System: See Example 1.

Oocytes Preparation: See Example 1.

Electrophysiological Recordings: See Example 1.

Agonist Preparation: See Example 1. The test concentrations of compound 1 citrate and varenicline tartrate were 0.1, 0.3, 1, 3, 10, 30, 100, 300 (micromolar). The test concentration of acetylcholine chloride was 1280 micromolar.

Data Analysis and Statistics: See Example 1.

Experimental Procedures: Injections of cDNAs encoding for the human alpha3beta4, alpha3alpha5beta4, alpha4beta2, alpha4alpha6beta2, and alpha7 were performed in at least 95 oocytes using a proprietary automated injection device. Cellular receptor expression was examined at least two days later. Oocytes were poked with two electrodes and their membrane potential maintained, unless indicated, at −80 mV throughout the experiment.

A protocol to determine agonistic activity in the oocytes expressing the nAChRs was developed. Oocytes expressing a nAChR subtype were exposed to a brief test pulse of acetylcholine (1280 micromolar) while the holding current and response was evaluated. Cells displaying robust currents were washed for 90 seconds. The cells were then exposed to a series of increasing concentrations of a nAChR agonist for 10 seconds each at two minute intervals. The nAChR agonist test concentrations were 0.1, 0.3, 1, 3, 10, 30, 100, 300 (micromolar). The current evoked from exposure to the agonist at each concentration was recorded. For oocytes expressing alpha7 nAChRs, exposure to the agonist was reduced to 5 seconds. This process was repeated for each agonist tested against each type of nAChR expressing cell.

### Results

The effects of compound 1 citrate at human neuronal nicotinic acetylcholine receptors expressed in Xenopus oocytes and investigated with two electrode voltage clamp. Determination of the agonistic effects of compound 1 citrate at the alpha3beta4, alpha3alpha5beta4, alpha4beta2, alpha4alpha6beta2, and alpha7 receptors revealed that this compound acts, in the 10 to 300 micromolar range, as an agonist at these receptors. Noticeable differences were, however, observed in the response between receptor subtypes. Namely, compound 1 citrate was a weak partial agonist at the human alpha7 receptors at a 300 micromolar concentration. A 300 micromolar compound 1 citrate solution evoked a response of only 25 percent of the ACh-evoked current. In contrast, data recorded for nAChR subtypes alpha3beta4, alpha3alpha5beta4, alpha4beta2 and alpha4alpha6beta2 revealed that compound 1 citrate acts as an almost full agonist at these receptors.

Compound 1 citrate acts almost as a full agonist at nAChR alpha3beta4, yielding an EC₅₀ of 34.87±4.53 micromolar.

Compound 1 citrate acts almost as a full agonist at nAChR alpha3alpha5beta4, yielding an EC₅₀ of 83.00±9.05 micromolar.

Compound 1 citrate acts almost as a full agonist at nAChR alpha4beta2, yielding an EC₅₀ of 13.48±2.06 micromolar. Significant inhibition of the current evoked by the second Ach exposure is attributed to the desensitization caused by compound 1 citrate exposure.

Compound 1 citrate acts almost as a full agonist at nAChR alpha4alpha6beta2, yielding an EC₅₀ of 13.14±3.68 micromolar.

Compound 1 citrate acts as a poor agonist at nAChR alpha7 evoking at 300 micromolar only 25 percent of the maximal ACh-evoked current, yielding an EC₅₀ of 125.63±28.52 micromolar. The EC₅₀ is an only an estimation given the small fraction of current evoked by compound 1 citrate.

Varenicline acts as weak partial agonist at nAChR alpha4alpha6beta2. At concentrations from 0.1 micromolar to 300 micromolar, varenicline evoked a 6% or less response compared to the ACh response. The calculated EC₅₀ was 5.02±1.21 micromolar. Exposure to varenicline was observed to cause a significant inhibition of the subsequent ACh-evoked current.

### Example 6: 7-Day Tolerability and Pharmacokinetic Study of PAM and nAChR Agonist Combination Administration in Animals

Intranasal administration of a nAChR agonist in combination with administration of a PAM can be performed in rodents (rats and mice), rabbits, and larger species, including dogs and non-human primates, though experimental techniques (e.g., dose volume per nostril, delivery device) for administration of test article may differ across species. The use of rats and dogs or monkey are most common for evaluation of intranasal drug products in rodents and non-rodents, respectively. However, rabbits are also used, and methods for the histopathological evaluation of the rabbit nasal cavity in safety assessment studies have been developed by Pereira et al. (Pereira M E, Macri N P, Creasy D M. Evaluation of the rabbit nasal cavity in inhalation studies and a comparison with other common laboratory species and man. Toxicologic Pathology. 2011. 39:893-900.) The PAM may also be administered intranasally. Alternatively, the PAM may be administered by other routes, such as orally or topically.

Rats

The potential toxicity and toxicokinetic behavior of PAM and nAChR Agonist Combination may be assessed in a 7-day intranasal administration study in rats (e.g., Sprague Dawley rats). 60 μL of a nAChR agonist (e.g., Compound 1) at dose concentrations of 0%, 0.5%, 2%, and 6% (e.g. Compound 1, based on the hemi-galactarate salt) is intranasally administered to rats 3 times per day for 7 days. The 60 μL total volume administered is split equally between each nostril (30 μL per nostril per dose), and is administered 3 times daily (a total daily volume of 180 μL).

In addition, the rats are treated with a PAM (e.g., Br-PBTC or NS-9283). A range of PAM concentrations may be tested. The dose frequency and timing of the PAM administration relative to the nAChR agonist may vary. For instance, the PAM may be administered daily, a few times, or once throughout the 7-day study.

The following parameters and end points may be evaluated: clinical signs, body weights, body weight gain, food consumption, ophthalmology, toxicokinetic parameters, gross necropsy findings, organ weights, and histopathologic examination of select tissues, including nasal cavity at levels I-IV and detailed ocular histopathology for all dose groups; histopathology for brain, pharynx, larynx, trachea, lung, and select target tissues (i.e., adrenal gland, liver, and spleen); and gross lesions. The clinical observations may provide toxicologically relevant information. The results of the treated rats are compared to a control group administered phosphate buffered saline.

With respect to toxicokinetic measurements, the following parameters are calculated for the nAChR agonist and the PAM:

| Parameter | Description of Parameter |
|---|---|
| Tmax | The time after dosing at which the maximum observed concentration was |
| | observed. |
| Cmax | The maximum observed concentration measured after dosing. |
| Cmax/D | The maximum observed concentration divided by the total daily dose |
| | administered. |
| C1 | The peak concentration after the 1^{st} instillation. |
| C2 | The peak concentration after the 2^{nd} instillation. |
| C3 | The peak concentration after the 3^{rd} instillation. |
| AUC(0-t) | The area under the concentration versus time curve from the start of dose |
| | administration to the last observed quantifiable concentration using the |
| | linear or linear/log trapezoidal method. |
| AUC(0-t)/D | The AUC(0-t) divided by the dose administered. |
| RAUC | The area under the curve from T1 to T2 on Day 7 at steady state divided by |
| | the area under the curve from T1 to T2 during the initial dosing interval on |
| | Day 1. |

The parameters are generated from composite concentrations in blood plasma over time from Days 1 and 7 whenever practical. Based on these parameters, the absorption rate, half-life, and insight into accumulation and clearance of the nAChR agonist or PAM may be calculated.

Rabbits

Alternatively, rabbits (e.g., New Zealand rabbits) may be selected for evaluation of nAChR agonist and PAM in intranasal toxicity and toxicokinetic studies.

Dose Administration. The first day of dose administration is defined as Day 1. The nAChR agonist test article and control article are administered by use of an intranasal mucosal atomizer attached to a syringe. Approximately 100 μL of the control article, phosphate-buffered saline solution, is sprayed into each nostril of animals in Group 1. Approximately 100 μL of the test article Compound 1, at an assigned concentration, is sprayed into each nostril of animals in Groups 2, 3, and 4. The animals in Groups 1 and 4 are dosed two times a day, approximately 8 hours apart. The animals in Groups 2 and 3 are dosed once daily. All animals are dosed for seven (7) consecutive days.

In addition, a PAM (e.g. Br-PBTC) is administered to the test rabbits in Groups 2, 3, and 4. A range of PAM concentrations may be tested. For instance, the concentration of the PAM may range from 10× lower than the concentration of the nAChR agonist to a similar concentration of the nAChR agonist. The PAM may be administered separately or simultaneously with the nAChR agonist. Alternatively, the dose frequency and timing of the PAM administration may vary. For instance, the PAM may be administered daily, a few times, or once throughout the 7-day study. The PAM may be administered with each administration of the nAChR agonist. With respect to the route of administration, the PAM may be administered intranasally. Alternatively, the PAM may be administered orally.

The study protocol is summarized below.

| Objective | To evaluate the potential toxicity and toxicokinetics of Compound 1 | | |
|---|---|---|---|
| | (intranasal administration) and Br-PBTC. | | |
| Test Article | Compound 1 and Br-PBTC | | |
| Test System | New Zealand White rabbits, 24 total (male) | | |
| Experimental | 7-day dosing | | |
| Design | | | |
| | | Dosing | |
| | | Frequency of | Number of |
| | Group | Compound 1 | Animals |
| | Vehicle (PBS) | 2x Daily | 6 |
| | Compound 1 1% | 1x Daily | 6 |
| | and Br-PBTC | | |
| | Compound 1 2.5% | 1x Daily | 6 |
| | and Br-PBTC | | |
| | Compound 1 2.5% | 2x Daily | 6 |
| | and Br-PBTC | | |

| | |
|---|---|
| Route | Compound 1: Intranasal instillation via micropipette, 100 μL/nostril. |
| | Br-PBTC: intranasal or oral administration |
| Safety | Clinical Observations |
| Endpoints | Intranasal Assessment: Intranasal irritation will be performed using a |
| | Draize scoring scale |
| | Body Weights |
| Toxicokinetics | Blood samples will be collected from all animals after the first dose (1 hr |
| | post-dose) and at termination (24 h post-dose). |
| Necropsy | Complete with full tissue collection and organ weights (liver, lungs, heart, |
| | kidneys, brain, adrenal glands, testes, thyroid, pituitary, spleen, and thymus). |
| Histopathology | Histopathological assessment of the nasal cavity (right and left nostrils) and |
| | respiratory tissues (pharynx, larynx, mediastinal lymph nodes, and trachea |
| | with lung lobes) will be performed on 3 animals per group. Right nasal |
| | meatus mucosa and front lobe of the brain will be harvested for tissue PK |
| | from the remaining 3 animals per group. |

Histopathology. For histopathology evaluation of the tissues, each lesion is listed and coded by the most specific topographic and morphologic diagnoses, severity, and distribution. A five-step/severity grading system (minimal, mild, moderate, marked, and severe) is used to define gradable lesions. Records of gross findings for a specimen from postmortem observations are available to the pathologist when examining that specimen microscopically.

Clinical Observations. Animals are observed once after each dosing, 30-60 minutes post dose for clinical signs of toxicity. Findings are recorded as they are observed. Clinical observations include, but are not limited to, changes in the skin, fur, eyes and mucous membranes, respiratory system, circulatory system, autonomic central nervous system, somatomotor activity, locomotor activity, and behavioral pattern.

Intranasal/Nasal Assessment. Intranasal/nasal assessments are performed before termination by using a nasal speculum. Intranasal irritation is performed using a Draize scoring scale. The assessment includes observation and recording of sneezing. The Draize classification system for scoring skin irritation is summarized below.

| | Value |
|---|---|
| Erythema and Eschar formation | |
| No erythema | 0 |
| Very slight erythema (barely perceptible) | 1 |
| Well-defined erythema | 2 |
| Moderate erythema | 3 |
| Severe erythema (beet redness) to slight eschar formation | 4 |
| (preventing grading of erythema) | |
| Edema Formation Value | |
| No edema | 0 |
| Very slight edema (barely perceptible) | 1 |
| Well-defined edema (edges of area well-defined by definite | 2 |
| raising) | |
| Moderate edema (raised approximately 1 mm) | 3 |
| Severe edema (raised more than 1 mm and extending beyond | 4 |
| area of exposure) | |

### Example 7: Pharmacokinetic Measurements in Human Subjects after 28 Days of Intranasal Administration of Compound 1 Hemigalactarate Salt

This example describes a study to determine the plasma systemic exposure and pharmacokinetics after four weeks of treatment with 1.1% Compound 1 (2.0% Hemigalactarate Salt), and after a single bilateral nasal spray on the 29^{th} day. The results demonstrate that Compound 1 is detected in all subjects 10 minutes after administration, indicating rapid nasal absorption of Compound 1. In addition, this example demonstrates that Compound 1 exhibits low systemic exposure after intranasal administration and should not exhibit any accumulation in systemic circulation after repeated administration.

Seven patients were administered 1.1% Compound 1 (2.0% hemigalactarate salt) delivered as a 50 μL intranasal spray in each nostril, BID for 28 days. On Day 29, a single intranasal administration of 1.1% Compound 1 (2.0% hemigalactarate salt) was provided to the patients.

Blood plasma for the patients was collected on Day 29 predose (time 0) and after a series of time points (10 min, 20 min, 30 min, 1 hr, 2 hrs, 4 hrs, 5 hrs, 6 hrs, 7 hrs, 8 hrs, and 24 hrs) after a single administration of drug. The blood plasma was analyzed for Compound 1.

The following pharmacokinetic parameters for Compound 1 were calculated:
Cmax: Maximum observed concentration, observed by inspection of individual study participant plasma concentration time plots.
Tmax: Time of maximum observed concentration, obtained directly from the observed concentration time data.
AUClast: The area under the plasma concentration time curve, from time 0 to the last measurable non-zero concentration, calculated by a combination of linear and logarithmic trapezoidal methods (Linear up/log down method).
AUC0-4h: The area under the plasma concentration time curve, from time 0 to the 4 h time point, calculated by a combination of linear and logarithmic trapezoidal methods (Linear up/log down method).
AUCinf: The area from zero time (pre-dose) extrapolated to infinite time (AUCinf) will be calculated as follows: AUCinf=AUClast+Cz/λz.
AUC % Extrap: The percentage of AUCinf obtained by extrapolation (AUC % Extrap) will be calculated as follows:

$$AUC \% Extrap = \frac{( AUCinf - AUClast )}{AUCinf} \times 100$$

Half-life: Terminal elimination half-life.

Plasma Concentration Data. After 28 days of BID administration of 1.1% Compound 1, Compound 1 was detected at time zero (predose) in 4 out of 7 subjects with a mean concentration of 0.264 ng/mL. Ten minutes after administration, Compound 1 was detected in plasma from all subjects, indicating rapid nasal absorption. The mean Compound 1 plasma exposure increased with time after intranasal administration with peak of exposure of 2.07 ng/mL at 1 h and then declined through the 24 h sampling period. The decline was biphasic with a plateau between 2 and 4 h. Four of the seven subjects had quantifiable levels at 5 h post dose and there was a loss of one subject with quantifiable Compound 1 at each of the next three time points, 6, 7 and 8 h. There was only one subject with quantifiable Compound 1 8 h post administration and no subjects had quantifiable Compound 1 above the lower limit of quantification of 0.200 ng/mL at 24 h post administration.

Pharmacokinetic Parameter Data. Compound 1 mean (SD) Cmax was 2.26±1.96 ng/mL with an individual subject maximum of 5.49 ng/mL from the seven subjects. The median Tmax occurred at 0.50 h and the mean (SD) half-life was 1.58±0.818 h. The AUC % Extrap in four of the seven subjects was >20%, which should exclude the calculation of half-life and AUCinf, however, these individual subject values were reported and included in the summary statistics for the PK parameters AUCinf and Half-life. Mean (SD) AUClast was variable at 5.22±4.52 h*ng/mL and the maximum single individual value at 12.1 h*ng/mL. However, one subject with the lowest AUC % Extrap at 3.69% was also the subject with the largest AUClast and AUCinf at 12.1 h*ng/mL and 12.5 h*ng/mL, respectively. The mean (SD) AUCinf was 21.3% greater than mean AUClast at 6.22±4.63 h*ng/mL. The maximum individual subject half-life was 2.83 h, which would suggest that in general if Compound 1 was administered every 12 hours, then >94% (4 half-lives) of compound 1 would clear from systemic circulation for all subjects before the next administration. Overall, systemic exposure for Compound 1 was low.

The pharmacokinetic results are summarized in the table below.

| | | | | | | AUC % |
|---|---|---|---|---|---|---|
| | Half-life | Tmax | Cmax | AUClast | AUCinf | Extrap |
| | (h) | (h) | (ng/mL) | (h*ng/mL) | (h*ng/mL) | (%) |
| N | 7 | 7 | 7 | 7 | 7 | 7 |
| Mean | 1.58 | 0.70 | 2.26 | 5.22 | 6.22 | 21.3 |
| SD | 0.818 | 0.38 | 1.96 | 4.52 | 4.63 | 14.6 |
| Min | 0.852 | 0.17 | 0.621 | 0.801 | 1.21 | 3.67 |
| Median | 1.12 | 0.50 | 1.35 | 4.35 | 5.67 | 23.3 |
| Max | 2.83 | 1.2 | 5.49 | 12.1 | 12.5 | 41.1 |

Conclusion. After 28 days of BID administration of 1.1% Compound 1, Compound 1 exhibits low systemic exposure with 4 of 7 subjects (57.1%), with a mean concentration of 0.264 ng/mL, exhibiting predose systemic exposure. Ten minutes after administration, Compound 1 was detected in plasma from all subjects, indicating rapid nasal absorption. The clearance of Compound 1 was biphasic in nature with a plateau between 2 and 4 h, which may suggest a possible oral component of absorption to the systemic profile. However, there was only one subject with quantifiable amounts of Compound 1 at 8 h post administration and no subjects 24 h post administration. This correlated with the observed mean (SD) half-life of 1.58±0.818 h. The maximum individual subject half-life was 2.83 h, which would suggest that in general if Compound 1 was administered every 12 hours, >94% (4 half-lives) of Compound 1 would clear from systemic circulation for all subjects before the next administration. Overall, systemic exposure for Compound 1 was low with a mean (SD) Cmax of 2.26±1.96 ng/mL, with an individual subject maximum of 5.49 ng/mL from the seven subjects.

1-91. (canceled)

92. A method of treating keratitis, dry eye disease, increasing tear production, or reducing ocular discomfort in an individual in need thereof, comprising administering
a first dose, and optionally one or more subsequent doses, of an effective amount of a nicotinic acetylcholine receptor (nAChR) agonist, or a pharmaceutically acceptable salt thereof, and
a first dose, and optionally one or more subsequent doses, of an effective amount of a positive allosteric modulator (PAM), or a pharmaceutically acceptable salt thereof,
into a nasal cavity of the individual in need thereof;
wherein the nAChR agonist, or a pharmaceutically acceptable salt thereof, is varenicline, or a pharmaceutically acceptable salt thereof, or compound 1 having the structure 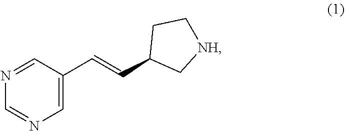 [SMILES: C(=C\[C@H]1CCNC1)/c1cncnc1]
or a pharmaceutically acceptable salt thereof.

93. The method of claim 92, wherein keratitis is neurotrophic keratitis.

94. A method of treating keratitis, dry eye disease, increasing tear production, or reducing ocular discomfort in an individual in need thereof, comprising administering
an effective amount of a positive allosteric modulator (PAM), or a pharmaceutically acceptable salt thereof, into a nasal cavity of the individual in need thereof.

95. The method of claim 92, wherein 5-4000 micrograms of the nAChR agonist, or a corresponding amount of a pharmaceutically acceptable salt thereof, per dose is administered to the individual.

96. The method of claim 92, wherein the nAChR agonist, or a pharmaceutically acceptable salt thereof, is administered in a pharmaceutical formulation for nasal administration comprising between 1 mg/mL and 40 mg/mL of nAChR agonist, or a corresponding amount of a pharmaceutically acceptable salt thereof.

97. The method of claim 92, wherein the nAChR agonist, or a pharmaceutically acceptable salt thereof, is administered in a pharmaceutical formulation for nasal administration, and the total volume of the pharmaceutical formulation administered per dose of the nAChR agonist, or a pharmaceutically acceptable salt thereof, to the individual is 50 microliters-250 microliters.

98. The method of claim 92, wherein the individual has undergone Lasik surgery within 2 weeks or is scheduled to undergo Lasik surgery within 2 weeks.

99. The method of claim 92, wherein the nAChR agonist is varenicline, or a pharmaceutically acceptable salt thereof.

100. The method of claim 92, wherein the nAChR agonist is compound 1, or a pharmaceutically acceptable salt thereof.

101. The method of claim 92, wherein the PAM is selected from the group consisting of 17-beta-Estradiol, (R)-7-bromo-N-(piperidin-3-yl)benzo[b]thiophene-2-carboxamide, ivermectin, galantamine, genistein, 5-hydroxyindole, 4BP-TQS, A-86774, CCMI, levamisole, morantel, LY-2087101, mecamylamine, menthol, NS206, NS1738, NS9283, PNU-120596, RO5126946, TBS-345, dFBR, and HEPES, or a pharmaceutically acceptable salt of any of the foregoing.

102. The method of claim 92, wherein the PAM is (R)-7-bromo-N-(piperidin-3-yl)benzo[b]thiophene-2-carboxamide, or a pharmaceutically acceptable salt thereof.

103. The method of claim 92, wherein the individual in need thereof has a blood plasma Cmax of the nAChR agonist, or a pharmaceutically acceptable salt thereof, of less than 5 ng/mL.

104. The method of claim 92, wherein the effective treatment of the individual is indicated by one or more of the tests selected from the group consisting of
a) Eye Dryness score test on a visual analog scale,
b) Schirmer's test,
c) Corneal Fluorescein Staining test, and
d) Ocular Surface Disease Index test.

105. A pharmaceutical formulation for local administration into the nasal cavity of an individual comprising a nAChR agonist, or a pharmaceutically acceptable salt thereof, and a PAM, or a pharmaceutically acceptable salt thereof,
wherein the nAChR agonist, or a pharmaceutically acceptable salt thereof, is varenicline, or a pharmaceutically acceptable salt thereof, or compound 1 having the structure 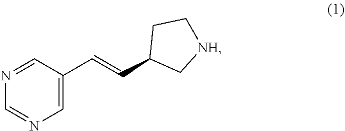 [SMILES: C(=C\[C@H]1CCNC1)/c1cncnc1]
or a pharmaceutically acceptable salt thereof, formulated for nasal administration.

106. The pharmaceutical formulation of claim 105, comprising 5-4000 micrograms of the nAChR agonist, or a corresponding amount of a pharmaceutically acceptable salt thereof, per dose.

107. The pharmaceutical formulation of claim 105, wherein the pharmaceutical formulation comprises between 1 mg/mL and 40 mg/mL of nAChR agonist, or a corresponding amount of a pharmaceutically acceptable salt thereof.

108. The pharmaceutical formulation of claim 105, wherein the nAChR agonist is varenicline, or a pharmaceutically acceptable salt thereof.

109. The pharmaceutical formulation of claim 105, wherein the nAChR agonist is compound 1, or a pharmaceutically acceptable salt thereof.

110. The pharmaceutical formulation of claim 105, wherein the PAM is selected from the group consisting of 17-beta-Estradiol, (R)-7-bromo-N-(piperidin-3-yl)benzo[b]thiophene-2-carboxamide, ivermectin, galantamine, genistein, 5-hydroxyindole, 4BP-TQS, A-86774, CCMI, levamisole, morantel, LY-2087101, mecamylamine, menthol, NS206, NS1738, NS9283, PNU-120596, RO5126946, TBS-345, dFBR, and HEPES, or a pharmaceutically acceptable salt of any of the foregoing.

111. The pharmaceutical formulation of claim 105, wherein the PAM is (R)-7-bromo-N-(piperidin-3-yl)benzo[b]thiophene-2-carboxamide or NS9283, or a pharmaceutically acceptable salt of either of the foregoing.

112. The pharmaceutical formulation of claim 105, wherein the pharmaceutical formulation is a liquid, suspension, aerosol, gel, ointment, dry powder, cream, paste, balm, or nasal spray.

113. The pharmaceutical formulation of claim 105, wherein the pharmaceutical formulation is administered into the nasal cavity by a syringe, dropper, bottle nebulizer, atomization pump, inhaler, powder spray device, vaporizer, patch, medicated stick, pipette, or jet of liquid.

114. The pharmaceutical formulation of claim 105, for use in treating keratitis, dry eye disease, increasing tear production, or improving ocular discomfort in an individual in need thereof.

115. The pharmaceutical formulation of claim 114, wherein keratitis is neurotrophic keratitis.

116. A kit comprising a nAChR agonist, or a pharmaceutically acceptable salt thereof, and a PAM, or a pharmaceutically acceptable salt thereof,
for use in treating keratitis, dry eye disease, increasing tear production, or improving ocular discomfort in an individual in need thereof;
wherein the nAChR agonist, or the pharmaceutically acceptable salt thereof, is varenicline, or a pharmaceutically acceptable salt thereof, or compound 1 having the structure 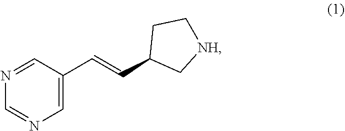 [SMILES: C(=C\[C@H]1CCNC1)/c1cncnc1]
or a pharmaceutically acceptable salt thereof.
